(19) United States
(12) Patent Application Publication
(10) Pub. No.: US 2018/0282403 A1
(43) Pub. Date: Oct. 4, 2018

(54) AGENTS FOR REDUCING THE ACTIVITY OF GDF15

(71) Applicant: GENAGON THERAPEUTICS AB, Solna (SE)

(72) Inventors: Olof Andries BLOKZIJL, Stockholm (SE); Johan Erik Simon FREDRIKSSON, Bromma (SE)

(21) Appl. No.: 15/746,050

(22) PCT Filed: Jul. 20, 2016
(86) PCT No.: PCT/EP2016/067338

§ 371 Date: Jan. 19, 2018

(30) Foreign Application Priority Data
Jul. 20, 2015 (GB) 1512733.5

(51) Int. Cl. C07K 16/22; C07K 14/705; C07K 16/28; A61P 35/04

(52) U.S. Cl. CPC C07K 16/22; C07K 14/705; C07K 16/2863; C07K 2319/30; C07K 2317/34; C07K 2317/76; A61P 35/04

(57) ABSTRACT

The present invention relates to agents for reducing the activity of GDF15 and in particular the use of such agents to treat or prevent conditions associated with elevated or unwanted levels of GDF15. The invention is based on the discovery that GDF15 binds to the receptors CLPTM1 and QRFPR and provides agents for such use in the form of binding agents capable of binding to the receptors and inhibiting the interaction between GDF15 and the receptor. Further agents include polypeptides derived from the receptors which are capable of binding to GDF15 and inhibiting its interaction with the receptors. Also provided are diagnostic methods based on detecting the interaction or an effect thereof, and cytotoxic immune cells modified to have a reduced level and/or activity of CLPTM1.

The present invention relates to agents for reducing the activity of GDF15, including therapeutic agents which may be used in a subject thereby to combat (i.e. to treat or prevent) conditions associated with increased, excessive or unwanted GDF15 levels and/or activity in the body. The present invention is based on the discovery of two different receptors for GDF15 in the body and accordingly, based on this identification of novel GDF15-receptors, the present invention more specifically provides fragments of the receptors for GDF15, and antibodies, or other affinity binding reagents, which bind the GDF15 receptors, for use in preventing GDF15 from binding to its receptors and/or otherwise inhibiting the effect of GDF15 (i.e. the effect of the binding of GDF15) at one or both of the receptors.

Growth and differentiation factor 15 (GDF15), also known as macrophage inhibitory cytokine 1 (MIC1), is a member of the TGF-β superfamily, and has a relatively low (24%) sequence homology with other members of the TGF-β superfamily. Elevated levels of GDF15 have been implicated in cancer, anorexia nervosa, osteoporosis, kidney disorders, pulmonary arterial hypertension, and cardiovascular disease, and also in cachexia and more generally in loss or suppression of appetite. GDF15 is a marker for mortality by any cause.

GDF15 has also been found to be overexpressed in Sickle Cell Disease, iron overload disorders such as Hereditary Spherocytosis, Congenital Dyserythropoietic Anaemia type I (Tamary et al. 2008. Blood 112, 5241-5244) and type II, Thalassemia (Tanno et al. 2007. Nat Med 13, 1096-1101), Refractory anaemia with ring-sideroblasts (RARS) (Ramirez et al. 2009. Br J Haematol 144, 251-262), and Pyruvate kinase deficiency (Finkenstedt et al. 2009. Br J Haematol. 144, 789-793).

Whilst GDF15 was first cloned in 1997, the receptor(s) of GDF15 have so far remained elusive.

GDF15 has a dual role in tumour pathology: the ligand induces apoptosis in early tumours, yet it is secreted in large amounts in late stage cancers, including prostate, breast, gastric (Kim et al., 2008. Carcinogenesis 29, 704-712), colorectal cancers, and glioblastomas (Albertoni et al., 2002. Oncogene 21, 4212-4219). In late stage disease, GDF15 contributes to tumour metastasis and is the most important biomarker for bone engagement in metastatic prostate cancer (Selander et al., 2007. Cancer Epidemiol Biomarkers Prev 16, 532-537; Wakchoure et al., 2009. Prostate 69, 652-661).

Evidence in the literature describes upregulation of GDF15 expression depending on the site of metastasis, with markedly elevated levels in subpopulations of prostate cancer cells infiltrating bone. GDF15 is also known to affect both osteoclasts and osteoblasts (Wakchoure et al. 2009. Prostate 69, 652-661).

GDF 15 also has a role in cachexia, the wasting of the body due to chronic illness, and in particular has been implicated in loss of appetite and cachexia in late stage cancers. Appetite stimulants are commonly used to compensate appetite loss in patients with late-stage cancer, but as yet there is currently no agent clinically available to target the molecular mechanism of GDF15. Thus, tumour-induced cachexia is difficult to reverse. Recent estimates show that up to 25% of fatal outcomes in cancer may be due to cachexia.

GDF15 is thought to exert its effects in cachexia through central nervous system mechanisms by acting on hypothalamic circuits. Systemic administration of GDF15 has been found to cause a decrease in the expression of NPY, a key appetite-stimulatory peptide, and an increase in the expression of POMC, a precursor of the suppressor of appetite α-MSH, in the arcuate nuclei of the hypothalamus. The arcuate nuclei regulate feeding behaviour and are located adjacently to the area postrema, where the blood-brain barrier (BBB) is permissive to systemic factors. Accordingly, elevated levels of GDF15 have been shown to be associated with suppression of appetite. Additionally, the degree of weight loss in patients with advanced prostate cancer correlates with serum levels of GDF15 over a 6-month period but not with the serum levels of other factors implicated in cachexia, such as TNF-a, IL-6 or IL-8.

Breit in US 2009/000481 proposes the use of GDF15 (MIC-1) modulating agents which increase or decrease the amount or activity of GDF15 in the body as a means of modulating appetite or body weight, including the use of anti-GDF15 antibodies or fragments thereof for treating decreased appetite and/or weight loss associated with cancer or other conditions in which GDF15 is over-expressed. Whilst these, and other, physiological and pathological roles of GDF15 have so far been elucidated, the identity of the receptor (or receptors) for this physiologically important protein have remained elusive. The inventors have identified for the first time two separate and structurally unrelated receptors for GDF15 using a tandem affinity purification (TAP) assay, and based on these findings, propose novel therapeutic agents for reducing the effects GFD15.

Two GDF15 receptors have been identified in the present application: the pyroglutamylated RFamide Peptide Receptor (QRFPR) and Cleft Lip and Palate Transmembrane protein 1 (CLPTM1).

QRFPR, also known as orexigenic neuropeptide QRFP receptor or G-protein coupled receptor 103 (GPR103), is a 7-transmembrane G-protein coupled protein receptor that comprises an N-terminal extracellular domain and three further extracellular domains (or loops). The amino acid sequence of human QRFPR is shown in SEQ ID NO.1. An in silico prediction of the topology of QRFPR suggests that the extracellular N-terminal domain of QRFPR (ECD1) comprises residues 1-46 (SEQ ID NO. 3); extracellular domain 2 (ECD2; loop 1) comprises residues 103-120 (SEQ ID NO. 4); extracellular domain 3 (ECD3; loop 2) comprises residues 184-212 (SEQ ID NO.5); and extracellular domain 4 (ECD 4; loop 3) comprises residues 293-311 (SEQ ID NO. 12). QRFPR is an orexigenic, or appetite stimulating, receptor that is highly expressed in the arcuate nuclei of the hypothalamus (Bruzzone et al., 2007. J Comp Neurol 503, 573-591; Ukena et al. 2013. Gen Comp Endocrinol 190, 42-46). Binding of the ligand RFamide 43 to QRFPR induces expression of the appetite-stimulatory peptide NPY and inhibits POMC expression (an appetite suppressant), stimulating feeding behaviour and regulating blood pressure (Takayasu et al., 2006. Proc Natl Acad Sci USA 103, 7438-7443). RFamine 43 and GDF15 thus have opposite roles in the expression of NPY and POMC and thus in the regulation of appetite. The findings, reported in more detail below, underlying the present invention that GDF15 may act as an antagonist at this receptor, antagonising the effects of endogenous agonistic ligands, are thus consistent with the known association of increased GDF15 levels with decreased appetite/cachexia.

We propose that the interaction of GDF15 and QRFPR interferes with the appetite-stimulating function of QRFPR with implications on negative regulation of feeding behavior. This leads then to the proposal underlying the present invention, that agents which interfere with the interaction of GDF15 and QRFPR may be used therapeutically to treat or prevent conditions associated with appetite suppression and/or weight loss, such as cachexia.

In addition to regulating appetite, the QRFPR receptor regulates bone formation (Baribault et al., 2006. Mol Cell Biol 26, 709-717). QRFPR knockout mice are slim and display a loss in bone mass. Also, the number of osteoclasts is reduced. QRFPR is known to be expressed in cells of the bone, including particularly osteoblasts and we have shown that QRFPR is up-regulated in both a number of different primary bone cancers, and in secondary bone micrometastases. Accordingly, we now further propose that the interaction between GDF15 and QRFPR may be important in disorders of, or involving, bone, including both primary bone cancers, and metastasis of other cancers (e.g. prostate cancer) to bone and that agents which interfere in this interaction represent a new treatment modality to treat or prevent such conditions.

In work leading up to the present invention we have shown that GDF15 is able to bind to QRFPR and cause a decrease in its expression level at the cell surface by dual mechanisms. GDF15 induces receptor internalization and degradation by the proteasome, and also induces the shedding of the receptor from the cell surface by secretion of QRFPR positive exosomes. Whilst not wishing to be bound by theory, it is thought that the interaction between GDF15 and QRFPR interferes with the activity of QRFPR through down-regulating the level of QRFPR available at the cell surface. Blocking the interaction between GDF15 and QRFPR is thus proposed as a possible treatment for pathologies associated with elevated levels of GDF15, such as those discussed above.

The second receptor identified for GDF15 is CLPTM1 (The amino acid sequence of this receptor in humans is shown in SEQ ID NO.2). CLPTM1 is a transmembrane protein with a ˜350 amino acid extracellular domain (the amino acid sequence of the ECD of human CLPTM1 is shown in SEQ ID NO.14 and has 353 amino acids (representing amino acids 2-354 of SEQ ID NO:1)). CLPTM1 has an unusual expression pattern and is expressed, for instance, in the cells of the immune system. In particular, CLPTM1 is known to be expressed in Natural Killer cells (NK-cells) and macrophages, and we have further shown that it may be expressed by other cells of the immune system, such as various classes of lymphocytes, particularly various classes of T-lymphocytes; particular sub-sets of CD4+, CD8+ T-cells may express CLPTM1, as may particular subsets of CD3-CD45+ non-T-cells. Thus, CLPTM1 may be expressed by various immune cells.

Unlike QRFPR, GDF15 binding to CLPTM1 does not result in the loss of the receptor from the cell surface. Rather, stimulation of NK-92 cells by GDF15 has been found in the work underlying the present invention to induce the co-localisation of TGFbRI (ALK5) and TGFbRII with CLPTM1, and results in the phosphorylation of GSK3b. GDF15 had previously been associated with GSK3 phosphorylation, and phosphorylated (9/21) GSK3B is associated with reduced activation and cytotoxicity of cells of the innate immune system. The observation that CLPTM1 associates with the TGBb receptor complex upon stimulation with GDF15 thus explains how GDF15 might decrease NK cell cytotoxicity. This supports the proposal that by inhibiting the interaction of GDF15 with its receptors, including particularly CLPTM1, which may be expressed on various immune cells, the immunosuppressive effects of GDF15 may be reduced.

In normal physiology, high levels of GDF15 are secreted by placental cells. We believe that GDF15 may play a physiological role in immune regulation in the placenta, down-regulating the immune response to placental cells and protecting the placenta from immune attack. Both macrophages and Natural Killer cells (NK-cells) pose a severe threat to the “foreign” cells of the placenta and must be locally suppressed. GDF15 may be secreted in an unprocessed form comprising a pro-peptide pro-GDF15, that is thought to associate with components of the extracellular matrix of the cells that secrete it, creating a pool of latent GDF15 in the vicinity of the cell. We propose that high local concentrations of GDF15 at the surface of the cells of the placenta have the potential to reduce the activity of both macrophages and NK-cells and potentially other GDF15 responsive immune cells. Importantly, we further believe that GDF15 may play a similar role in immune evasion by cancers. A large number of tumours secrete GDF15, effectively increasing the local concentration of GDF15 in the tumour stroma and at sites of metastasis, in a manner similar to in the placenta. By targeting QRFPR and/or CLPTM1 tumours may reduce the activity of NK, macrophages and/or other immune cells and thereby protect themselves from the cellular immune response. We now accordingly propose a further utility for agents which inhibit the interaction between GDF15 and the receptors QRFPR and/or CLPTM1 in inhibiting immune evasion of, or immune tolerance induced by, cancers, particularly in inhibiting metastasis, as well as a more general utility in inhibiting GDF15-induced immune suppression.

In particular, we show in the Examples below that peptides from both the third and fourth extracellular domains (ECDs) of QRFPR (loop 2 and loop 3 of QRFPR) are capable of binding to GDF15. We thus propose that the GDF15 binding site in QRFPR lies in both ECD3 (SEQ ID NO.5) and ECD4 (SEQ ID NO. 12), which is believed to be distinct from the binding site for the endogenous agonistic ligands. These protein loops each comprise ˜20 amino acids, and GDF15 is thought to bind to both of these loops simultaneously when binding to this receptor. The studies reported in the Examples below further demonstrate that peptides comprising sequences with a high degree of sequence identity to the amino acid sequences of these loops are effective at preventing GDF15-induced loss of QRFPR from the cell surface whilst also sparing the ability of QRFPR to be activated by its native ligands, indicating that these peptides are capable of specifically blocking the GDF15-QRFPR interaction by binding to GDF15. Peptides derived from extracellular domains 3 and 4 of QRFPR (loops 2 and 3) thus represent promising potential therapeutic agents for blocking the interaction between GDF15 and its receptors. Furthermore, antibodies binding the extracellular domains of QRFPR are also shown in the Examples below to be able to prevent the GDF15-induced loss of QRFPR from the cell surface, without affecting receptor activity, thus providing a potential additional or alternative agent for blocking the GDF15-QRFPR interaction. Thus, agents capable of binding to the extracellular domain of QRFPR represent further potential therapeutic agents for blocking its interaction with GDF15.

Peptides comprising regions of the extracellular domain of CLPTM1 (SEQ ID NO.14) are shown in the Examples below to interact with GDF15 in a ‘pull-down’ assay, and bind to GDF15 with a higher affinity than the peptides comprising regions of QRFPR. Peptides comprising regions of the extracellular domain of CLPTM1 thus represent further therapeutic agents for blocking the interaction between GDF15 and its receptors. The Examples also demonstrates that pre-incubation of an antibody which binds to the extracellular domain of CLPTM1 is capable of inhibiting GSK3b phosphorylation, indicating that it is possible to specifically block the GDF15-CLPTM1 interaction that binds to CLPTM1. Agents capable of binding to the extracellular domain of CLPTM1 represent further potential therapeutic agents for blocking its interaction with GDF15.

It is apparent therefore that GDF15 may exert its physiological effects, including on bone development, cachexia, cancer establishment and metastasis, and reduced immune function at least in part through the two receptors identified in the present application. It would therefore be desirable to block the interaction between GDF15 and these receptors, particularly where levels of GDF15 are elevated, in order to treat or prevent the medical conditions indicated above, or indeed any condition associated with elevated or unwanted GDF15 levels. The present invention addresses this need and accordingly is directed towards the provision of therapeutic agents which inhibit the interaction, or the effect of the interaction, between GDF15 and its receptors.

A first class of therapeutic agents are polypeptides which are based on the ligand binding domains of the GDF15 receptors identified herein. These polypeptides act as ‘decoy’ receptor molecules (“decoy peptides”), and are able to bind to GDF15 free in the circulation, or associated with ECM and/or stroma, and prevent it from binding to the receptors. A second class of therapeutic agents are binding agents which bind to an extracellular domain of the GDF15 receptors, and which thereby act to inhibit the binding of GDF15 to the receptors, and therefore block the interaction between GDF15 and its receptors, or which otherwise inhibit the effect of GDF15 at one or both of the receptors.

Thus, in a first aspect the present invention provides a polypeptide capable of binding to GDF15 and inhibiting its interaction with the receptors—QRFPR and/or CLPTM1, wherein said polypeptide:
(i) has or comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1), or an amino acid sequence having at least 75% sequence identity to SEQ ID NO:5, 12 or 14; or
(ii) is or comprises part of SEQ ID NO:5, 12 or 14, said part comprising at least 6 contiguous amino acids of SEQ ID NO:5, 12 or 14; or
(iii) comprises at least 6 amino acids corresponding to at least 6 contiguous amino acids of SEQ ID NO:5, 12 or 14 and has at least 75% sequence identity to the equivalent amino acid sequence in SEQ ID NO:5, 12 or 14.

A polypeptide of the invention claimed as such does not include native QRFPR or CLPTM1 receptors themselves (more particularly any native or wild-type full length QRFPR or CLPTM1 receptor is excluded, from any species). Accordingly, the polypeptide does not include a polypeptide consisting of, or comprising, SEQ ID NO:1 or 2. Nor does it include polypeptides consisting of or comprising an amino acid sequence representing a full length (i.e. intact or entire) native or wild-type QRFPR or CLPTM1 which is a homologue or orthologue of SEQ ID NO:1 or 2, i.e. a receptor from any other species. Accordingly, a polypeptide of the invention claimed as such does not consist of or comprise SEQ ID NO:287 or SEQ ID NO:288. As discussed below, in a particular embodiment, a polypeptide of the invention claimed as such further is not a polypeptide consisting of the amino acid sequence of any one of SEQ ID NOs: NOs:243-249.

In this regard SEQ ID NOs: 243-245 represent fragments of QRFPR receptors, which are larger than an individual extracellular domain (i.e. they represent receptor fragments which comprise ECD3 or ECD4 of QRFPR) as disclosed as SEQ ID NOs: 2, 4 and 5 of WO 01/87930. Thus, in one embodiment, the polypeptide of the present invention does not include receptor fragments which are larger than ECD3 or ECD4 of QRFPR or larger than the ECD of CLPTM1 (as discussed further below, the ECD of CLPTM1 may or may not include the N-terminal methionine, e.g. as shown in SEQ ID No: 2 or SEQ ID NO:315). WO 01/87930 concerns a receptor termed a “galanin-like GCPR (G-protein coupled receptor)” which appears in one embodiment at least to correspond to QRFPR—the document is focussed on the regulation of binding of galanin to the receptor and does not disclose any interaction of the receptor with GDF15. SEQ ID NOs: 246-249 are fragments of, or polypeptides derived from, two different GPR103 receptor sequences (QRFPR is also known as GPR103) identified in WO 2005/124342 and correspond to SEQ ID NOs: 409, 411, 422 and 426 respectively of WO 2005/124342. SEQ ID NOs; 287 and 288 of the present application correspond to GPR103 receptor sequences identified as SEQ ID NOs: 106 and 107 respectively in WO 2005/124342. This document is concerned with using the receptor sequences, amongst many others, in screens to identify compounds which induce cartilage synthesis in chondrocytes. There is no disclosure in this document of any interaction of GPR103/QRFPR with GDF15. It will be seen therefore that as well as the entire or complete extracellular domains, and fragments, or parts, of the extracellular domains 3 and 4 (ECD3 or 4), of QRFPR or the ECD of CLPTM1, the polypeptide may be or may comprise a polypeptide having an amino acid sequence which is based on, or derived from ECD3 or 4 of QRFPR or the ECD of CLPTM1, or a part thereof, but which is modified with respect to the sequence of the native molecule e.g. by one or more amino acid substitutions, additions and/or deletions. Thus functionally equivalent molecules are included which may have a variant or modified sequence with respect to the native sequence of the ECD or part thereof. By functionally equivalent is meant that the polypeptide retains the ability to bind to GDF15 and inhibit its interaction with the receptor.

In particular, a polypeptide of the invention may comprise at least 6 amino acids and may consist of or comprise a part of an amino acid sequence as set forth in any one of SEQ ID NOs: 5, 12 or 14, said part comprising at least 6 contiguous amino acids, or a part of an amino acid sequence which has at least 75% sequence identity to any one of SEQ ID NOs: 5, 12 or 14, or to a said part. In one embodiment, a part of an amino acid sequence as set forth in any one of SEQ ID NOs: 5, 12 or 14 comprises at least 6 contiguous amino acids and comprises a deletion of at least 2 contiguous or non-contiguous amino acids with respect to SEQ ID NO: 5, 12 or 14. Analogously, a polypeptide which is a part of an amino acid sequence which has at least 75% sequence identity to any one of SEQ ID NO: 5, 12 or 14 may comprise at least 6 contiguous amino acids and a deletion of at least 2 contiguous or non-contiguous amino acids with respect to the functionally equivalent variant sequence corresponding to SEQ ID NO: 5, 12 or 14.

Thus parts of SEQ ID NOs: 5, 12 or 14 may be identified, as described further below, and used as or in polypeptides according to the invention, or polypeptides of the invention may have or comprise an amino acid sequence which has at least 75% sequence identity to any said part, for example as identified below.

Representative or exemplary sequences, representing parts of any one of SEQ ID NOs:5, 12 or 14 are listed below, and include for example SEQ ID NOs:6 to 9 and 11 (parts of SEQ ID NO. 5; ECD3 of QRFPR), SEQ ID NO. 13 (a C-terminal part of SEQ ID NO. 12; ECD4 of QRFPR) and SEQ ID NOs.15 to 18 (parts of SEQ ID NO. 14, the ECD of CLPTM1). A polypeptide of the invention may have or comprise an amino acid sequence which has at least 75% sequence identity to an aforesaid sequence representing a part of SEQ ID NOs:5, 12 or 14. In further embodiments a polypeptide of the invention may have or comprise an amino acid sequence which has at least 75% sequence identity to any sequence representing a part of SEQ ID NOs:5, 12 or 14, as listed below, for example in Table 1 or in any of the examples below.

A further aspect of the present invention provides a binding agent capable of binding to the receptor QRFPR and/or CLPTM1 for use in therapy, wherein said binding agent is capable of inhibiting the interaction between GDF15 and a said receptor.

In particular the polypeptide and/or the binding agent is for use in treating or preventing a condition associated with elevated or unwanted levels of GDF15.

Such conditions are described in more detail below, but in certain preferred embodiments include cancer, cachexia, immunosuppression, and bone disorders.

The binding agent may be any proteinaceous or non-proteinaceous molecule but, as discussed further below, will be preferably be an antibody.

In a preferred embodiment, the binding agent does not bind to a polypeptide of the invention as defined herein. In a further preferred embodiment the binding agent additionally or alternatively does not inhibit binding of an endogenous agonistic ligand to the receptor, particularly wherein the receptor is QRFPR.

A still further aspect of the present invention provides use of a polypeptide of the invention as hereinbefore defined and/or a binding agent of the invention as hereinbefore defined for the manufacture of a medicament for treating or preventing a condition associated with elevated or unwanted levels of GDF15.

Also provided according to the present invention is a pharmaceutical composition comprising a polypeptide of the invention as hereinbefore defined and/or a binding agent of the invention as hereinbefore defined, together with at least one pharmaceutically-acceptable carrier or excipient.

A yet further aspect of the invention provides a kit comprising a polypeptide of the invention as hereinbefore defined and a binding agent of the invention as hereinbefore defined.

The components of the kit may be provided, or formulated, for pharmaceutical delivery (i.e. for therapeutic use) and thus may be provided in the form of pharmaceutical compositions containing the polypeptide or binding agent and one or more pharmaceutically-acceptable carriers or excipients. The components may be formulated or provided for separate administration, including sequentially, or simultaneously. The kit may be for use in in treating or preventing a condition associated with or unwanted levels of GDF15.

Accordingly another aspect of the present invention provides a product comprising a polypeptide of the invention as hereinbefore defined and a binding agent of the invention as hereinbefore defined as a combined preparation for separate, sequential or simultaneous use in treating or preventing a condition associated with or unwanted levels of GDF15.

Also provided is a method of treating or preventing a condition associated with elevated or unwanted levels of GDF15, which method comprises administering to a subject in need thereof an effective amount of a polypeptide of the invention as hereinbefore defined and/or a binding agent of the invention as hereinbefore defined.

The invention may also have non-medical uses and accordingly non-therapeutic methods of inhibiting the interaction of GDF15 with the receptor QRFPR and/or CLPTM1 form a further aspect of the invention. Such a method may involve a contacting a cell or a cell-free system comprising a receptor according to the invention and GDF15 with a polypeptide and/or binding agent according to the invention. Accordingly, in this aspect the invention provides use of the polypeptide of the invention as hereinbefore defined and/or a binding agent of the invention as hereinbefore defined for inhibiting the interaction of GDF15 with the receptor QRFPR and/or CLPTM1 in vitro.

The term “polypeptide” is used broadly herein to include peptide, polypeptide or protein molecules, including any proteinaceous molecule which may include other chemical groups or moieties, e.g. as long as there is a protein/peptide/polypeptide part. As noted above, a polypeptide of the invention comprises at least 6 amino acid residues.

The term “inhibit” includes reducing as well as preventing, and thus includes any effect in reducing, decreasing or lowering the stated activity or property, e.g. the interaction of GDF15 with any one or both of the receptors, or any therapeutic, biological or physiological effect or activity discussed herein. Thus, alternatively expressed, the polypeptide and/or binding agent of the invention may block the interaction of GDF15 with any one or both of the receptors, but this does not necessarily entail or require a complete blocking of receptor binding and/or function, merely a reduction. By way of representative example, binding of GDF15 to the receptor, and/or any effect or aspect of receptor function resulting from or induced or stimulated by such binding, may be reduced by 20, 30, 40, 50, 60 or 70% or more compared to the binding and/or receptor activity or function seen in the absence of the polypeptide and/or binding agent of the invention. Thus, it will be seen that the effect of the GDF15 at the receptor may be inhibited, whether or not binding of GDF15 to the receptor is inhibited. Accordingly, a polypeptide of the invention may inhibit the interaction of GDF15 with the receptor, or the effect of the interaction, with or without inhibiting binding of GDF15 to the receptor.

The term “interaction” includes binding of GDF15 to one or both of the receptors, and/or stimulation or induction of receptor activity or any effect at the receptor. Thus, inhibiting the interaction of GDF15 with the receptor QRFPR and/or CLPTM1 includes inhibiting an effect of (i.e. resulting from) binding of GDF15 to the receptor, without necessarily, as indicated above, inhibiting binding of GDF15 to the receptor.

Binding of GDF15 to a receptor may be assessed or determined using known ligand binding assays as widely described and reported in the literature, including a binding assay as described in the Examples below. The effect of GDF15 binding on a receptor, or more particularly the effect of a polypeptide or binding agent of the invention on GDF15 binding to a receptor may be assessed or determined by determining or assessing any effect resulting from or induced or stimulated by binding of GDF15 and/or a polypeptide or binding agent of the invention to the receptor. Thus for example, binding of GDF15 to QRFPR may cause internalisation or the receptor and/or shedding of the receptor from a cell surface—such effects may be assessed or determined by determining the presence and/or amount of the receptor in exosomes and/or exosomes e.g. by determining the presence or amount of QRFPR-positive exosomes or endosomes, e.g. as described in the Examples below. A polypeptide or binding agent of the invention may cause a decrease in such QRFPR-positive endo- or exosomes.

Any other aspect of receptor activity may also be assessed or determined in the presence of GDF15 and the presence or absence of a polypeptide or binding agent of the invention, e.g. a reduction of the immunosuppressive effect of GDF15 may be assessed or determined by determining the extent or amount of cell-mediated cytotoxicity exhibited by an immune cell (e.g. an NK cell or macrophage) expressing a receptor. Alternatively, other aspects of signalling resulting from receptor stimulation by GDF15 may be assessed and compared in the presence or absence of the binding agent or polypeptide of the invention, e.g. GSK3b phosphorylation.

The term “treating” is used broadly herein to include any aspect of improving or ameliorating a condition or the clinical status of a subject suffering from or having the condition. Thus, a complete cure of the condition is not required and “treating” includes improving any aspect, parameter or symptom of a condition.

Similarly, the term “preventing” is used broadly herein to include any aspect of reducing or delaying a condition, or the onset or progression of a condition. Thus preventing does not require complete or absolute prevention of the development of a condition and may include delaying or slowing the progression or onset of any aspect, symptom or parameter of a condition. The severity of a symptom, parameter or aspect may be reduced and/or it may be delayed in developing. In a particular embodiment, preventing may include preventing (or reducing) metastasis of a cancer, more particularly metastasis of a cancer to bone. In other embodiments the progression or development of one or more symptoms or aspects of a condition may be delayed, or reduced or indeed prevented from developing, e.g. cachexia in a subject suffering from a chronic condition or illness (e.g. cancer, or an inflammatory condition e.g. rheumatoid arthritis) may be prevented from developing or may be delayed and/or reduced in severity.

As noted above, a polypeptide of the invention is based on the extracellular domain(s) (ECDs) of the receptors and can include sequence variants of the native ECDs, as well as fragments thereof (including sequence variants of parts of the ECDs).

Accordingly, alternatively defined, a polypeptide of the invention may be a polypeptide capable of binding to GDF15 and inhibiting its interaction with the receptor QRFPR and/or CLPTM1, wherein said polypeptide has or comprises the sequence

(a) X₁X₂X₃X₄X₅X₆X₇X₈X₉; (Formula I)

wherein:
X₁ is an aromatic amino acid;
X₂ is an aliphatic amino acid;
X₅ is a basic amino acid; and
X₃, X₄, X₆, X₇, X₈ and X₉ may independently be any amino acid (SEQ ID NO:337); or

(b) X₁X₂X₃X₄X₅X₆X₇X₈X₉X₁₀ (Formula II)

wherein
X₁ is an acidic amino acid, e.g. D or E;
X₂ is a basic amino acid e.g. K, R or H, preferably K or R;
X₃ is an acidic amino acid, e.g. D or E;
X₄ is an aromatic amino acid, e.g. F, W or Y;
X₅ is an acidic amino acid, e.g. D or E;
X₆ is an acidic amino acid, e.g. D or E;
X₇ is an aliphatic amino acid, e.g. L, V, I, A or M, preferably L, V or I;
X₈ is A, S or T;
X₉ is an aliphatic amino acid, e.g. L, V, I, A or M, preferably L, V or I; and
X₁₀ is an aromatic amino acid, e.g. R, K or H, preferably R or K (SEQ ID NO:404).

An aromatic amino acid may independently be selected from phenylalanine (F), Tryptophan (W), Tyrosine (Y), tert.-butylglycine, cyclohexylalanine, tert.-butylphenylalanine, biphenylalanine and tri tert.-butyltryptophan.

A basic amino acid may independently be selected from lysine (K), arginine (R), histidine (H), ornithine (Orn), methyllysine (MeK) and acetyllysine (AcK).

An aliphatic amino acid may independently be selected from leucine (L), isoleucine (I), valine (V), alanine (A) methionine (M) and norleucine (Nor).

In a more particular embodiment, in the polypeptide of Formula I
X₁ is F, W or Y;
X₂ is L, V, I, A or M, preferably L, V or I;
X₅ is K, R or H, preferably K or R (SEQ ID NO:338).

Additionally or alternatively in a polypeptide of Formula I
X₃ is an aromatic amino acid, preferably F, W or Y; and/or
X₄ is an acidic amino acid, preferably E or D (SEQ ID NOs:339-344).

Further, in a polypeptide of Formula I additionally or alternatively
X₆ is an acidic amino acid, preferably E or D; and/or
X₇ is a basic amino acid, preferably K, R or H, more preferably K or R (SEQ ID NOs:345-369).

Further, in a polypeptide of Formula I additionally or alternatively
X₈ is an aliphatic amino acid, preferably L, V, I, A or M, more preferably L, V or I; and/or
X₉ is any amino acid, preferably C (SEQ ID NOs:370-402).

In a particular embodiment, a polypeptide of Formula I may have or comprise the sequence X₁X₂X₃X₄X₅X₆X₇X₈X₉ (SEQ ID NO:403), wherein:
X₁ is F, W or Y;
X₂ is L, V or I;
X₃ is F, W or Y;
X₄ is D or E;
X₅ is K, R or H;
X₆ is D or E;
X₇ is K, R or H;
X₈ is L, V or I; and
X₉ is C.

In a further embodiment in a polypeptide of Formula I the sequence X₁X₂X₃X₄X₅X₆X₇X₈X₉ may be flanked on one or both sides by 1 to 10 amino acids, preferably wherein said flanking amino acid sequences comprise amino acids capable of forming an α-helix or a β-sheet.

In such a representative embodiment of a polypeptide of Formula I, residue X₁ is flanked by an amino acid sequence comprising amino acids capable of forming an α-helix and/or residue X₉ is flanked by an amino acid sequence comprising amino acids capable of forming a β-sheet, preferably wherein X₉ is flanked by C.

In a particular embodiment of Formula II X₁ is D or E; X₂ is K, R or H; X₃ is D or E; X₄ is F, W or Y; X₅ is D or E; X₆ is D or E; X₇ is L, V or I; X₈ is A, S or T; X₉ is L, V or I; and X₁₀ is R, K or H (SEQ ID NO:405).

In a particular embodiment of any aspect of a polypeptide according to the present invention, the polypeptide may comprise the sequence as set forth in SEQ ID NO:210 (FLYEK).

In a further embodiment of any aspect of a polypeptide according to the present invention, the polypeptide may comprise residues the sequence as set forth in SEQ ID NO. 211 (corresponding to residues 255-257 of SEQ ID NO:1 WTS).

As noted above, in representative embodiments a polypeptide of the invention may have or comprise a sequence representing or corresponding to part of any one of the ECD sequences of SEQ ID NOs, 5, 12 or 14. Such a sequence may have at least 75% sequence identity to a part of any one of the amino acid sequences of SEQ ID NO. 5, 12 or 14, including for example any one of SEQ ID NO:11, SEQ ID NO. 7 or SEQ ID NOs:6, 8, or 9, representing sequences derived from ECD3 of QRFPR; SEQ ID NO. 13, representing a sequence derived from ECD4 of QRFPR; SEQ ID NO:17, SEQ ID NO 18, SEQ ID NO:15 (YISEHEH), or SEQ ID NO: 16 (LFWEQH), representing sequences derived from the ECD of CLPTM1.

In a further embodiment, the polypeptide of the present invention may comprises a sequence as set forth in SEQ ID NO:15 (YISEHEH) or a sequence having at least 75% sequence identity thereto and a sequence as set forth in SEQ ID NO:16 (LFWEQH) or a sequence having at least 75% sequence identity thereto.

Other representative polypeptides according to the present invention may have or comprise an amino acid sequence as set out in Table 1 below, or a sequence having at least 75% sequence identity to any such sequence, or, where applicable, a sequence which is a part of any such sequence, wherein said part comprises at least 6 contiguous amino acids.

TABLE 1

| Sequence number | Polypeptide sequence |
|---|---|
| SEQ ID NO: 20 | QQLEIKYDFLYEKEHICCLEEWTSPVHQ |
| SEQ ID NO: 21 | QQLEIKYDFLYEKEHICCLEEWTSPVH |
| SEQ ID NO: 22 | QQLEIKYDFLYEKEHICCLEEWTSPV |
| SEQ ID NO: 23 | QQLEIKYDFLYEKEHICCLEEWTSP |
| SEQ ID NO: 24 | QQLEIKYDFLYEKEHICCLEEWTS |
| SEQ ID NO: 25 | QQLEIKYDFLYEKEHICCLEEWT |
| SEQ ID NO: 26 | QQLEIKYDFLYEKEHICCLEEW |
| SEQ ID NO: 27 | QQLEIKYDFLYEKEHICCLEE |
| SEQ ID NO: 28 | QQLEIKYDFLYEKEHICCLE |
| SEQ ID NO: 29 | QQLEIKYDFLYEKEHICCL |
| SEQ ID NO: 30 | QQLEIKYDFLYEKEHICC |
| SEQ ID NO: 31 | QQLEIKYDFLYEKEHIC |
| SEQ ID NO: 32 | QLEIKYDFLYEKEHICC |
| SEQ ID NO: 33 | LEIKYDFLYEKEHICCL |
| SEQ ID NO: 34 | EIKYDFLYEKEHICCLE |
| SEQ ID NO: 35 | IKYDFLYEKEHICCLEE |
| SEQ ID NO: 36 | KYDFLYEKEHICCLEEW |
| SEQ ID NO: 37 | YDFLYEKEHICCLEEWT |
| SEQ ID NO: 38 | DFLYEKEHICCLEEWTS |
| SEQ ID NO: 39 | QLEIKYDFLYEKEHI |
| SEQ ID NO: 40 | LEIKYDFLYEKEHIC |
| SEQ ID NO: 41 | EIKYDFLYEKEHICC |
| SEQ ID NO: 42 | IKYDFLYEKEHICCL |
| SEQ ID NO: 43 | KYDFLYEKEHICCLE |
| SEQ ID NO: 44 | YDFLYEKEHICCLEE |
| SEQ ID NO: 45 | DFLYEKEHICCLEEW |
| SEQ ID NO: 46 | QLEIKYDFLYEKEHIC |
| SEQ ID NO: 47 | LEIKYDFLYEKEHIC |
| SEQ ID NO: 48 | EIKYDFLYEKEHIC |
| SEQ ID NO: 49 | IKYDFLYEKEHIC |
| SEQ ID NO: 50 | KYDFLYEKEHIC |
| SEQ ID NO: 51 | YDFLYEKEHIC |
| SEQ ID NO: 52 | DFLYEKEHIC |
| SEQ ID NO: 53 | QLEIKYDFLYEKEHICCLEEWTSPVHQK |
| SEQ ID NO: 54 | LEIKYDFLYEKEHICCLEEWTSPVHQK |
| SEQ ID NO: 55 | EIKYDFLYEKEHICCLEEWTSPVHQK |
| SEQ ID NO: 56 | IKYDFLYEKEHICCLEEWTSPVHQK |
| SEQ ID NO: 57 | KYDFLYEKEHICCLEEWTSPVHQK |
| SEQ ID NO: 58 | YDFLYEKEHICCLEEWTSPVHQK |
| SEQ ID NO: 59 | DFLYEKEHICCLEEWTSPVHQK |
| SEQ ID NO: 60 | FLYEKEHICCLEEWTSPVHQK |
| SEQ ID NO: 61 | DFLYEKEHICCLEEWTSPVHQ |
| SEQ ID NO: 62 | YDFLYEKEHICCLEEWTSPVH |
| SEQ ID NO: 63 | KYDFLYEKEHICCLEEWTSPV |
| SEQ ID NO: 64 | IKYDFLYEKEHICCLEEWTSP |
| SEQ ID NO: 65 | EIKYDFLYEKEHICCLEEWTS |
| SEQ ID NO: 66 | LEIKYDFLYEKEHICCLEEWT |
| SEQ ID NO: 67 | QLEIKYDFLYEKEHICCLEEW |
| SEQ ID NO: 68 | FLYEKEHICCLEEWTSPVHQ |
| SEQ ID NO: 69 | FLYEKEHICCLEEWTSPVH |
| SEQ ID NO: 70 | FLYEKEHICCLEEWTSPV |
| SEQ ID NO: 71 | FLYEKEHICCLEEWTSP |
| SEQ ID NO: 72 | FLYEKEHICCLEEWTS |
| SEQ ID NO: 73 | FLYEKEHICCLEEWT |
| SEQ ID NO: 74 | FLYEKEHICCLEEW |
| SEQ ID NO: 75 | FLYEKEHICCLEE |
| SEQ ID NO: 76 | FLYEKEHICCLE |
| SEQ ID NO: 77 | FLYEKEHICCL |
| SEQ ID NO: 78 | FLYEKEH ICC |
| SEQ ID NO: 79 | MMIEYSNFEKEYDDVTIK |
| SEQ ID NO: 80 | MIEYSNFEKEYDDVTIK |
| SEQ ID NO: 81 | IEYSNFEKEYDDVTIK |
| SEQ ID NO: 82 | EYSNFEKEYDDVTIK |
| SEQ ID NO: 83 | YSNFEKEYDDVTIK |
| SEQ ID NO: 84 | SNFEKEYDDVTIK |
| SEQ ID NO: 85 | NFEKEYDDVTIK |
| SEQ ID NO: 86 | FEKEYDDVTIK |
| SEQ ID NO: 87 | YISEHEHFTDFNATSALFWEQHDLVYGDWT |
| SEQ ID NO: 88 | YISEHEHFTDFNATSALFWEQHDLVYGDW |
| SEQ ID NO: 89 | YISEHEHFTDFNATSALFWEQHDLVYGD |
| SEQ ID NO: 90 | YISEHEHFTDFNATSALFWEQHDLVYG |
| SEQ ID NO: 91 | YISEHEHFTDFNATSALFWEQHDLVY |
| SEQ ID NO: 92 | YISEHEHFTDFNATSALFWEQHDLV |
| SEQ ID NO: 93 | YISEHEHFTDFNATSALFWEQHDL |
| SEQ ID NO: 94 | YISEHEHFTDFNATSALFWEQHD |
| SEQ ID NO: 95 | YISEHEHFTDFNATSALFWEQH |
| SEQ ID NO: 96 | YISEHEHFTDFNATSALFWEQ |
| SEQ ID NO: 97 | YISEHEHFTDFNATSALFWE |
| SEQ ID NO: 98 | YISEHEHFTDFNATSALFW |
| SEQ ID NO: 99 | YISEHEHFTDFNATSALF |
| SEQ ID NO: 100 | YISEHEHFTDFNATSAL |
| SEQ ID NO: 101 | YISEHEHFTDFNATSA |
| SEQ ID NO: 102 | YISEHEHFTDFNATS |
| SEQ ID NO: 103 | YISEHEHFTDFNAT |
| SEQ ID NO: 104 | YISEHEHFTDFNA |
| SEQ ID NO: 105 | YISEHEHFTDFN |
| SEQ ID NO: 106 | YISEHEHFTDF |
| SEQ ID NO: 107 | YISEHEHFTD |
| SEQ ID NO: 108 | YISEHEHFT |
| SEQ ID NO: 109 | YISEHEHF |
| SEQ ID NO: 110 | ISEHEHFTDFNATSALFWEQHDLVYGDWTS |
| SEQ ID NO: 111 | SEHEHFTDFNATSALFWEQHDLVYGDWTS |
| SEQ ID NO: 112 | EHEHFTDFNATSALFWEQHDLVYGDWTS |
| SEQ ID NO: 113 | HEHFTDFNATSALFWEQHDLVYGDWTS |
| SEQ ID NO: 114 | EHFTDFNATSALFWEQHDLVYGDWTS |
| SEQ ID NO: 115 | HFTDFNATSALFWEQHDLVYGDWTS |
| SEQ ID NO: 116 | FTDFNATSALFWEQHDLVYGDWTS |
| SEQ ID NO: 117 | TDFNATSALFWEQHDLVYGDWTS |
| SEQ ID NO: 118 | DFNATSALFWEQHDLVYGDWTS |
| SEQ ID NO: 119 | FNATSALFWEQHDLVYGDWTS |
| SEQ ID NO: 120 | NATSALFWEQHDLVYGDWTS |
| SEQ ID NO: 121 | ATSALFWEQHDLVYGDWTS |
| SEQ ID NO: 122 | TSALFWEQHDLVYGDWTS |
| SEQ ID NO: 123 | SALFWEQHDLVYGDWTS |
| SEQ ID NO: 124 | ALFWEQHDLVYGDWTS |
| SEQ ID NO: 125 | LFWEQHDLVYGDWTS |
| SEQ ID NO: 126 | LFWEQHDLVYGDWT |
| SEQ ID NO: 127 | LFWEQHDLVYGDW |
| SEQ ID NO: 128 | LFWEQHDLVYGD |
| SEQ ID NO: 129 | LFWEQHDLVYG |
| SEQ ID NO: 130 | LFWEQHDLVY |
| SEQ ID NO: 131 | LFWEQHDLV |
| SEQ ID NO: 132 | LFWEQHDL |
| SEQ ID NO: 133 | LFWEQHD |
| SEQ ID NO: 134 | FTDFNATSALFWEQH |
| SEQ ID NO: 135 | TDFNATSALFWEQHD |
| SEQ ID NO: 136 | DFNATSALFWEQHDL |
| SEQ ID NO: 137 | FNATSALFWEQHDLV |
| SEQ ID NO: 138 | NATSALFWEQHDLVY |
| SEQ ID NO: 139 | ATSALFWEQHDLVYG |
| SEQ ID NO: 140 | TSALFWEQHDLVYGD |
| SEQ ID NO: 141 | SALFWEQHDLVYGDW |
| SEQ ID NO: 142 | ALFWEQHDLVYGDWT |
| SEQ ID NO: 143 | ISEHEHFTDFNATSALFWEQH |
| SEQ ID NO: 144 | SEHEHFTDFNATSALFWEQH |
| SEQ ID NO: 145 | EHEHFTDFNATSALFWEQH |
| SEQ ID NO: 146 | HEHFTDFNATSALFWEQH |
| SEQ ID NO: 147 | EHFTDFNATSALFWEQH |
| SEQ ID NO: 148 | HFTDFNATSALFWEQH |
| SEQ ID NO: 149 | FTDFNATSALFWEQH |
| SEQ ID NO: 150 | TDFNATSALFWEQH |
| SEQ ID NO: 151 | DFNATSALFWEQH |
| SEQ ID NO: 152 | FNATSALFWEQH |
| SEQ ID NO: 153 | NATSALFWEQH |
| SEQ ID NO: 154 | ATSALFWEQH |
| SEQ ID NO: 155 | SALFWEQH |
| SEQ ID NO: 156 | ALFWEQH |
| SEQ ID NO: 157 | ISEHEHFTDFNATSALFWEQHD |
| SEQ ID NO: 158 | SEHEHFTDFNATSALFWEQHDL |
| SEQ ID NO: 159 | EHEHFTDFNATSALFWEQHDLV |
| SEQ ID NO: 160 | HEHFTDFNATSALFWEQHDLVY |
| SEQ ID NO: 161 | EHFTDFNATSALFWEQHDLVYG |
| SEQ ID NO: 162 | HFTDFNATSALFWEQHDLVYGD |
| SEQ ID NO: 163 | FTDFNATSALFWEQHDLVYGDW |
| SEQ ID NO: 164 | TDFNATSALFWEQHDLVYGDWT |
| SEQ ID NO: 165 | DFNATSALFWEQHDLVYGDWTS |
| SEQ ID NO: 166 | SALFWEQHDLVYGDW |
| SEQ ID NO: 167 | TSALFWEQHDLVYGD |
| SEQ ID NO: 168 | ATSALFWEQHDLVYG |
| SEQ ID NO: 169 | NATSALFWEQHDLVY |
| SEQ ID NO: 170 | FNATSALFWEQHDLV |
| SEQ ID NO: 171 | DFNATSALFWEQHDL |
| SEQ ID NO: 172 | TDFNATSALFWEQHD |
| SEQ ID NO: 173 | FTDFNATSALFWEQH |
| SEQ ID NO: 174 | GIEYSNFEKEYDDVTIK |
| SEQ ID NO: 175 | QQLEIKYDFLYEKEHI |
| SEQ ID NO: 176 | QQLEIKYDFLYEKEH |
| SEQ ID NO: 177 | QLEIKYDFLYEKEH |
| SEQ ID NO: 178 | LEIKYDFLYEKEHICC |
| SEQ ID NO: 179 | LEIKYDFLYEKEHI |
| SEQ ID NO: 180 | LEIKYDFLYEKEH |
| SEQ ID NO: 181 | QRLEIKYDFLYEKEHICC |
| SEQ ID NO: 182 | QRLEIKYDFLYEKEHIC |
| SEQ ID NO: 183 | QRLEIKYDFLYEKEHI |
| SEQ ID NO: 184 | QRLEIKYDFLYEKEH |
| SEQ ID NO: 185 | RLEIKYDFLYEKEHICC |
| SEQ ID NO: 186 | LEIKYDFLYEKEHICC |
| SEQ ID NO: 187 | RLEIKYDFLYEKEHIC |
| SEQ ID NO: 188 | RLEIKYDFLYEKEHI |
| SEQ ID NO: 189 | RLEIKYDFLYEKEH |
| SEQ ID NO: 190 | SALFWEQHDLVYGDWT |
| SEQ ID NO: 191 | FNATSALFWEQHDLVYGDWTSGEN |
| SEQ ID NO: 192 | FNATSALFWEQHDLVYGDWTSGE |
| SEQ ID NO: 193 | FNATSALFWEQHDLVYGDWTSG |
| SEQ ID NO: 194 | FNATSALFWEQHDLVYGDWTS |
| SEQ ID NO: 195 | FNATSALFWEQHDLVYGDWT |
| SEQ ID NO: 196 | NATSALFWEQHDLVYGDWTSGEN |
| SEQ ID NO: 197 | ATSALFWEQHDLVYGDWTSGEN |
| SEQ ID NO: 198 | TSALFWEQHDLVYGDWTSGEN |
| SEQ ID NO: 199 | SALFWEQHDLVYGDWTSGEN |
| SEQ ID NO: 200 | YFNDYWNLQ |
| SEQ ID NO: 201 | YPIIYFNDYWNLQKDYY |
| SEQ ID NO: 202 | YPIIYFNDYWNLQKDY |
| SEQ ID NO: 203 | YPIIYFNDYWNLQKD |
| SEQ ID NO: 204 | YPIIYFNDYWNLQK |
| SEQ ID NO: 205 | YPIIYFNDYWNLQ |
| SEQ ID NO: 206 | PIIYFNDYWNLQKDYY |
| SEQ ID NO: 207 | IlYFNDYWNLQKDYY |
| SEQ ID NO: 208 | IYFNDYWNLQKDYY |
| SEQ ID NO: 209 | YFNDYWNLQKDYY |
| SEQ ID NO: 210 | FLYEK |
| SEQ ID NO: 211 | WTS |
| SEQ ID NO: 212 | PKDT |
| SEQ ID NO: 213 | APRVASRNLFPKDT |
| SEQ ID NO: 214 | PRVASRNLFPKDT |
| SEQ ID NO: 215 | RVASRNLFPKDT |
| SEQ ID NO: 216 | VASRNLFPKDT |
| SEQ ID NO: 217 | ASRNLFPKDT |
| SEQ ID NO: 218 | SRNLFPKDT |
| SEQ ID NO: 219 | RNLFPKDT |
| SEQ ID NO: 220 | NLFPKDT |
| SEQ ID NO: 221 | LFPKDT |
| SEQ ID NO: 222 | FPKDT |
| SEQ ID NO: 223 | PRVASRNLFPKDTL |
| SEQ ID NO: 224 | VASRNLFPKDTLM |
| SEQ ID NO: 225 | ASRNLFPKDTLMN |
| SEQ ID NO: 226 | SRNLFPKDTLMNL |
| SEQ ID NO: 227 | RNLFPKDTLMNLH |
| SEQ ID NO: 228 | NLFPK DTLMNLHV |
| SEQ ID NO: 229 | LFPKDTLMNLHVY |
| SEQ ID NO: 230 | FPKDTLMNLHVYI |
| SEQ ID NO: 231 | PKDTLMNLHVYIS |
| SEQ ID NO: 232 | PKDTLMNLHVYI |
| SEQ ID NO: 233 | PKDTLMNLHVY |
| SEQ ID NO: 234 | PKDTLMNLHV |
| SEQ ID NO: 235 | PKDTLMNLH |
| SEQ ID NO: 236 | PKDTLMNL |
| SEQ ID NO: 237 | PKDTLMN |
| SEQ ID NO: 238 | PKDTLM |
| SEQ ID NO: 239 | PKDTL |
| SEQ ID NO: 250 | AAAQEADGARSAVVAAGGGSG |
| SEQ ID NO: 251 | SGQVTSNGSIGRDPPAETQPG |
| SEQ ID NO: 252 | QNPPAQPAPNAWQVIKGVLFG |
| SEQ ID NO: 253 | RIFIIWAISSWFRRGPAPQDG |
| SEQ ID NO: 254 | QAGPGGAPRVASRNLFPKDTG |
| SEQ ID NO: 255 | LMNLHVYISEHEHFTDFNATG |
| SEQ ID NO: 256 | SALFWEQHDLVYGDWTSGENG |
| SEQ ID NO: 257 | SDGCYEHFAELDIPQSVQQNG |
| SEQ ID NO: 258 | GSIYIHVYFTKSGFHPDPRQG |
| SEQ ID NO: 259 | KALYRRLATVHMSRMINKYKG |
| SEQ ID NO: 260 | RRRFQKTKNLLTGETEADPEG |
| SEQ ID NO: 261 | MIKRAEDYGPVEVISHWHPNG |
| SEQ ID NO: 262 | ITINIVDDHTPWVKGSVPPPG |
| SEQ ID NO: 263 | LDQYVKFDAVSGDYYPIIYFG |
| SEQ ID NO: 264 | NDYWNLQKDYYPINESLASLG |
| SEQ ID NO: 265 | PLRVSFCPLSLWRWQLYAAQG |
| SEQ ID NO: 266 | STKSPWNFLGDELYEQSDEEG |
| SEQ ID NO: 267 | YEQSDEEQDSVKVALLETNPG |
| SEQ ID NO: 268 | LWRWQLYAAQSTKSPWNFLGG |
| SEQ ID NO: 269 | YPINESLASLPLRVSFCPLSG |
| SEQ ID NO: 270 | GDYYPIIYFNDYWNLQKDYYG |
| SEQ ID NO: 271 | WQVIKGVLFRIFIIWAISSWG |
| SEQ ID NO: 272 | RNLFPKDTLMNLHVYISEHEG |
| SEQ ID NO: 273 | FTDFNATSALFWEQHDLVYGG |
| SEQ ID NO: 289 | AAAQEADGARSAVVAAGGGS |
| SEQ ID NO: 290 | SGQVTSNGSIGRDPPAETQP |
| SEQ ID NO: 291 | QNPPAQPAPNAWQVIKGVLF |
| SEQ ID NO: 292 | RIFIIWAISSWFRRGPAPQD |
| SEQ ID NO: 293 | QAGPGGAPRVASRNLFPKDT |
| SEQ ID NO: 294 | LMNLHVYISEHEHFTDFNAT |
| SEQ ID NO: 295 | SALFWEQHDLVYGDWTSGEN |
| SEQ ID NO: 296 | SDGCYEHFAELDIPQSVQQN |
| SEQ ID NO: 297 | GSIYIHVYFTKSGFHPDPRQ |
| SEQ ID NO: 298 | KALYRRLATVHMSRMINKYK |
| SEQ ID NO: 299 | RRRFQKTKNLLTGETEADPE |
| SEQ ID NO: 300 | MIKRAEDYGPVEVISHWHPN |
| SEQ ID NO: 301 | ITINIVDDHTPWVKGSVPPP |
| SEQ ID NO: 302 | LDQYVKFDAVSGDYYPIIYF |
| SEQ ID NO: 303 | NDYWNLQKDYYPINESLASL |
| SEQ ID NO: 304 | PLRVSFCPLSLWRWQLYAAQ |
| SEQ ID NO: 305 | STKSPWNFLGDELYEQSDEE |
| SEQ ID NO: 306 | YEQSDEEQDSVKVALLETNP |
| SEQ ID NO: 307 | LWRWQLYAAQSTKSPWNFLG |
| SEQ ID NO: 308 | YPINESLASLPLRVSFCPLS |
| SEQ ID NO: 309 | GDYYPIIYFNDYWNLQKDYY |
| SEQ ID NO: 310 | WQVIKGVLFRIFIIWAISSW |
| SEQ ID NO: 311 | RNLFPKDTLMNLHVYISEHE |
| SEQ ID NO: 312 | FTDFNATSALFWEQHDLVYG |
| SEQ ID NO: 316 | QLEIKYDFLYEKEHICCLEE |
| SEQ ID NO: 317 | QLEIKYDFLYEKEHICCLE |
| SEQ ID NO: 318 | QLEIKYDFLYEKEHICCL |

The present invention accordingly provides therapeutic agents (binding agents and polypeptides) which are capable of reducing the activity, or the effect, of GDF15 in a subject by blocking the interaction between GDF15 and its receptors. Such agents are selected from the group consisting of portions, particularly soluble portions, of the extracellular domains of the GDF15 receptors QRFPR or CLPTM1, or polypeptides which are based on, or derived, from the ECDs or portions thereof, and binding agents which may specifically bind to the extracellular domain of one of these GDF15 receptors.

A polypeptide having a high degree of sequence identity with a portion of an extracellular domain of QRFPR or CLPTM1 may be said to be ‘based on’ or ‘derived from’ or to have or comprise a sequence that is ‘based on’ or ‘derived from’ an extracellular domain of a GDF15 receptor. Such polypeptides may thus comprise or have a sequence which corresponds to an ECD or part thereof, but which includes some sequence variation or modification, as compared to the native human ECD sequences as set out in SEQ ID NO. 5, 12 or 14. A “corresponding” sequence may thus be correlated to, or aligned with, a sequence contained within an ECD of SEQ ID NO. 5, 12, or 14 (i.e. to or with a part of a said sequence), but may contain one more sequence variations with respect to a said sequence.

A polypeptide of the invention may include variants, or homologues, obtained or derived from QRFPR or CLPTM1 receptors from other species. Thus the polypeptide may be derived from or based on the ECDs of the equivalent or corresponding receptors from other species, particularly other mammalian species, e.g. dog or mouse.

Both of the receptors identified in the present invention are transmembrane proteins, and thus in preferred embodiments of the various aspects of the invention the polypeptides described herein represent or comprise an extracellular part or portion of the extracellular domain or domains of the proteins. Accordingly, the polypeptide preferably is not, or does not comprise, the full length protein, and particularly is not, or does not comprise, the full-length wild-type or native receptor protein, e.g. the polypeptide does not have (or consist of) the sequence set forth in SEQ ID NOs:1 or 2, or an equivalent sequence from another species, e.g. mouse or rat etc. In other embodiments, the polypeptide does not have, or does not comprise, the sequence set forth in SEQ ID NO:287 or SEQ ID NO:288

In certain preferred embodiments, the polypeptides do not comprise amino acid sequences derived or obtained from the receptor (i.e. amino acid sequences corresponding to receptor sequences) that do not form part of the extracellular domains of the respective receptor proteins, i.e. which amino acid sequences which form the transmembrane and/or intracellular domains of the respective receptor proteins. Specifically, in preferred embodiments, polypeptides derived from QRFPR may not comprise the residues (amino acid sequences)N-terminal and/or C-terminal to the extracellular domains of QRFPR, or part thereof, and in particular the residues (amino acid sequences) N-terminal and/or C-terminal to ECDs 3 and/or 4 of QRFPR (i.e. the residues/sequences within SEQ ID NO:1 that are N-terminal or C-terminal to the sequences represented by SEQ ID NOs:5 and 12 or part thereof), and polypeptides derived from CLPTM1 may not comprise the residues (amino acid sequences) C-terminal to the extracellular domain of CLPTM1, or part thereof (i.e. residues/sequences within SEQ ID NO:2 that are C-terminal to the sequence represented by SEQ ID NO:14 or part thereof). Thus, in certain embodiments, the polypeptide does not comprise amino acids 1-183, 213-292 or 312-431 of SEQ ID NO:1 (for polypeptides derived from QRFPR). In other embodiments, the polypeptide does not comprise amino acids 355-669 of SEQ ID NO:2 (for polypeptides derived from CLPTM1).

In certain embodiments of any of the various aspects of the invention, the polypeptides do not consist of an amino acid sequence representing an entire native extracellular domain. That is, in such embodiments the polypeptide does not consist of ECD3 and ECD4 of QRFPR, or the ECD of CLPTM1. Accordingly in one such embodiment the polypeptide does not consist of the sequence of any one of SEQ ID NOs: 5, 12, or 14. In other embodiments the polypeptide may comprise an entire native extracellular domain together with one or more additional amino acid sequences which are not derived from, or do not correspond to, QRFPR or CLPTM1 sequences, In other words the polypeptide may consist of ECD3 or ECD4 of QRFPR and/or the ECD of CLPTM1 (or indeed a part of any such ECD) together with one or more amino acid sequences which are not sequences from a native QRFPR or CLPTM1 receptor, or more particularly which are not sequences which flank, or are immediately adjacent to, the respective ECDs in the native receptor. The additional amino acid sequence may in this respect comprise 2 or more amino acids, It will be seen that in such embodiments the polypeptides do not contain any amino acid sequences derived from or corresponding to a native/wild-type QRFPR or CLPTM1 receptor, other than the sequences derived from or corresponding to ECD3 and/or ECD4 of QRFPR or a part thereof and/or the ECD of CLPTM1 or a part thereof.

Accordingly, in certain embodiments, wherein the polypeptide comprises an ECD or a part thereof as part of a longer amino acid sequence, the sequence of the ECD or part thereof may be comprised in a “non-native” sequence context; i.e. the sequence flanking on one or both sides the amino acid sequence which corresponds to the ECD or part thereof is not a sequence which flanks the sequence of the ECD or part thereof (e.g. SEQ ID NO:5, 12 or 14 or a part thereof) in the native full length receptor from which the ECD or part thereof is obtained or derived, or to which it corresponds (e.g. in SEQ ID NO.1 (full-length QRFPR) or CLPTM1 (full-length CLPTM1)). Thus, in certain embodiments, at least 2, 3, 4, 5, 6 or 7 contiguous amino acids flanking (N-terminal and/or C-terminal to) SEQ ID NO:5 or 12 are not from SEQ ID NO:1. In certain other embodiments, at least 2, 3, 4, 5, 6 or 7 contiguous amino acids flanking (C-terminal) to SEQ ID NO:14 are not from SEQ ID NO:2. It will be understood by this that the flanking amino acids are those immediately adjacent to the extracellular domains defined by SEQ ID NOs:5, 12 in SEQ ID NO:1, or SEQ ID NO:14 in SEQ ID NO:2.

In yet further embodiments of any of the various aspects of the invention, the polypeptide of the invention may be (i.e. may consist of) or may comprise a part of ECD3 and/or ECD4 of QRFPR and/or the ECD of CLPTM1. In particular embodiments such a part may represent a deletion of at least 2 contiguous or non-contiguous amino acids from the ECD. Thus, in representative embodiments a polypeptide according to the invention may consist of or may comprise an amino acid sequence corresponding to, or represented by, SEQ ID NOs:5, 12 or 14 with a deletion of more than one (i.e. 2, 3, 4, 5, 6, 7 or more) amino acids (which may be contiguous or non-contiguous). In other words, the deleted amino acids may be found at the N-terminus and/or C-terminus of the SEQ ID NO: 5, 12 or 14.

As noted above, inn certain embodiments, the polypeptide sequence is not any of SEQ ID NOs:243-249. In yet further embodiments, the polypeptide sequence is not SEQ ID NO:287 or SEQ ID NO:288.

Accordingly, in one particular embodiment the invention provides a polypeptide capable of binding to GDF15 and inhibiting its interaction with the receptors—QRFPR and/or CLPTM1, wherein said polypeptide:
(i) comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1) wherein the amino acid sequence is not flanked at either or both ends by an amino acid sequence which flanks the said amino acid sequence in a native QRFPR or CLPTM1 receptor, or comprises an amino acid sequence which is not the amino acid sequence of SEQ ID NOs: 5, 12, or 14 but which has at least 75% sequence identity to SEQ ID NO:5, 12 or 14, wherein optionally the amino acid sequence is not flanked at either or both ends by an amino acid sequence which flanks the said amino acid sequence in a native QRFPR or CLPTM1 receptor; or
(ii) is or comprises a part of SEQ ID NO:5, 12 or 14, said part comprising at least 6 contiguous amino acids of SEQ ID NO:5, 12 or 14, wherein (i) at least 2 contiguous or non-contiguous amino acids of SEQ ID NO:5, 12 or 14 are deleted and/or (ii) said part is not flanked at either or both ends by an amino acid sequence which flanks the said amino acid sequence in a native QRFPR or CLPTM1 receptor; or
(iii) comprises an amino acid sequence having at least 6 amino acids corresponding to at least 6 contiguous amino acids of SEQ ID NO:5, 12 or 14 and has at least 75% sequence identity to the equivalent amino acid sequence in SEQ ID NO:5, 12 or 14, wherein (i) at least 2 contiguous or non-contiguous amino acids of SEQ ID NO:5, 12 or 14 are deleted and/or (ii) the amino acid sequence corresponding to SEQ ID NO:5, 12 or 14 is not flanked at either or both ends by an amino acid sequence which flanks the said amino acid sequence in a native QRFPR or CLPTM1 receptor.

As noted above, peptides comprising sequences derived from extracellular domains 3 and 4 of QRFPR have been found to interact with GDF15 in vitro, and data indicating that such peptides are not only capable of binding to GDF15, but are also capable of blocking the interaction of QRFPR and GDF15, are provided in the Examples below.

Polypeptides having or comprising a sequence as set out in Formula I or Formula II above may represent polypeptides derived from or based on ECD3 and ECD4 of QRFPR respectively.

As noted above, GDF15 has been found to bind to both extracellular domains 3 and 4 of QRFPR simultaneously. The binding site in QRFPR for GDF15 is thus thought to comprise a portion of both extracellular domains 3 and 4 of QRFPR. Accordingly, a polypeptide according to the present invention may comprise sequences derived from both extracellular domains 3 and 4 of QRFPR, including a first sequence as set forth in any one of SEQ ID NOs 5, 6 to 9, or 11, or a sequence having at least 75% sequence identity thereto, and a second sequence as set forth in SEQ ID NO:12 or 13 or a sequence having at least 75% sequence identity thereto. In a further particular embodiment said first and second sequences are separated by a linker. They may be present in either order. In a preferred embodiment, the polypeptide may include a first sequence as set forth in SEQ ID NO:178 or 179, and second sequence as set forth in SEQ ID NO:12.

GDF15 acts to down-regulate the expression of QRFPR at the cell surface by inducing shedding of exosomes containing QRFPR, or by inducing the internalisation of QRFPR. However, QRFPR has other ligands (for example P518 and RF-amide 43) which have an agonistic effect and induce signalling through QRFPR. In a preferred embodiment of the present invention, a polypeptide having or comprising a sequence derived from an extracellular domain of QRFPR will not bind to a QRFPR agonist, and will not reduce the ability of a QRFPR agonist to activate signalling through QRFPR.

Polypeptides comprising sequences derived from the extracellular domain of CLPTM1 have also been found to interact with GDF15, and may be used to block the interaction between GDF15 and its receptors. In a particular embodiment such a polypeptide may have or comprise a sequence as set forth in SEQ ID NO.17 or 18. It is believed, that similarly to QRFPR, receptor CLPTM1 may comprise two binding sites for GDF15, namely that GDF15 may have at least two contact points within the CLPTM1 receptor, or more particularly the ECD thereof. In particular, we presently believe, based on our studies reported in the Examples below, that two binding sites, or contact points, for GDF15 may lie in SEQ ID NO.17, representing amino acids 108 to 138 of SEQ ID NO. 14 (the ECD of human CLPTM1). These are provided as SEQ ID NOs:15 (YISEHEH) and 16 (LFWEQH), which represent or comprise or constitute a part of these binding sites. A representative polypeptide of the invention may comprise a sequence corresponding to, or based on or derived from one or both of these sequences. A representative polypeptide including both binding sites has or comprises SEQ ID NO. 18 or a sequence having at least 75% sequence identity thereto. Representative polypeptides of the invention also include a polypeptide having or comprising a sequence as set forth in SEQ NO:18, representing amino acids 123 to 138 of SE ID NO:14, which includes one putative binding site.

Further experiments have suggested that GDF15 may have other or additional binding sites in CLPTM1 (see e.g. Example 19). Thus, one such additional binding site may be represented by or may comprise SEQ ID NO:200 (YFNDYWNLQ). A representative polypeptide of the invention may comprise a sequence corresponding to, or based on or derived from this sequence. A representative polypeptide accordingly has or comprises SEQ ID NO. 200, or a sequence having at least 75% sequence identity thereto, for example the polypeptide may have or comprise any one of SEQ ID NOs; 201 to 209, or a sequence having at least 75% sequence identity thereto.

In one embodiment, a polypeptide obtained or derived from CLPTM1 does not inhibit binding of an endogenous ligand to CLPTM1, and/or does not inhibit receptor activation or stimulation by an endogenous ligand. However, in other embodiments it may be desirable or advantageous for binding and/or receptor activation or stimulation by an endogenous ligand.

Furthermore, a polypeptide of the invention may comprise an amino acid sequence obtained or derived from QRFPR, including any such sequence described herein and an amino acid sequence obtained or derived from CLPTM1, including any such sequence described herein.

It will be seen that the invention provides a range of different polypeptides which may be obtained or derived from different receptors, and/or different ECDs and may have different sizes and which may contain one or more binding sites for GDF15. Accordingly a repertoire, or panel, or library, or set of different polypeptides may be provided. Different polypeptides may differ in their affinity for GDF15 and may be used according to choice, e.g. to provide a differing binding activities for GDF15. They may be used singly or in combination. Thus, different polypeptides may allow therapy to be extended, or continued, for example if the subject being treated develops an immune response to a particular polypeptide, another may then be selected for use. Polypeptides may be used in combination to provide a greater therapeutic effect.

As noted above, a polypeptide of the invention comprises a minimum of 6 amino acids. However, longer polypeptides may advantageously bind to GDF15 with higher affinity, and thus a polypeptide may or comprise at least 7, 8, 9, 10, 11, 12, 13, 14, 15, 16, 17, 18 or 19 or more amino acids.

For example, with respect to polypeptides derived or obtained from ECD3 of QRFRPR (SEQ ID NO. 5), these may have or comprise 6 to 29 amino acids, e.g. 6 to 28 amino acids, e.g. any one of 6, 7, 8, 9 or 10, to any one of 27, 26, 25, 24, 23, 22, 21, 20, 19, or 18, for example amino acids from or corresponding to SEQ ID NO.5 or a part thereof. In certain embodiments, the polypeptide is or comprises no more than 27 amino acids of SEQ ID NO:5.

Surprisingly it was found that a short portion of the extracellular domain of ECD3 of QRFPR was capable of binding to GDF15 in solution (see Example 16). A polypeptide comprising 15 amino acids of ECD3 from mouse QRFPR was provided (SEQ ID NO:184) as an Fc fusion protein with a flexible linker and was found to bind well to GDF15 in a pull-down assay (see SEQ ID NO:240). This polypeptide corresponds to the sequence from human QRFPR having the sequence set forth in SEQ ID NO:176. The mouse sequence is highly homologous to the human sequence, and differs only in having an arginine residue at the second position of SEQ ID NO:184 (mouse), rather than a glutamine residue at the second position of SEQ ID NO:176 (human). Polypeptides having sequences derived from or closely related to this sequence are of particular interest. Polypeptides closely related to this sequence comprising 13-18 amino acids therefore represent particularly preferred embodiments of the present invention. In particular, polypeptides having or comprising a sequence as set forth in any one of SEQ ID NOs:30, 31, 32, 39, 46, 47 or 175-189 or a sequence having at least 75% identity thereto, represent preferred polypeptides of the various aspects of the present invention.

As noted above, such an amino acid sequence derived from ECD3 may be combined with a sequence from ECD4 of QRFPR. Thus, a polypeptide may comprise a first sequence being an aforesaid ECD3-derived sequence as defined in the paragraph above together with a second sequence as set forth in SEQ ID NO:12 or 13, or a sequence having a least 75% sequence identity thereto. The first and second sequences may be linked in either order, optionally via a linker or spacer sequence, or they may be provided in combination as a heterodimer, e.g. a Fc fusion protein heterodimer, as described in more detail below.

A polypeptide obtained or derived from ECD4 of QRFPR (SEQ ID NO. 12) may have or comprise 6 to 19 amino acids, e.g., 6 to 18, or any one of 6, 7, 8 or 9 to any one of 19, 18, 17, 16 or 15, for example amino acids from or corresponding to SEQ ID NO.12 or a part thereof. In certain embodiments, the polypeptide is or comprises no more than 17 amino acids of SEQ ID NO:12.

A polypeptide obtained or derived from the ECD of CLPTM1 (SEQ ID NO. 14) may comprise 6 to 354 amino acids, e.g. any one of 6, 7, 8, 9, 10, 12, 14, 16, 18 or 20 to any one of 354, 353, 350, 320, 300, 280, 260, 250, 240, 220, 200, 180, 160, 150, 140, 120, 100, 90, 80, 70, 60, 50, 40, 30 or 20, for example amino acids from or corresponding to SEQ ID NO.14 or a part thereof. For example, a polypeptide may have or comprise 6, 7, 8 or 9 to 50, 45, 40, 35, or 30 amino acids or more obtained or derived from SEQ ID NO. 14, or corresponding to a part thereof.

A polypeptide comprising a 31 amino acid portion of the extracellular domain of CLPTM1 corresponding to the sequence set forth in SEQ ID NO:17 was found to be capable of binding to GDF15 in solution (see Example 16) and blocking the effect of GDF15 on CLPTM1 activity in CD14+ immune cells (see Example 17). A fusion protein comprising the above polypeptide derived from CLPTM1 linked to Fc with a flexible linker comprising a triple repeat of the GGGGS linker sequence was provided (see SEQ ID NO:241). Polypeptides having or comprising the sequence of SEQ ID NO: 17, or a sequence having at least 75% identity thereto, or a polypeptide comprising a sequence which is a part of SEQ ID NO: 17 comprising at least 6 contiguous amino acids are therefore preferred polypeptides of the various aspects of the present invention.

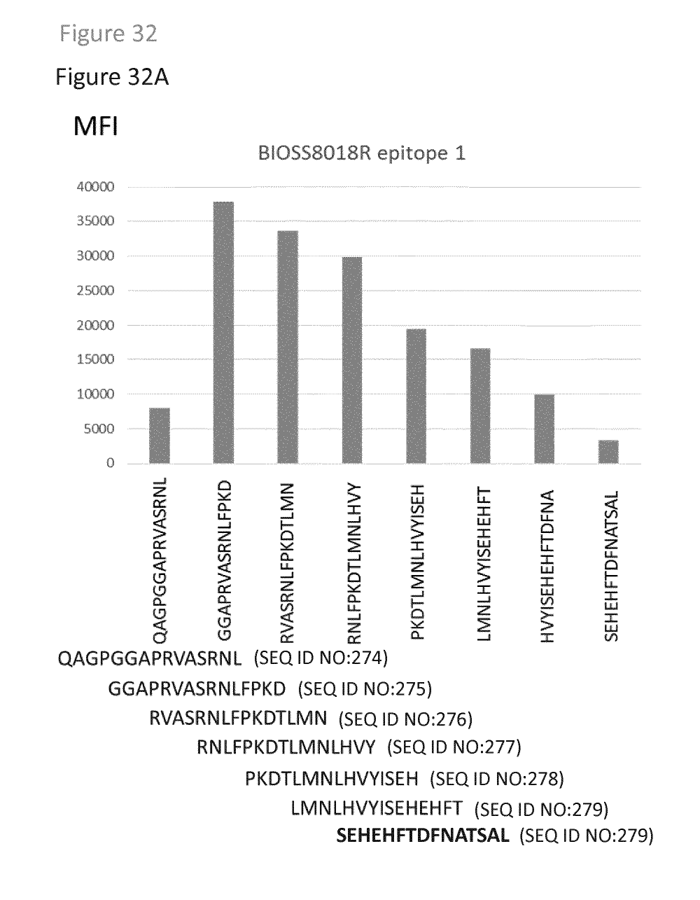

Binding of GDF15 to a number of polypeptides derived from CLPTM1 was tested in an ELISA assay in Example 18 (see FIG. 32). A number of polypeptides were found to bind to GDF15, and thus these may represent preferred polypeptides of the present invention. Thus, a polypeptide having or comprising a sequence as set forth in any one of SEQ ID NOs:258, 259, 261, 262, 269, 270, 297, 298, 301, 302, 308, 309, or a sequence having at least 75% identity thereto, represent preferred polypeptides of the various aspects of the present invention.

In particular, a polypeptide having or comprising, or based on or derived from (e.g. a part thereof comprising at least 6 contiguous amino acids or a sequence having at least 75% sequence identity to), the amino acid sequence set out in SEQ ID NO: 270 (namely the sequence GDYYPIIYFNDYWNLQKDYYG) or SEQ ID NO: 309 (namely the sequence GDYYPIIYFNDYWNLQKDYY, i.e. SEQ ID NO: 270 lacking the C-terminal G residue which is not present in the native ECD of CLPTM1) is preferred. For example the polypeptide may be in the form of a fusion protein comprising a polypeptide having the sequence of SEQ ID NO: 270 or 309 or a sequence which is a part of SEQ ID NO: 270 or 309 comprising at least 6 contiguous amino acids, or a sequence which has at least 75% identity to SEQ ID NO: 270 or 309. In another embodiment the polypeptide may not have a fusion partner, but may be a naked polypeptide, or the polypeptide may be conjugated to another moiety, e.g. a non-peptide based polymer such as is described further below. For example the polypeptide may comprise a part of SEQ ID NO:270 or 309 which comprises at least the sequence DYYPII (SEQ ID NO:326).

In certain embodiments of the present invention wherein the polypeptide is obtained or derived from the extracellular domain of CLPTM1, the polypeptide may comprise the native N-terminal methionine residue encoded by the CLPTM1 gene, e.g. where the native or endogenous CLPTM1 start codon is provided in a nucleic acid sequence encoding the polypeptide. The polypeptide may, therefore, comprise amino acids 1 to 137, 138, 139, 140, 150, 160, 180, 200, 220, 240, 260, 280, 288, 290, 300, 320, 340, 350 or 354 of the amino acid sequence set forth in SEQ ID NO:1. Thus, in a further embodiment the polypeptide may have or comprise an amino acid sequence corresponding to SEQ ID NO:14 together with an N-terminal methionine. Such a polypeptide is represented by SEQ ID NO:315. Accordingly, where SEQ ID NO.14 is referred to herein, for example in relation to polypeptides which comprise SEQ ID NO:14 or a part thereof, such a reference may be replaced or supplemented with a reference to SEQ ID NO. 315. In other words, any reference herein to SEQ ID NO. 14 may be taken also to include a reference to SEQ ID NO:315.

Indeed, a polypeptide of the invention may comprise or have a length within a range corresponding to between and including any integer included above, or lying within any of the ranges above. Furthermore, as will be described in more detail below, a polypeptide of the invention may be provided in fusion form or in other elongated or extended form, comprising one or more other amino acid sequences attached or coupled to the peptide sequence obtained or derived from QRFPR and/or CLPTM1.

In a particular embodiment of the present invention, the polypeptide may comprise all or substantially all of an entire extracellular domain of QRFPR and/or CLPTM1.

In order for the polypeptides to be able to block the interaction between GDF and its receptors, it is necessary that such polypeptides are capable of binding to GDF15. Without wishing to be bound by theory, it is believed that the binding sites on GDF15 for QRFPR and CLPTM1 are overlapping (either fully or partially) and thus a polypeptide derived from the extracellular domain of either of these receptors which binds to GDF15 is thought to be capable of blocking the binding of GDF15 to either receptor. In other words, a polypeptide obtained or derived from an extracellular domain of QRFPR that binds to GDF15 will be capable of preventing GDF15 binding to either QRFPR or CLPTM1, and a polypeptide obtained or derived from the extracellular domain of CLPTM1 that binds to GDF15 will be capable of preventing GDF15 binding to either QRFPR or CLPTM1.

This is supported by further work, reported in Example 20 below, which shows that there an area of sequence similarity between ECD3 of QRFPR and the ECD of CLPTM1. Some sequence similarity with ECD4 is also indicated. Interestingly, these are the only regions of similarity over background when the two receptors are compared and it is in a site, or area, mediating GDF15 binding.

A polypeptide of the invention may be provided in soluble form, e.g. as a soluble polypeptide, or a peptide which is provided with solubilising groups and/or moieties.

The polypeptide may be provided in monomeric, or in dimeric or higher multimeric form, i.e. it may be or more comprise a dimer or higher multimer of any of the polypeptides described or defined herein. Data provided in the Examples below shows that the polypeptides may have activity in either monomeric or dimeric form. GDF15 exists as a dimer, and hence there may be a benefit to providing the polypeptide in dimeric form.

Dimers or higher multimers may be formed in any convenient way, according to techniques known in the art. For example dimers may form spontaneously during the formation of fusion proteins, or they may be engineered.

It may advantageous or desirable to provide the polypeptide in the form of a fusion protein or construct or conjugate with a further moiety.

In certain embodiments, the polypeptide may be conjugated or linked to a natural or synthetic polymer, including a protein, polypeptide or peptide, or a polysaccharide, to improve its pharmacokinetic properties. In various embodiments the polypeptide may, therefore, be conjugated to linear or branched-chain monomethoxy poly-ethylene glycol (PEG; PEGylation), hyaluronic acid, dextran or dextrin, a homo-amino acid polymer (HAP; HAPylation), a proline-alanine-serine polymer (PAS; PASylation) or an elastin-like peptide (ELP; ELPylation) as described in Strohl. 2015. BioDrugs 29, 215-239, the entire disclosure of which is hereby incorporated by reference. Indeed, any known fusion or conjugation partner known in the art for use with therapeutic proteins may be used.

Any of the polypeptides of the present invention may be provided as a fusion protein or chimeric protein, for example in order to increase the serum half-life of the polypeptide and/or to impart a further functionality to the polypeptide. The term “fusion protein” refers to a single polypeptide chain comprising polypeptide sequences from two or more different sources. A polypeptide of the invention may be provided as a fusion protein in combination with an albumin, a fibrinogen, a glutathione S-transferase, a transferrin, streptavidin or a streptavidin-like protein, or an immunoglobulin, or a part thereof, in particular with the Fc portion of an immunoglobulin (e.g. IgG1, IgG2, IgG3 or IgG4), or a part thereof (e.g. as an Fc-fusion protein). Within a fusion protein comprising a polypeptide of the invention, the polypeptide of the invention may be situated at the N-terminus of the fusion protein, or at the C-terminus of the fusion protein. Alternatively, the polypeptide of the invention may be situated internally in the fusion protein, e.g. within a loop or other surface feature of the fusion protein.

In a preferred embodiment, a polypeptide of the invention may be provided as fusion protein in combination with the Fc region of an immunoglobulin, i.e. may be provided as an Fc-fusion protein. Fc-fusion proteins form dimers, and thus in this embodiment two copies of a poly peptide of the invention may be provided as a single entity. In such an embodiment the dimer may either be a homodimer, comprising two identical polypeptides of the invention, or may be provided as a heterodimer, comprising two non-identical polypeptides of the invention. For example, the Fc-fusion protein may be a homodimer comprising two identical polypeptides obtained or derived from the third extracellular domain of QRFPR, as described herein, or may be a homodimer comprising two identical polypeptides obtained or derived from the fourth extracellular domain of QRFPR as described herein. The Fc-fusion protein may also be a heterodimer comprising two different polypeptides obtained or derived from the third extracellular domain of QRFPR, as described herein, or may be a heterodimer comprising two different polypeptides obtained or derived from the fourth extracellular domain of QRFPR, as described herein. In a particular embodiment, the Fc-fusion protein may be a heterodimer comprising a first polypeptide obtained or derived from the third extracellular domain of QRFPR, and a second polypeptide obtained or derived from the fourth extracellular domain of QRFPR, as described herein. The Fc-fusion protein may also be a homodimer comprising two identical polypeptides derived from CLPTM1, as described herein, or may be a heterodimer comprising two different polypeptides derived from CLPTM1 as described herein. In a particular embodiment, the Fc-fusion protein may be a heterodimer comprising a first peptide comprising the sequence SEQ ID NO:15 or a sequence having at least 75% sequence identity thereto and a second peptide comprising the sequence SEQ ID NO:16 or a sequence having at least 75% sequence identity thereto.

As well as monomers and dimers, Fc fusions in higher multimeric forms, e.g. trimers or higher, are also known, and are included according to the present invention.

An Fc-fusion protein may comprise an Fc domain which is immunogenic. In other words, an Fc-fusion protein may comprise a sequence which is known to induce an immune response, e.g. a response by cells of the innate immune system. Thus, in one embodiment, an Fc-fusion protein may be immunogenic. In a particular embodiment, the immunogenic sequence may induce antibody-dependent cellular cytotoxicity (ADCC) and/or antibody dependent cell mediated phagocytosis (ADCP). Examples of immunogenic Fc-tag sequences are known in the art (see e.g. Lazar et al. 2006. PNAS 103, 4005-4010); Shields et al. 2001. J. Biol. Chem. 276, 6591-6604; and Stewart et al. 2011. Protein Engineering, Design and Selection 24, 671-678). Although not limited to such a use, such immunogenic Fc-fusion proteins may be of particular utility in the treatment of cancers which overexpress GDF15, and which have high concentrations of GDF15 proximal to their cell surface. Such Fc fusion proteins may act to recruit cells of a subject's immune system to a site of cancer, and induce an immune response in addition to simply blocking the interaction between GDF15 and its receptors.

It may be desirable, however, for an Fc-fusion protein to comprise minimal immunogenic activity, i.e. to bind to (and thus sequester) GDF15, without inducing an immune response. Thus, in a further embodiment, an Fc-fusion protein is not immunogenic.

In another preferred embodiment, a polypeptide may be provided as conjugate with a bisphosphonate. Without wishing to be bound by theory, it is believed that such a construct might be directed for enrichment (i.e. to accumulate) in bone. Such an accumulation may have particular advantages in preventing the interaction of GDF15 with its receptors in bone, which may be of particular benefit in inhibiting the engagement of bone in cancer.

In any of the fusion proteins or conjugates contemplated in the present invention, a polypeptide derived from QRFPR or CLPTM1 may be linked to is conjugate or fusion partner at its N-terminus and/or at its C-terminus by a linker sequence or linker or spacer group. For fusion proteins comprising a polypeptide/peptide based fusion partner the linker will conveniently be a peptide/polypeptide linker, many of which are known and described in the art. The linker may be a flexible linker sequence (which may include repeats of a flexible linker sequence motif). Typical linkers known in the art are rich in small non-polar (e.g. glycine) or polar (e.g. serine or threonine) residues, and commonly consist of stretches of glycine and serine residues (GS). A commonly used linker is the (GGGGS) linker (SEQ ID NO:242), which may be provided as a repeating unit in a linker (as (GGGGS)ₙ) where the copy number of n may be adjusted. Such a linker is used in the fusion protein constructs of Example 16 as a triple repeat (i.e. (GGGGS)₃).

For conjugates with non-polypeptide/peptide polymers (such as PEG or polysaccharides etc.) or with other conjugate partners, the polypeptide of the invention may be coupled or linked (conjugated) to the other moiety (conjugate partner) using any known or desired coupling chemistry or linker/coupling groups. A wide variety of these are described in the art.

In yet another embodiment, the polypeptides of the present invention may be provided as cyclic peptides. Cyclic peptides are of particular utility in the oral administration of a protein or peptide-based therapeutic agent, as they are typically highly resistant to proteolysis, and are thus not broken down by proteolytic enzymes in the digestive tract. However, cyclic peptides also typically exhibit increased serum half-life when compared with a structurally-related linear peptide.

Polypeptides are described herein which comprise one or more amino acid substitutions relative to the wild-type sequences for an extracellular domain of a GDF15 receptor, yet which retain the ability to bind to GDF15. A polypeptide of the present invention will preferably have a high degree of sequence identity to that of the equivalent section of the sequence found in the native protein. A polypeptide of the invention has at least 75% sequence identity and preferably will have at least 80%, 85%, 86%, 87%, 88%, 89%, 90%, 91%, 92%, 93%, 94%, 95%, 96%, 97%, 98% or 99% sequence identity to that of the equivalent section of the sequence found in the native protein. However, in a particular aspect of the present invention a polypeptide which is used to block the interaction of GDF15 and its receptors by binding to GDF15 may comprise an identical sequence to that of the equivalent section of the sequence found in the native protein. Without wishing to be bound by theory, it is possible that such a polypeptide may provoke less of an immune response (i.e. be less immunogenic) than a polypeptide having one or more substitutions relative to the native protein, as a polypeptide having a non-native sequence may be recognised as ‘foreign’ by a subject's immune system.

Sequence identity may readily be determined by methods and software known and readily available in the art. Thus, sequence identity may be assessed by any convenient method. However, for determining the degree of sequence identity between sequences, computer programs that make multiple alignments of sequences are useful, for instance Clustal W (Thompson et al., (1994) Nucleic Acids Res., 22: 4673-4680). Programs that compare and align pairs of sequences, like ALIGN (Myers et al., (1988) CABIOS, 4: 11-17), FASTA (Pearson et al., (1988) PNAS, 85:2444-2448; Pearson (1990), Methods Enzymol., 183: 63-98), BLAST and gapped BLAST (Altschul et al., (1997) Nucleic Acids Res., 25: 3389-3402) are also useful for this purpose, and may be used using default settings. Furthermore, the Dali server at the European Bioinformatics institute offers structure-based alignments of protein sequences (Holm (1993) J. Mol. Biol., 233: 123-38; Holm (1995) Trends Biochem. Sci., 20: 478-480; Holm (1998) Nucleic Acid Res., 26: 316-9). Multiple sequence alignments and percent identity calculations may be determined using the standard BLAST parameters, (using sequences from all organisms available, matrix Blosum 62, gap costs: existence 11, extension 1). Alternatively, the following program and parameters may be used: Program: Align Plus 4, version 4.10 (Sci Ed Central Clone Manager Professional Suite). DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix. Variants of the naturally occurring polypeptide sequences as defined herein can be generated synthetically e.g. by using standard molecular biology techniques that are known in the art, for example standard mutagenesis techniques such as site-directed or random mutagenesis (e.g. using gene shuffling or error prone PCR). Such mutagenesis techniques can be used to develop polypeptides which have improved or different binding and/or inhibitory properties.

Derivatives of the polypeptides as defined herein may also be used. By derivative is meant a polypeptide as described above or a variant thereof which instead of the naturally occurring amino acid contains a structural analogue of that amino acid. Derivatisation or modification (e.g. labelling, glycosylation, methylation of the amino acids in the polypeptide) may also occur as long as the function of the polypeptide is not adversely affected.

By “structural analogue”, it is meant a non-standard amino acid. Examples of such non-standard or structural analogue amino acids which may be used are D amino acids, amide isosteres (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acids, D-8 methylamino acids, or D-N-methylamino acids.

Where a polypeptide comprises an amino acid substitution relative to the sequence of the native protein, the substitution may preferably be a conservative substitution. The term “a conservative amino acid substitution” refers to any amino acid substitution in which an amino acid is replaced (substituted) with an amino acid having similar physicochemical properties, i.e. an amino acid of the same class/group. For instance, small residues Glycine (G), Alanine (A) Serine (S) or Threonine (T); hydrophobic or aliphatic residues Leucine (L), Isoleucine (I); Valine (V) or Methionine (M); hydrophilic residues Asparagine (N) and Glutamine (Q); acidic residues Aspartic acid (D) and Glutamic acid (E); positively-charged (basic) residues Arginine (R), Lysine (K) or Histidine (H); or aromatic residues Phenylalanine (F), Tyrosine (Y) and Tryptophan (W), may be substituted interchangeably without substantially altering the ability of a polypeptide to bind to GDF15.

A second class of agents comprises binding agents which are capable of binding to a GDF15 receptor and which may be used to block the interaction between GDF15 and its receptors. Such binding agents may be used to reduce the interaction between GDF15 and its receptors by binding to the extracellular domain of a receptor and preventing GDF15 from binding to said receptor, or by inhibiting or reducing the effect of GDF15 binding at the receptor (as noted above, this may not necessarily involve prevention of GDF15 binding).

Since such binding agents act to inhibit, or reduce, the binding, or effect or activity of GDF15 at the receptor they may be viewed as receptor antagonists, at least with respect to the effect of the ligand GDF15 at the receptor. Such antagonists included partial antagonists (that is the binding agent may have a desired antagonistic effect with respect to GDF15, but may also exhibit an agonistic activity at the receptor with respect to other effects/other ligands at the receptor).

In one embodiment, the agent may bind to an extracellular domain of QRFPR and reduce the ability of QRFPR to bind to GDF15, and/or the ability of GDF15 to exert its effect at the receptor. QRFPR comprises four extracellular domains as discussed above. A binding agent may block the interaction between GDF15 and QRFPR by binding to any of these domains in a manner which prevents the binding of GDF15, either by binding directly to the GDF15 binding site (and thus blocking GDF15 from binding to QRFPR), or by binding in sufficient proximity to the GDF15 binding site that it sterically prevents or hinders GDF15 from binding to the receptor.

In a particular embodiment, the binding agent may bind to the same regions of QRFPR which have been found to interact with GDF15 (the ligand binding site), i.e. to one or both of the third and fourth extracellular domains of QRFPR. Thus in one embodiment, the agent may bind to the third extracellular domain of QRFPR (i.e. to a polypeptide having the amino acid sequence as set forth in SEQ ID NO:5), or the fourth extracellular domain of QRFPR (i.e. to a polypeptide having the amino acid sequence as set forth in SEQ ID NO:12), or to both the third and fourth extracellular domains of QRFPR (i.e. to a polypeptide having the amino acid sequence as set forth in SEQ ID NO:5 and to a polypeptide having the amino acid sequence as set forth in SEQ ID NO:6, or more particularly to a polypeptide comprising both SEQ ID NO. 5 and 12. In a further embodiment, the agent may only bind to a portion of the third and/or fourth extracellular domains of QRFPR.

In a further embodiment, the binding agent may not bind to the GDF15 binding site. In this embodiment the agent is capable of blocking the interaction between GDF15 and QRFPR by sterically hindering the interaction. Thus, the binding agent may prevent the binding of GDF15 to QRFPR by binding to the first and/or second extracellular domains of QRFPR. In one embodiment, the agent may bind to the first extracellular domain of QRFPR (i.e. to a polypeptide having the amino acid sequence as set forth in SEQ ID NO:3). In a further embodiment, the binding agent may bind to the second extracellular domain of QRFPR (i.e. to a polypeptide having the amino acid sequence as set forth in SEQ ID NO:4). In yet another embodiment, the binding agent may bind to both the first and second extracellular domains of QRFPR (i.e. to a polypeptide having the amino acid sequence as set forth in SEQ ID NO:3 and to a polypeptide having the amino acid sequence as set forth in SEQ ID NO:4 or to polypeptide comprising both SEQ ID NO. 3 and 4).

In an alternative embodiment, the agent may bind to the extracellular domain of CLPTM1, and reduce the ability of CLPTM1 to bind to GDF15, and/or the ability of GDF15 to exert its effect at the receptor. Thus, in a first embodiment the agent may bind to a polypeptide having an amino acid sequence as set forth in SEQ ID NO:14. In a particular embodiment, the agent may bind to a polypeptide having or comprising an amino acid sequence as set forth in SEQ ID NO:17. In a further embodiment, the agent may bind to a polypeptide having or comprising an amino acid sequence as set forth in SEQ ID NO:18. In yet another embodiment, the agent may bind to a polypeptide having or comprising an amino acid sequence as set forth in SEQ ID NO:15 and/or to a polypeptide having or comprising an amino acid sequence as set forth in SEQ ID NO:16. In another embodiment, the agent may bind to a polypeptide having or comprising an amino acid sequence as set forth in SEQ ID NO:200.

Example 19 below describes the elucidation of the binding site in the ECD of CLPTM1 (SEQ ID NO:14) for an anti-CLPTM1 antibody which demonstrates antagonist activity against this receptor. The common denominator of this antibody is indicated to be PKD (SEQ ID NO: 322) at positions 98-100 of SEQ ID NO:2. Accordingly, in a still further embodiment, the agent may bind to a polypeptide having or comprising the sequence PKD (SEQ ID NO:322), e.g. a polypeptide having or comprising an amino acid sequence as set forth in SEQ ID NO:212 (PKDT), or having or comprising any one of SEQ ID NOs:213-239 or any one of SEQ ID NOs: 275 to 278. A second epitope is also identified in Example 19. Accordingly, in a yet further embodiment the agent may bind to a polypeptide having or comprising an amino acid sequence as set forth in any one of SEQ ID NOs:280-286

A binding agent of the invention may be any agent, e.g. any compound, molecule or entity having the ability to bind to QRFPR, and particularly to an extracellular domain of QRFPR and/or CLPTM1. In particular the binding agent may bind specifically to QRFPR and/or CLPTM1 (or an extracellular domain thereof). By binding specifically is meant that the agent is capable of binding to QRFPR and/or CLPTM1 (or an extracellular domain thereof) in a manner which distinguishes it from binding to a non-target molecule. Thus, binding to a non-target molecule may be negligible or substantially reduced as compared to binding to QRFPR and/or CLPTM1. A binding agent may thus be any agent having a binding affinity for QRFPR and/or CLPTM1 i.e. an affinity binding partner for QRFPR and/or CLPTM1, or more particularly for an extracellular domain thereof.

Binding agents of the present invention may thus be selected from proteins or polypeptides such as antibodies or fragments or derivatives thereof, a combinatorially derived polypeptide from phage display or ribosome display or any other peptide display system, non-agonistic fragments of GDF15 (e.g. polypeptides comprising a portion of GDF15), or a nucleic acid molecule, such as an aptamer, or combinations thereof.

In a preferred embodiment of the invention, the binding agent is a protein, preferably an antibody or derivative or fragment thereof. Various antibody-like molecules are also known and described in the art and may be used, e.g. affibodies and such like.

In a preferred embodiment, the binding agent is an antibody. The antibody may be of any convenient or desired species, class or sub-type. Furthermore, the antibody may be natural, derivatised or synthetic. The term “antibody” as used herein thus includes all types of antibody molecules and antibody fragments.

More particularly the “antibody” according to the present invention includes:

(a) any of the various classes or subclasses of immunoglobulin e.g. IgG, IgA, IgM, IgD or IgE derived from any animal e.g. any of the animals conventionally used e.g. sheep, rabbits, goats, or mice or egg yolk;

(b) monoclonal or polyclonal antibodies;

(c) intact antibodies or fragments of antibodies, monoclonal or polyclonal, the fragments being those which contain the binding region of the antibody e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains;

(d) antibodies produced or modified by recombinant DNA or other synthetic techniques, including monoclonal antibodies, fragments of antibodies, humanised antibodies, chimeric antibodies, or synthetically made or altered antibody-like structures. Also included are functional derivatives or “equivalents” of antibodies e.g. single chain antibodies.

A single chain antibody may be defined as a genetically engineered molecule containing the variable region of the light chain, the variable region of the heavy chain, linked by a suitable polypeptide linker as a fused single chain molecule.

Methods of making such antibody fragments and synthetic and derivatised antibodies are well known in the art. Also included are antibody fragments containing the complementarity-determining regions (CDRs) or hypervariable regions of the antibodies. These may be defined as the region comprising the amino acid sequences on the light and heavy chains of an antibody which form the three dimensional loop structure that contributes to the formation of the antigen binding site. CDRs may be used to generate CDR-grafted antibodies. As used herein “CDR grafted” defines an antibody having an amino acid sequence in which at least parts of one or more sequences in the light and/or variable domains have been replaced by analogous parts of CDR sequences from an antibody having a different binding specificity for a given antigen. One of skill in the art can readily produce such CDR grafted antibodies using methods well known in the art.

A chimeric antibody may be prepared by combining the variable domain of an anti-receptor antibody of one species with the constant regions of an antibody derived from a different species. Such techniques may be used to humanise antibodies for therapeutic use.

Monoclonal antibodies and their fragments and derivatives are preferred antibodies according to the present invention.

Preferably the antibody will be a humanised or chimeric antibody, i.e. an antibody which has been modified or created to comprise a binding domain (e.g. a complementarity determining region (CDR)) which recognises a human GDF15 receptor and a fixed (e.g. Fc) domain that has been modified to increase their similarity to antibody variants produced naturally in humans. In a particular aspect, a humanised or chimeric monoclonal antibody may be the IgG4 subtype. Modifications of this type are desirable to provide antibodies which have a minimal immunogenic effect.

In another preferred embodiment the antibody may be a human antibody. Human antibodies may be prepared using transgenic mice or other transgenic animals which may have been modified to express human immunoglobulin genes. They may also be obtained from phage display or indeed they may be isolated from human subjects, namely a human subject in whom the anti-receptor antibodies natively exist or are present (i.e. without the need to immunise the subject for antibody production), for example a human auto-immune subject.

Antibodies binding to QRFPR or CLPTM1 have been reported and are commercially available (see for example in the Examples below). One such example is the anti-CLPTM1 antibody bs-8018R available from Bioss Antibodies (USA). Furthermore, antibodies to the receptors may readily be prepared using known and routine techniques. Methods for determining the binding site of an antibody in its antigen (the moiety to which it binds) are also routine (see also the Examples below) and so it is readily possible to determine the epitope, or antibody binding site in QRFPR or CLPTM1.

As noted above the polypeptides and binding agents of the invention may be provided in the form of compositions, and in particular pharmaceutical compositions which may be used, or which may be provided for use, in treating or preventing a condition associated with elevated GDF15. Such compositions may comprise or contain one or more polypeptides and/or one or more binding agents of the invention. The polypeptides and binding agents may be provided together in a single composition or in separate compositions for use together or in conjunction (i.e. as a combination therapy). The polypeptide and binding agent may be administered or used sequentially or simultaneously or substantially simultaneously. Thus the different polypeptide and binding agent components may be administered or used, together or separately, at substantially the same time, or separately, each administered or used separately at different times or time intervals.

Such a composition or combination (e.g. kit or combined product as described above) may comprise a polypeptide obtained or derived from an extracellular domain of QRFRP and a binding agent which binds to an extracellular domain of QRFPR. In a further embodiment, a composition or combination may comprise a polypeptide obtained or derived from an extracellular domain of QRFPR and a binding agent which binds to the extracellular domain of CLPTM1. In another embodiment, a composition or combination may comprise a polypeptide obtained or derived from the extracellular domain of CLPTM1 and a binding agent which binds to the extracellular domain of QRFPR. In a still further embodiment, a composition or combination may comprise a polypeptide obtained or derived from the extracellular domain of CLPTM1 and a binding agent which binds to the extracellular domain of CLPTM1.

It will be apparent that a composition, kit or combined product of the present invention may comprise a polypeptide obtained or derived from a GDF15 receptor and a binding agent which binds to the same receptor. In this case, it is generally desirable that the binding agent does not bind to the polypeptide derived from the same receptor, as the binding agent and polypeptide would bind to each other and thus not be able to block the interaction with GDF15 with its receptors. This may be achieved in various ways. In one embodiment, the binding agent may bind to a portion of the GDF15 receptor which is not directly involved in binding GDF15 (and thus may block GDF15 binding indirectly through steric hindrance as discussed above), and the polypeptide may be derived from a portion of the GDF15 receptor which binds to GDF15. In a further embodiment, the binding agent may bind to a portion of the GDF15 receptor which is involved in binding GDF15, and the polypeptide may be derived from a different portion of the GDF15 receptor. By way of example, the binding agent may bind to the fourth extracellular domain of QRFPR, and the polypeptide may be derived from the third extracellular domain of QRFPR. In yet another embodiment, the polypeptide may comprise a sequence modification, e.g. a conservative substitution, which does not impair its ability to bind to GDF15, but which prevents it from binding to the binding agent. However, the skilled person will appreciate that it would be straightforward to establish whether a particular binding agent might bind to a particular polypeptide, and that such combinations may be avoided.

Thus, by way of example, an antibody may be used which demonstrates an epitope shift with respect to the binding site in the receptor for GDF15, allowing such an antibody to be used in the same subject together with a polypeptide obtained or derived from the same receptor, without interfering with one another.

The advantage of a binding agent which does not bind to a polypeptide of the invention, or at least does not bind to a polypeptide with which it is used in conjunction, is that both components can be used together, and may advantageously be used to obtain a complementary effect, binding both to GDF15, e.g. GDF15 which is free in the circulation, or located in the site of vicinity of a tumour, and to receptors to which the GDF15 may bind to exert deleterious or unwanted effects.

As noted previously, several diseases, disorders and conditions are associated with increased levels of GDF15. Such increased levels of GDF15 may be detectable in the circulation or in tissues or organs of the body. GDF15 may also increase locally at the site or vicinity of a tumour, for example it may accumulate in or around the stroma or extracellular matrix of a tumour.

A condition associated with elevated levels of GDF15 may be any condition, disease or disorder in which the level of GDF15 is increased, including in the circulation and/or locally, e.g. at a site of, or in the vicinity of, a cancer (for example a tumour) or other injury or disorder (e.g. at a site of inflammation). For instance, where GDF15 is increased locally at the site of a cancer (e.g. tumour), the increase may not be such as to have an appreciable effect on circulating levels of GDF15, but the local concentration or amount of GDF15 may be increased (for example in the microenvironment of the tumour). Thus the level of GDF15 may be increased relative to a subject without the condition in question e.g. a healthy subject, or relative to site or location in the body of the subject in which the condition is not, or was not, present.

A condition associated with elevated levels of GDF15 may thus include any condition in which the level of GDF15 is aberrant, whether in the circulation or locally at any site in the body. Also included according to the present invention is any condition in which the level of GDF15 is unwanted, e.g. results in a deleterious or harmful effect on the subject. Further included is any condition in which activity of GDF15 is increased or unwanted.

Such a condition may include any of the conditions mentioned above which are known to be associated with increased or elevated GDF15 levels. Accordingly, the condition may particularly include cachexia, including cachexia associated with or arising from any cause. Cachexia can arise from any chronic or long-term condition, including an inflammatory condition such as congestive heart failure, chronic obstructive pulmonary disease (COPD), chronic obstructive pulmonary hypertension, chronic kidney disease, cystic fibrosis, multiple sclerosis, motor neurone disease, Parkinson's disease, dementia, HIV/AIDS, rheumatoid arthritis, or any other inflammatory or autoimmune condition, including e.g. lupus, multiple sclerosis or ankylosing spondylitis.

In a particular embodiment the cachexia is cancer-induced.

More generally the condition may be unwanted or excessive weight loss, or any condition associated with or arising from appetite suppression and/or weight loss. The weight loss may be such that it is harmful to the patient, i.e. clinically significant weight loss. Examples include anorexia Cachexia, weight loss or appetite suppression may be associated with elevated circulating levels of GDF15. With respect to cachexia, weight loss or appetite suppression, the binding agent may particularly be a binding agent against the receptor QRFPR. The polypeptide may be any polypeptide of the invention, but may particularly be a polypeptide derived or obtained from QRFPR.

Further conditions include cancer, bone disorders, cardiovascular disease, lung disorders including for example chronic pulmonary disorders, e.g. chronic obstructive pulmonary disease (COPD), pulmonary arterial hypertension and renal disorders or diseases.

A number of blood and iron overload disorders have also been associated with elevated GDF15, and thus such a condition may also include Sickle Cell Disease, Hereditary Spherocytosis, Congenital Dyserythropoietic Anaemia types I and II, Thalassemia, Refractory anaemia with ring-sideroblasts (RARS), and Pyruvate kinase deficiency.

Cancer represents a condition of particular interest according to the present invention. Elevated GDF15 levels, whether circulating or locally at the site of a cancer, are known to indicate a poor prognosis for a wide range of different cancers, and many cancers are known to be overexpress and secrete GDF15. For example, prostate cancer (Karan et al. Cancer Res 2009; 69(1):2-5), multiple myeloma (Tanno et al. Blood. 2014 Jan. 30; 123(5):725-33), melanoma (Boyle et al. J Invest Dermatol. 2009 February; 129(2):383-91), colorectal cancer (Barderas et al. Mol Cell Proteomics. 2013 June; 12(6):1602-20), gastric cancer (Baek et al. Clin Chim Acta. 2009 March; 401(1-2):128-33), breast cancer (Kim et al. Carcinogenesis. 2008 April; 29(4):704-12), ovarian cancer (Chudecka-Glaz et al. Onco Targets Ther. 2015 Feb. 23; 8:471-85), endometrial cancer (Alonso-Alconada et al. Mol Cancer. 2014; 13: 223), oral squamous carcinoma (Urakawa et al. Lab Invest. 2015 May; 95(5):491-503), pancreatic cancer (Wang et al. BMC Cancer. 2014 Aug. 8; 14:578), lung cancer (Kadara et al. Cancer Biol Ther. 2006 May; 5(5):518-22), oral cancer (Schiegnitz et al. J Oral Pathol Med. 2015 Apr. 16), oesophageal cancer (Fisher et al. Br J Cancer. 2015 Apr. 14; 112(8):1384-91), testicular cancer (Altena et al. PLoS One. 2015 Jan. 15; 10(1):e0115372) and liver cancer (Si et al. PLoS One. 2011; 6(5):e19967) are associated with elevated GDF15 levels. Agents which are capable of reducing the activity of GDF15 to bind to its receptors would thus represent an attractive possible therapy in the field of oncology.

It is thought that in particular the interaction between GDF15 and CLPTM1 may be of particular significance in the pathology of many different cancers, in particular with regard to the modulation of immune function that is associated with elevated levels of GDF15. However, in certain cancers, in particularly bone cancers or metastases of other cancers to bone, the interaction between GDF15 and QRFPR may be of significance.

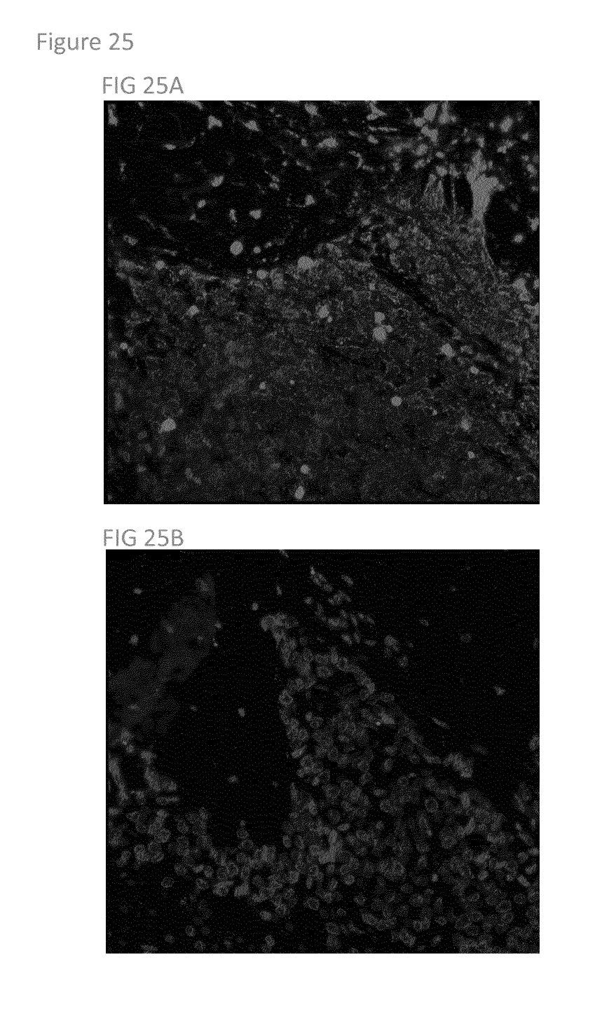

The term “cancer” is used broadly herein to include any malignant, non-malignant or pre-malignant neoplasm, including both solid and non-solid tumours, e.g. haemopoietic cancers. Any known cancer, including a cancer of any tissue or cell of the body is envisaged, and different cancer types, including but not limited to those listed above. In certain embodiments, however, the cancer is not rectal, colon, or prostate cancer (or is not prostate or rectal cancer). In another embodiment it is not any one or more of breast, cervical, or endometrial cancer, or it is not a glioma, a sarcoma other than Ewing Sarcoma, mesothelioma or blood cell cancer. In other embodiments such cancers are included. Our data appears to show that Ewing sarcoma is associated with high levels of GDF15 (see FIG. 25). Any polypeptide and/or binding agent of the invention may be used, including any of the combinations discussed above. Thus the polypeptide may be derived or obtained from QRFPR and/or CLPTM1, and any such polypeptide may be used in conjunction with any binding agent, that is a binding agent against QRFPR and/or CLPTM1, However, in certain preferred embodiments the binding agent is capable of binding to CLPTM1.

In a particular embodiment, the polypeptide may be obtained or derived from QRFPR and may be used in conjunction with a binding agent against CLPTM1. Alternatively, a polypeptide obtained or derived from CLPTM1 may be used in conjunction with a binding agent against CLPTM1.

The cancer may be a primary or secondary cancer, i.e. a cancer which has metastasised to a secondary site in the body, including micrometastases.

The cancer may be prostate cancer, bladder cancer, multiple myeloma, melanoma, colorectal cancer (including colon cancer and rectal cancer), kidney cancer, gastric cancer, breast cancer, ovarian cancer, endometrial cancer, oral squamous carcinoma, pancreatic cancer, lung cancer, oral cancer, oesophageal cancer, testicular cancer or liver cancer.

As noted above, we have particularly shown that that QRFPR may be expressed in various cancers of, or involving, the bone, including both primary cancers and metastases, particularly metastasis of prostate cancer to the bone. Thus, the cancer may particularly be a bone cancer or a cancer which involves the bone, e.g. Ewing Sarcoma, osteosarcoma, or multiple myeloma, or secondary prostate or other cancer metastases to bone

GDF15 is capable of down-regulating the immune response provided by a number of different immune cells, for example Natural Killer cells (NK-cells), macrophages and/or dendritic cells. As noted above, CLPTM1 has been shown to be expressed by various immune cells, including CD4 and CD8 positive T-lymphocytes, CD14 positive monocytes/macrophages, CD11c positive dendritic cells and/or NK cells. In work underlying the present invention we have thus shown that T-cell and/or other lymphocyte sub-sets may express CLPTM1.

Interestingly, the majority of CD14 positive cells (monocytes/macrophages) were markedly positive for CLPTM1. GDF15 has previously been reported to inhibit macrophage activation, and is also known as macrophage inhibitory cytokine 1 (MIC1). Stimulation of CLPTM1 by GDF15 results in increased phosphorylation of GSK3B and reduced immune function. GDF15 stimulation of CLPTM1 has also been found in the work leading up to the present invention to reduce the levels of NKG2D, and it was found that blocking the accessibility of GDF15 to CLPTM1 using a CLPTM1 binding agent (an antibody) reduced the extent to which the levels of NKG2D were reduced. NKG2D is expressed in both NK-cells and T-cells. NKG2D is a key effector protein for cytolytic function, such as Perforin and Granzyme B release. As GDF15 has been shown to reduce NKG2D levels in cytotoxic cells (thus disrupting cytolytic functions such as Perforin and Granzyme), we foresee that CLPTM1 binding agents may be of particular utility in immune-oncology applications where tumour derived GDF15 reduces the capacity of immune effector cells such as NKG2D positive NK and T-cells to mediate a cytolytic action. Additionally, we foresee that the inhibition of GDF15-induced proximity of CLPTM1 to the TGFb receptor complex (TGFB1(ALK5)/TGFBRII) may be of benefit in order to block downstream signalling through this complex that is inhibitory to immune cells.

Elevated GDF15 levels in the tumour stroma or at sites of metastasis (including micrometastasis) are thought to provide a significant mechanism for evasion of the immune system by cancer cells. The binding agents and/or polypeptides of the present invention may therefore have utility in preventing or reducing the immunosuppressive effects of GDF15.

Thus, more generally the therapeutic agents of the invention may be used to inhibit immunosuppression caused by or resulting from GDF15, or immunosuppression associated with an elevated level of GDF15.

Such immunosuppression may in particular be suppression of a cell-mediated cytotoxic immune response. Thus the immunosuppression may be immunosuppression resulting from decreased activity of one or more immune cells, e.g. macrophages, NK cells, dendritic cells, neutrophils, and/or T-cells.

More specifically the immunosuppression may be in the context of cancer i.e. immunosuppression associated with cancer. The immunosuppression may be due to increased circulating levels of GDF15, or in certain preferred embodiments, due to an elevated local concentration of GDF15 at the site of the cancer, e.g. tumour.

In particular, the binding agents and polypeptides of the present invention may be of utility in inhibiting immune evasion by cancer cells, or inhibiting immune tolerance induced by cancer cells. Therapeutic agents of the invention which block the interaction between GDF15 and QRFPR or CLPTM1, particularly CLPTM1 may be of utility in inhibiting immune evasion by cancer cells. Thus, peptides obtained or derived from CLPTM1 and/or QRFPR, and binding agents which are capable of binding to CLPTM1 and/or QRFPR, particularly CLPTM1 may of particular utility in this regard. As discussed any of the combinations of therapeutic agent discussed herein may be used.

By inhibiting immune evasion, the therapeutic agents of the invention may help the body of the subject to attack the cancer. Thus, the invention may assist in reducing or abrogating a cancer in the body of a subject by promoting (or facilitating or increasing or in any way enabling or assisting) an immune response in the body against the cancer.

In one particular aspect the therapeutic agents may be used to inhibit spread or metastasis of the cancer. This may include inhibiting the development of micrometastasis, or inhibiting the growth or spread of a micrometastasis. The metastasis may be of any cancer to any part of the body. In particular embodiments it may a metastasis of prostate, breast or lung cancer, including for example to the bone.

In the treatment of cancer the therapeutic agents of the invention may be used in combination or conjunction with other anti-cancer therapies, e.g. with other anti-cancer agents, including other immunotherapeutic or immunoncological agents or chemotherapeutic agents. Thus, for example, the agents of the invention may be used in conjunction with cell-based cancer therapies, e.g. adoptive cell transfer therapy and/or with antibody-based therapies.

In a particular embodiment, the agents of the present invention may be used in conjunction with adoptive cell transfer therapy, particularly NK-cells or T-cells, which may be in some embodiments be modified to express a chimeric antigen receptor (CAR), or T-cells expressing or modified to express (i.e. comprising a native or a heterologous) T-cell receptor, which has specificity for an antigen present on the surface of a cancer cell.

The present invention also provides cytotoxic immune cells which are modified to have a reduced level and/or activity of CLPTM1 compared with a cell which has not been modified, e.g. by gene knockout, gene knockdown or gene deletion.

Thus, the gene (i.e. the sequence in the cell encoding the CLPTM1 protein) may be modified to reduce the amount of the CLPTM1 protein expressed, for example the amount of CLPTM1 receptor at the cell surface, and/or the activity of the receptor protein. Thus, the receptor protein may be inactivated. This may involve, for example, modifying the gene sequence by and/or insertion or replacement/substitution of the native nucleotide sequence e.g. of a nucleotide sequence of the native CLPTM1 gene. This may be achieved by standard mutagenesis techniques and/or homologous substitution as known in the art.

In particular embodiments, the cell may be a T-cell expressing or modified to express a T-cell receptor or a T-cell modified to express a CAR. In a further embodiment, the cell may be an NK cell, optionally modified to express a CAR. Said modified cells may be or particular utility in the treatment of cancer, and thus in one embodiment, the cells will comprise a TCR or a CAR having specificity towards an antigen on the surface of a cancer cell. Use of said cells in therapy, particularly cancer therapy is also provided, as well as a combined product or preparation (e.g. a kit) comprising said cells and a therapeutic agent, being a polypeptide and/or binding agent of the present invention, for separate, sequential or simultaneous use in treating cancer. Thus a method, or use, of the present invention to treat cancer may comprise administering to a subject (in particular a subject in need thereof) a polypeptide and/or binding agent of the invention and (i.e. together, or in combination or conjunction, with) a said modified cytotoxic immune cell. The cell and therapeutic agent (polypeptide and/or binding agent) may be administered separately, sequentially or simultaneously, e.g. in separate formulations, as discussed above. Accordingly, a kit of the invention may comprise a polypeptide and/or a binding agent, and a modified cell, as hereinbefore described.

The agents of the present invention may also be used in conjunction with immunotherapeutic agents that target an immune checkpoint, i.e. immune checkpoint inhibitors in the treatment of cancer. Checkpoint proteins keep the immune system in check by indicating to the immune system which cells are healthy and which cells should be destroyed. Checkpoint proteins act as a “brake” on the immune system by preventing T-cell activation. If a cell does not have sufficient checkpoint proteins on its surface it may be destroyed by the immune system. In the case of cancer cells, whilst there may be molecules signalling that the cell is cancerous, if there are enough checkpoint proteins on the cell surface, the cell may evade the immune response, and it has been speculated that checkpoint proteins contribute to a lack of success in some cancer immunotherapies.

The best known example of a checkpoint protein is PD-L1 (for Programmed Death Ligand 1). The receptor for PD-L1 is PD-1. PD-L1 prevents T-cells from attacking healthy cells. Cancer cells may upregulate PD-L1 as a protective mechanism. When PD-L1 activates the PD-1 receptor on the surface of a T-cell, the T-cell is signalled to destroy itself. If the T-cells are programmed to selectively attack cancer cells, that set of T-cells will be destroyed and the cancer prevails.

Another checkpoint protein is cytotoxic T-lymphocyte antigen-4, or CTLA4. Once a cytotoxic T cell becomes active it expresses CTLA4 on its surface, which then competes with the co-stimulatory molecule CD28 for their mutually shared ligands, B7-1 and B7-2 on antigen-presenting cells. This balance holds cytotoxic activity in check, while allowing T cell function to proceed in a self-limited manner.

Other checkpoint proteins include CD-137 (4-1BB) which is a costimulatory checkpoint protein; lymphocyte activation gene 3 (LAG-3, CD223), a CD4-related inhibitory receptor coexpressed with PD-1 on tolerant T cells; B7 superfamily proteins B7-H3 and B7-H4; T cell protein TIM3; and phosphatidylserine (PS) which is a phospholipid in normal cells that is translocated to the outer member surface during apoptosis, suppressing the excess immune activation that would otherwise occur during processing and clearance of decaying cell matter. Externalization of PS indirectly stimulates macrophages, resulting in suppression of dendritic ell antigen presentation. Like PD-L1, externalized PS is aberrantly expressed by some tumour cells and tumour-derived microvesicles. Thus, PS is believed to be exploited by tumours to prevent adaptive tumour immunity.

Immunotherapeutic agents which may target or inhibit any of these checkpoint proteins are known as “checkpoint inhibitors”.

Checkpoint inhibitors (also known as immune checkpoint modulators, or CPMs) are designed to lessen the effectiveness of checkpoint proteins. Ideally a CPM should expose cancers to the immune system without causing that same system to attack healthy tissue.

Several checkpoint inhibitors are known and can be used in conjunction with the agents of present invention in the treatment of cancer, for example those inhibitors described in Creelan (2014) Cancer Control 21:80-89, which is hereby incorporated by reference.

Examples of checkpoint inhibitors include: Tremelimumab (CP-675,206), a human IgG2 monoclonal antibody with high affinity to CTLA-4; Ipilimumab (MDX-010), a human IgG1 monoclonal antibody to CTLA-4; Nivolumab (BMS-936558), a human monoclonal anti-PD1 IgG4 antibody that essentially lacks detectable antibody-dependent cellular cytotoxicity (ADCC); MK-3475 (formerly lambrolizumab), a humanized IgG4 anti-PD-1 antibody that contains a mutation at C228P designed to prevent Fc-mediated ADCC; Urelumab (BMS-663513), a fully human IgG4 monoclonal anti-CD137 antibody; anti-LAG-3 monoclonal antibody (BMS-986016); and Bavituximab (chimeric 3G4), a chimeric IgG3 antibody against PS. All of these checkpoint inhibitors can be used in the present invention.

An alternative strategy is to inhibit PD-L1, the ligand for PD-1, on the tumour cell surface, and therefore inhibitors of PD-L1 are may also be used in conjunction with the agents of the present invention, for example, MPDL3280A (RG7446), a human IgG1-kappa anti-PD-L1 monoclonal antibody. MED14736 is another IgG1-kappa PD-L1 inhibitor.

Another alternative approach is to competitively block the PD-1 receptor, using a B7-DC-Fc fusion protein, and such fusion proteins can also therefore be used in the present invention.

In a further alternative approach an antibody to Killer cell immunoglobulin-like receptor may be used as the immunotherapeutic agent. Killer cell immunoglobulin-like receptor (KIR) is a receptor on NK cells that downregulates NK cytotoxic activity. HLA class I allele-specific KIR receptors are expressed in cytolytic (CD56dimCD16+) NK cells, while CD56brightCD16− NK subset lacks these KIRs. Along these lines, inhibitory KIRs seem to be selectively expressed in the peritumoral NK cell infiltrate and thus seem to be a checkpoint pathway co-opted by tumours, similar to PD-L1. As such, inhibition of specific KIRs using antibodies should cause sustained in vivo activation of NK cells. For example, lirilumab (IPH2102) is fully human monoclonal antibody to KIR and can be used according to the invention.

Any suitable antibody which recognises and binds to a cancer antigen may also be used in conjunction with the agents of the invention. Examples of cancer antigens, or targets for therapeutic antibodies, include many “CD” proteins, such as CD52, CD47, CD30, CD33, CD20, CD152 and CD279; growth factors such as vascular endothelial growth factor (VEGF); growth factor receptors such as epidermal growth factor receptor (EGFR) or human epidermal growth factor receptor 2 (HER2).

Several antibodies that bind to such antigens or targets are known and have been approved for the treatment of cancer, and any of these antibodies may be used conjunction with the agents of the present invention. Preferred antibodies are those that have utility in treating solid tumours, and especially those with an altered ECM, such as breast, ovarian and pancreatic cancers.

Known and approved antibodies include: Alemtuzumab, Bevacizumab, Brentuximab vedotin, Cetuximab, Gemtuzumab ozogamicin, Herceptin, Ibritumomab tiuxetan, Ipilimumab, Ofatumumab, Panitumumab, Rituximab, Tositumomab and Trastuzumab.

Alemtuzumab is an anti-CD52 humanized IgG1 monoclonal antibody indicated for the treatment of fludarabine-refractory chronic lymphocytic leukemia (CLL), cutaneous T-cell lymphoma, peripheral T-cell lymphoma and T-cell prolymphocytic leukemia.

Bevacizumab (Avastin) is a humanized IgG1 monoclonal antibody which binds to vascular endothelial growth factor-A (VEGF-A) (referred to commonly as VEGF without a suffix). Bevacizumab binds to and physically blocks VEGF, preventing receptor activation which has consequences for tumour vascularisation. Bevacizumab is licensed for colon cancer, kidney cancer, lung cancer, ovarian cancer, glioblastoma and breast cancer.

Brentuximab vedotin is a second generation chimeric IgG1 antibody drug conjugate used in the treatment of Hodgkin lymphoma and anaplastic large cell lymphoma (ALCL). It is an antibody conjugated to monomethyl auristatin E, a drug that prevents cell division by disrupting microtubules. The antibody binds to CD30, often found highly expressed on the surface of Hodgkin lymphoma and ALCL cells, and is then internalised where the drug is detached from the antibody and exerts its cellular effects. By preventing cell division it kills cancer cells by the induction of programmed cell death.

Cetuximab (Erbitux) is a chimeric IgG1 monoclonal antibody that targets the extracellular domain (part of the receptor outside the cell) of the epidermal growth factor receptor (EGFR). It is used in the treatment of colorectal cancer and head and neck cancer. Once a ligand binds to the EGFR on the surface of the cell, signalling pathways are activated inside the cell that are associated with malignant characteristics. These include the PI3K/AKT and KRAS/BRAF/MEK/ERK pathways that cause cancer cell proliferation, invasion, differentiation and cancer stem cell renewal. Cetuximab functions by competitively inhibiting ligand binding, thereby preventing EGFR activation and subsequent cellular signalling.

Gemtuzumab ozogamicin is an “immuno-conjugate” of an IgG4 anti-CD33 antibody chemically linked to a cytotoxic calicheamicin derivative, and may be used for the treatment of acute myeloid leukaemia (AML).

Ibritumomab tiuxetan (Zevalin) is a murine anti-CD20 antibody chemically linked to a chelating agent that binds the radioisotope yttrium-90 (90Y). It is used to treat a specific type of non-Hodgkin lymphoma, follicular lymphoma, which is a tumour of B-cells.

Ipilimumab (Yervoy) is a human IgG1 antibody that binds the surface protein CTLA4 which has a role in negatively regulating the activation of T-cells. CTLA4 is discussed below in the context of checkpoint inhibitors.

Nimotuzumab is a chimeric human-mouse anti-EGFR monoclonal antibody and has been approved for squamous cell carcinoma in head and neck (SCCHN).

Ofatumumab is a second generation human IgG1 antibody that binds to CD20. It is used in the treatment of chronic lymphocytic leukemia (CLL) as the cancerous cells of CLL are usually CD20-expressing B-cells. Unlike Rituximab, which binds to a large loop of the CD20 protein, Ofatumumab binds to a separate small loop.

Panitumumab (Vectibix) is a human IgG2 antibody that binds to the EGF receptor. Like Cetuximab, it prevents cell signalling by the receptor by blocking the interaction between the receptor and its ligand. It is used in the treatment of colorectal cancer.

Rituximab is a chimeric monoclonal IgG1 antibody specific for CD20, developed from its parent antibody Ibritumomab. As with Ibritumomab, Rituximab targets CD20, which is present on B-cells. For this reason it is effective in treating certain types of malignancies that are formed from cancerous B-cells. These include aggressive and indolent lymphomas such as diffuse large B-cell lymphoma and follicular lymphoma, and leukaemias such as B-cell chronic lymphocytic leukaemia.

Tositumomab was a murine IgG2a anti-CD20 antibody covalently bound to radioactive Iodine 131 known as “Bexxar” that was approved for treatment of Non-Hodgkin lymphoma, but was voluntarily withdrawn from the market.

Trastuzumab (Herceptin) is a monoclonal IgG1 humanized antibody specific for the epidermal growth factor receptor 2 protein (HER2). It received FDA-approval in 1998, and is clinically used for the treatment of breast cancer. HER-2 is a member of the epidermal growth factor receptor (EGFR) family of transmembrane tyrosine kinases. In a preferred embodiment of the invention the immunotherapeutic agent, and hence anticancer agent, is trastuzumab, preferably for the treatment of ovarian cancer.

In an alternative embodiment the antibody is an anti-CD47 antibody, i.e. an antibody which blocks CD47 signalling. Such antibodies have been shown to eliminate or inhibit the growth of a wide range of cancers and tumours in laboratory tests on cells and mice. CD47 is present on many cancer cells and on many healthy cells.

In a further alternative embodiment the antibody is an antibody to a carbohydrate molecule found on the surface of cancer cells. By way of example, such an antibody may be an anti-GD2 antibody. GD2 is a ganglioside found on the surface of many types of cancer cell including neuroblastoma, retinoblastoma, melanoma, small cell lung cancer, brain tumours, osteosarcoma, rhabdomyosarcoma, Ewing's sarcoma, liposarcoma, fibrosarcoma, leiomyosarcoma and other soft tissue sarcomas. It is not usually expressed on the surface of normal tissues, making it a good target for immunotherapy to allow for specific action against the tumour and reduced toxicity. In further embodiments the therapeutic agents may be used in the treatment or prevention of bone disorders. Such disorders may include any disorder of or involving the bone, including both neoplastic disorders as discussed above (namely cancers of or involving bone), and non-neoplastic disorders. As discussed above the receptor QRFPR has been shown to be expressed on osteoblasts and knock-out of QRFPR is associated with a reduction in the number of osteoclasts. In line with the observations of QRFPR expression in clinical material from Ewing and osteosarcoma the level of QRFPR in osteosarcoma U2OS cells has also been detected and quantified in the present application, and was found to be modulated by stimulation with GDF15.

Accordingly, in a preferred embodiment the binding agent is capable of binding to QRFPR. The polypeptide may be obtained or derived from either receptor but in one embodiment is obtained or derived from QRFPR.

In particular, the bone disorder may be a disorder associated with bone resorption, e.g. increased, excessive or unwanted bone resorption. In one embodiment the bone disorder may be osteoporosis, particularly age-related osteoporosis. In another embodiment, the bone disorder may be osteopenia. The subject may be any human or non-human animal, preferably a mammalian animal subject. In a particular embodiment, the subject is human, but in other embodiments it may be a domestic, livestock, farm, zoo, wild, laboratory or sport (e.g. race) animal, e.g. a primate, dog, cat, murine, pig, cow, horse etc.

As described above, GDF15 binds independently to QRFPR and CLPTM1. GDF15 has been found to induce the loss of QRFPR from the cell surface via exosome shedding and endosomal targeting to the proteasome, and GDF15 has been found to increase the phosphorylation of GSKB via the TGF-β receptor complex. It is therefore possible to determine the ‘activity’ of GDF15 with respect to each of these receptors, i.e. the degree to which GDF15 is able to effect its biological function when it is exposed to a cell, by measuring the loss of QRFPR from the cell surface or by measuring the degree to which GSKB is phosphorylated. The ability of a binding agent or polypeptide of the present invention to reduce the activity of GDF15 (i.e. to prevent GDF15 from effecting or exerting its biological function) may thus be determined, by measuring whether a particular binding agent or peptide is able to reduce the activity of GDF15. A binding agent or polypeptide of the present invention will thus preferably be able to bind to a GDF receptor or to GDF15, and substantially inhibit or abrogate the activity of GDF15, i.e. to reduce the activity of GDF15 to less than 50%, 40%, 30%, 20% or 10% compared to the level of activity of GDF15 in the absence of a binding agent or polypeptide. More preferably, the binding agent or polypeptide will reduce the activity of GDF15 to less than 9%, 8%, 7%, 6%, 5%, 4%, 3%, 2%, 1% or less, i.e. the binding agent or polypeptide may preferably effectively eliminate the activity of GDF15.

In addition to the binding agents and proteins or peptides, a therapeutic composition may comprise any pharmaceutically acceptable diluent, carrier or excipient. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, pH, temperature etc.

Dosages of the therapeutic agents may also be determined in routine manner according to standard clinical practice. Doses of 0.1-10 mg/kg, such as doses of 0.1-0.5 mg/kg, 0.1-1 mg/kg, 0.1-2 mg/kg, 0.1-5 mg/kg, 0.5-1 mg/kg, 0.5-2 mg/kg, 0.5-5 mg/kg, 0.5-10 mg/kg, 1-2 mg/kg, 1-5 mg/kg, 1-10 mg/kg, 2-5 mg/kg, 2-10 mg/kg or 5-10 mg/kg may be administered daily, weekly, or every 10 days, 2 weeks, 3 weeks or monthly until disease progression, or until unacceptable toxicity is observed.

Likewise the therapeutic agents may be administered in any convenient or desired manner, e.g. parenterally or non-parenterally, for example by enteral administration, e.g. orally (depending on the nature and/or formulation of the agent), or by intravenous, sub-cutaneous, intramuscular, intraperitoneal injection or infusion. The administration may be systemic or local, depending e.g. on the condition to be treated, the nature of the agent and/or formulation etc. Thus, for example the agent may be delivered locally e.g. by infusion or direct injection, e.g. to the site or location of a cancer.

The interaction between GDF15 and the receptor QRFPR and/or CLPTM1, or an effect of the binding of GDF15 to one or both of the receptors, may be detected and such a detection method may be used as the basis of a companion diagnostic for the therapeutic or prophylactic methods disclosed herein.

Thus, the detection of an interaction and/or an effect thereof may be used to detect or determine that, or whether, a subject may be in need of therapy or prophylaxis (prevention) according to the present invention, or may benefit therefrom, or to monitor or assess the therapy or prophylaxis, e.g. during the course of or at the end of treatment. Thus the presence of an interaction of GDF15 with the receptor QRFPR and/or CLPTM1, or the presence of a detectable effect of GDF15 at the receptor QRFPR and/or CLPTM1, may be used as a biomarker, e.g. a predictive biomarker for administration of a therapy or prophylaxis according to the present invention, or to monitor or assess the effect of the therapy or prophylaxis. Further, by detecting an interaction and/or an effect thereof, it may be determined or detected whether a subject has a condition associated with an elevated GDF15 level, e.g. a cancer, particularly a bone cancer, including a primary bone cancer or a metastasis to bone. In particular, it may be used to detect a metastasis to bone.

Accordingly, a further aspect of the present invention provides a method of detecting a subject in need of therapy or prophylaxis, particularly by administration of a therapeutic agent according to the present invention (that is a polypeptide and/or binding agent which is capable of inhibiting an interaction of GDF15 with the receptor QRFPR and/or CLPTM1, e.g. an agent as defined herein), said method comprising detecting an interaction between GDGF15 and the receptor QRFPR and/or CLPTM1 and/or an effect of such an interaction in the subject.

In another aspect the invention provides a method of assessing or monitoring a method of therapy or prophylaxis by administration to a subject of a therapeutic agent according to the present invention (that is a polypeptide and/or binding agent which is capable of inhibiting an interaction of GDF15 with the receptor QRFPR and/or CLPTM1, e.g. an agent as defined herein), said method comprising detecting and/or monitoring an effect of such an interaction in the subject.

A still further aspect provides a method of detecting a subject having, or at risk of developing, a condition associated with an elevated level of GDF15, said method comprising detecting in said subject an interaction of GDF15 with the receptor QRFPR and/or CLPTM1, or an effect of a said interaction.

The interaction or an effect thereof may be detected in vivo (i.e. in a subject) or in vitro. That is, the interaction or an effect thereof may be detected in the body of a subject or, preferably, in a sample from said subject. The sample may be any appropriate clinical sample. More particularly the sample may be a sample comprising cells expressing the receptor QRFPR and/or CLPTM1. The sample may thus be any cell-containing clinical sample from the subject. It may be a sample of tissue or body fluid, e.g. a tissue biopsy sample, blood or a blood-derived sample (e.g. plasma, serum, or a fraction thereof), urine, CSF, saliva, stool, or a swab, washing or rinsate etc.

An interaction may be detected by any means known in the art for detecting binding or interaction between two or more binding partners. Thus the method may be any method based on detecting binding between GDF15 and a receptor. It may for example be a proximity assay-based method, and in particular a proximity assay based on using antibody-based binding partners for the GDF15 and a receptor. Proximity assays are well known in the art and widely described in the literature. Proximity assays based on pairs (or more) of proximity probes each comprising an binding partner capable of binding directly or indirectly to an analyte (e.g. via an intermediate analyte-binding antibody or other binding partner for the analyte) and a nucleic acid domain which interacts with the nucleic acid domain of the other proximity probe(s) to generate a detectable signal when the probes have been bound in proximity (e.g. when the partners of an interacting pair have interacted or bound together) have been developed and commercialised by Olink AB of Uppsala, Sweden. The interaction between the nucleic acid domains of proximity probes may comprise a nucleic acid ligation and/or extension reaction and may be detected by detecting a ligation and/or extension product. The nucleic acid domains themselves may interact (e.g. may be ligated together), or they may template the formation of a ligation and/or extension product from one or more added oligonucleotides. Particular mention may be made of an in situ proximity ligation assay (PLA) which may be used to detect an interaction in situ in a cell or tissue sample. Such an assay has been developed by Olink AB and is marketed under the Duolink® brand name. Proximity assay are described in U.S. Pat. No. 6,878,515, U.S. Pat. No. 7,306,904, WO 2007/107743, WO/EP2012/051474 and WO2012/152942.

Further methods for detecting binding between GDF15 and a receptor may include immunoassays, such an enzyme-linked immunoassay (ELISA), radioimmunoassay (RIA) or immuno-PCR.

Various effects of GDF binding to QRFPR or CLPTM1 are described above and any of these may be detected. In a particular preferred embodiment the generation of QRFPR-positive exosomes may be detected. QRFPR-positive exosomes may be detected, for example, in conditioned medium obtained from cells, e.g. by Western blot. Alternatively, QRFPR-positive exosomes may be detected by microscopy, e.g. fluorescence or electron microscopy. The interaction of GDF15 with QRFPR-positive exosomes may also be detected, e.g. by any of the proximity-based detection assays mentioned above. In particular, in situ PLA may be used. In a further preferred embodiment, phosphorylated GSK3b, generated through activation of CLPTM1 by GDF15, may be detected. In one embodiment, this may be done using a proximity-based detection assay, e.g. by using a first probe capable of binding to GSK3b and a second probe capable of binding to a phosphate group. In one embodiment, detection may be performed by PLA, in particular by in situ PLA.

The present invention may be better understood through the following Examples and Figures, in which:

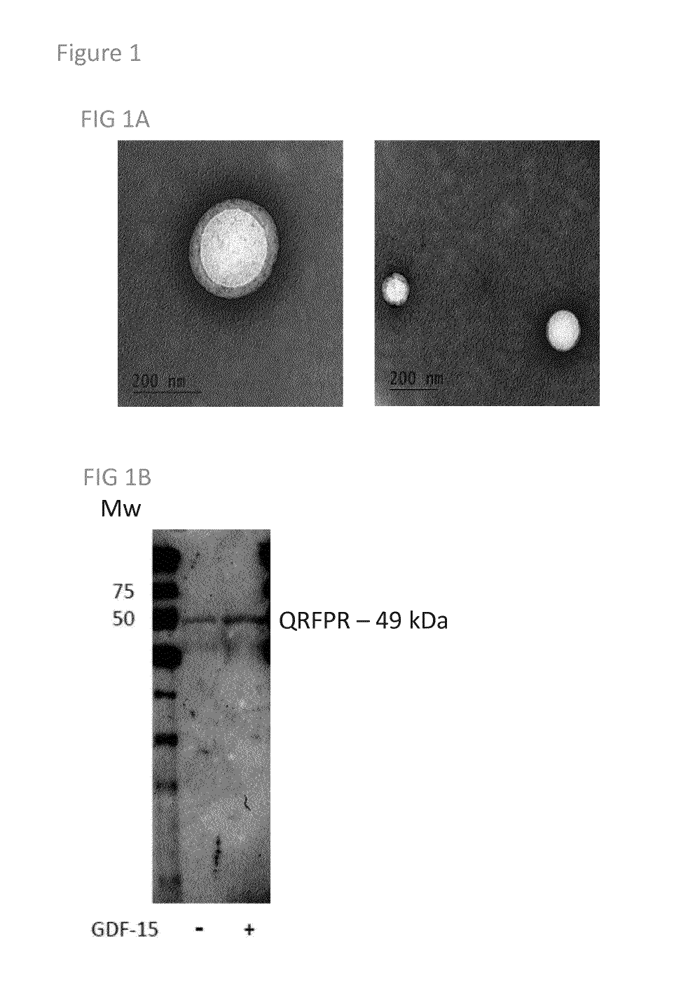

FIG. 1 shows that exosomes comprising QRFPR are released by cells exposed to GDF15. A) Electron micrographs of exosomes obtained from conditioned medium obtained from MCF-7 breast cancer cells exposed to GDF15. B) Western blot of exosomes obtained from conditioned medium from MCF-7 cells—QRFPR is detected as a single band at 49 kDa. Cells exposed to GDF15 showed higher levels of QRFPR in conditioned medium than cells that not been exposed to GDF15.

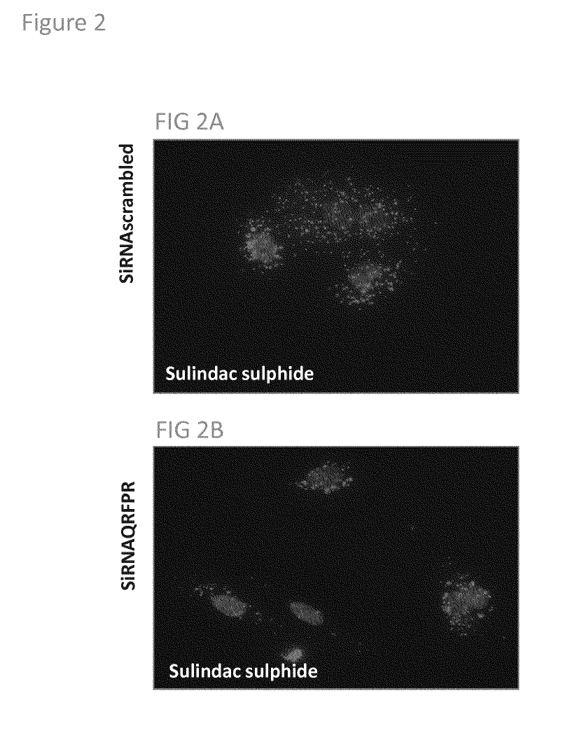

FIG. 2 demonstrate the GFD15-QRFPR complex on the cell surface. A-B) In situ PLA detection of the GDF15-QRFPR complex at the surface of MCF-7 cells. A) Cells following exposure to Sulindac sulphide. Cells treated with siRNA control (scrambled RNA). B) Cells following exposure to Sulindac sulphide and treatment with QRFPR siRNA to reduce the level of QRFPR present in the cells.

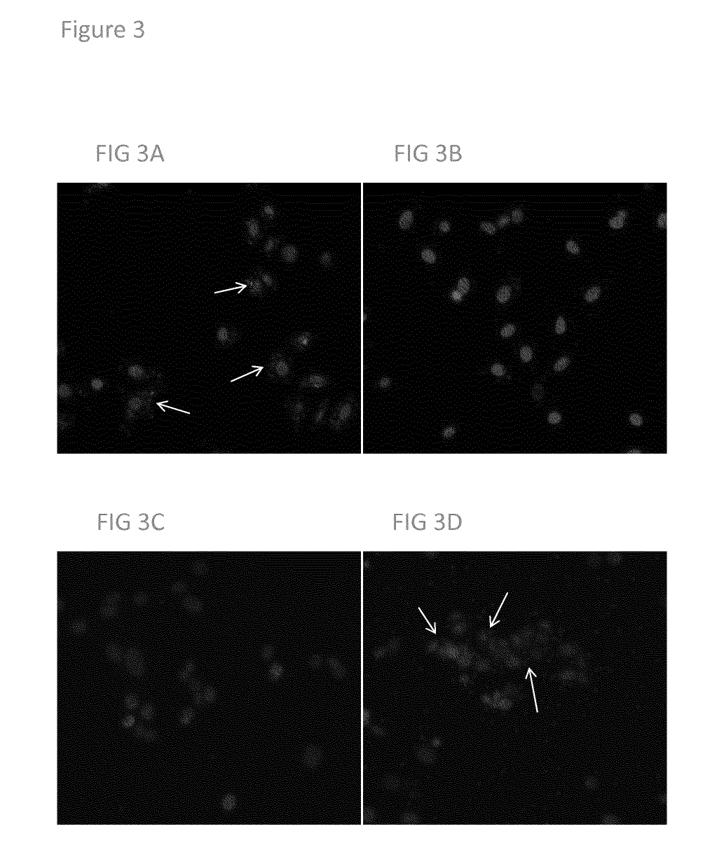

FIG. 3 shows GDF15 stimulation reduces the level of the ACTRIIB-QRFPR complex at the cell surface, and that Early Endosome Antigen 1 (EEA1) is recruited to QRFPR by GDF15. A-B) In situ PLA detection of the ACTRIIB-QRFPR complex on the surface of MCF-7 cells. Stimulation of cells with GDF15 for 20 minutes at 37° C. reduced surface levels of the complex. C-D) In situ PLA detection of the QRFPR-EEA1 complex on the surface of MCF-7 cells. Stimulation of cells with GDF15 for 5 minutes at 37° C. resulted in the recruitment of EEA1 to QRFPR.

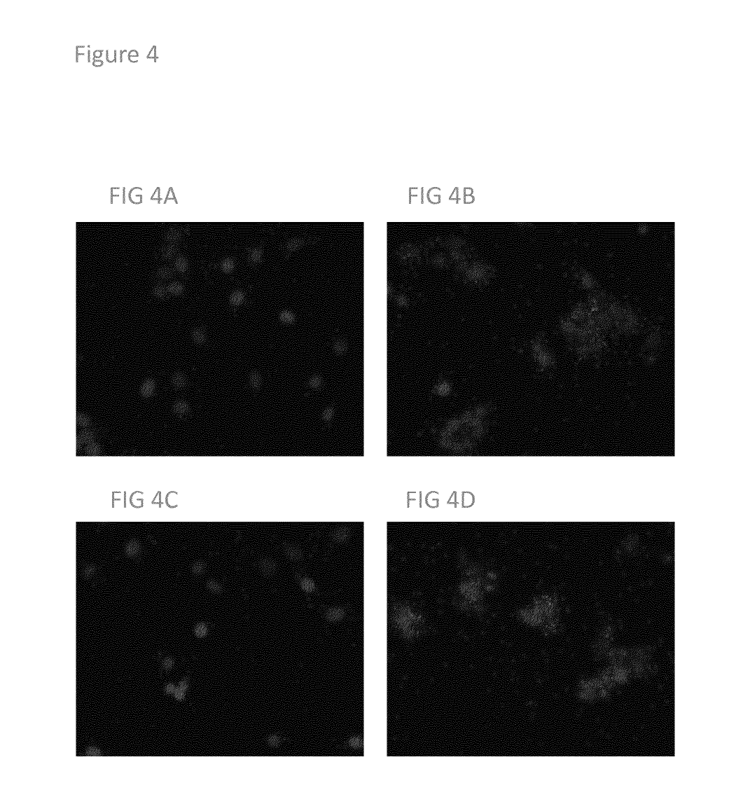

FIG. 4 shows that GDF15 stimulation results in the formation of the CD9-QRFPR complex at the cell surface. A-D) In situ PLA detection of the CD9-QRFPR complex on the surface of MCF-7 cells. A) & C) Negative control cells with no added GDF15. B) & D) Cells after exposure to GDF15 for 30 minutes at 37° C. The CD9-QRFPR complex is detected at a higher level in cells stimulated with GDF15 for 30 minutes.

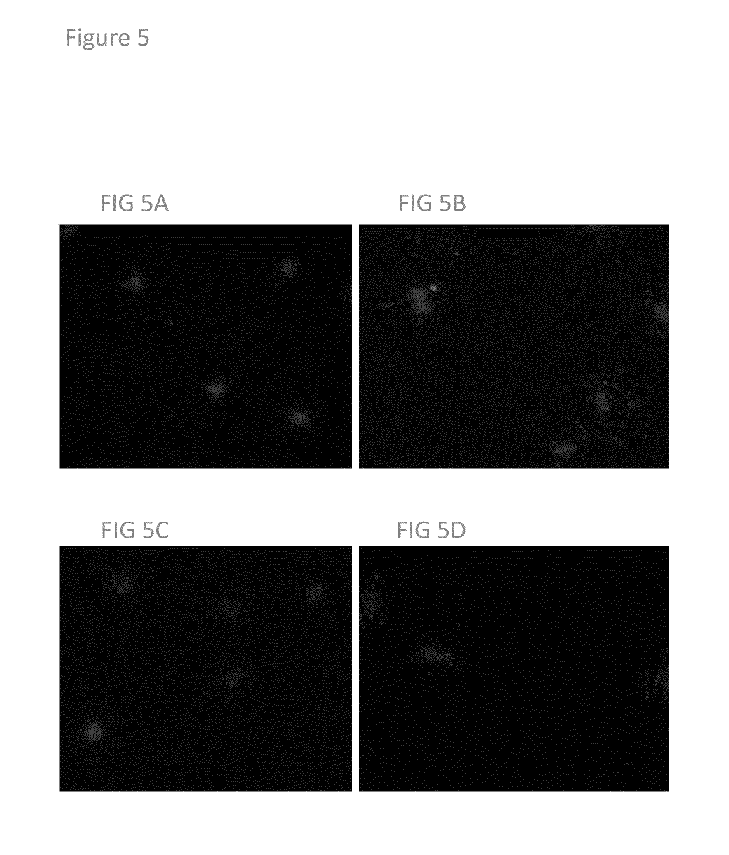

FIG. 5 shows that GDF15 stimulation results in the formation of Rab11-QRFPR double-positive endosomes. A-D) In situ PLA detection of the Rab11-QRFPR complex in MCF-7 cells. A) & C) Negative control cells with no added GDF15. B) & D) Cells after exposure to GDF15. The Rab11-QRFPR complex is detected at a higher level in cells stimulated with GDF15.

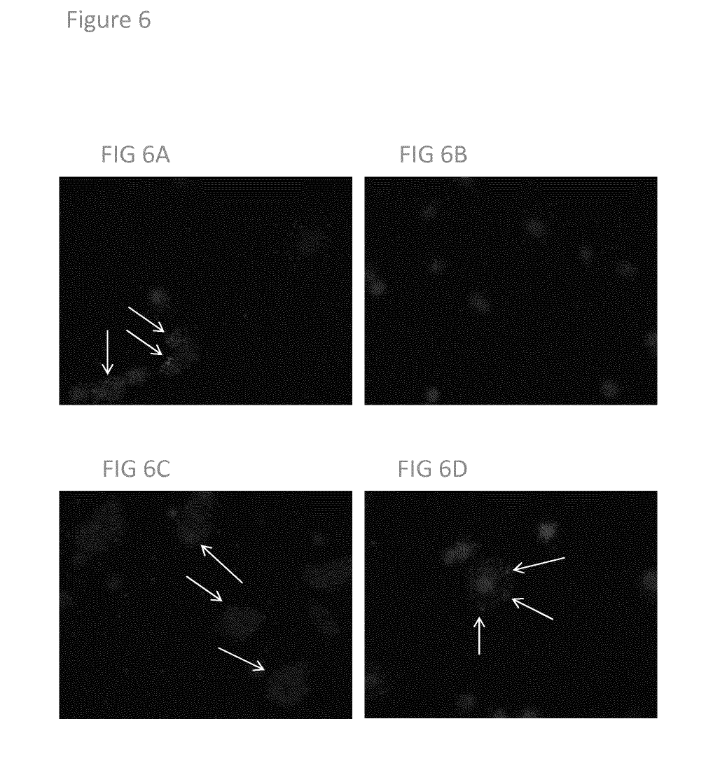

FIG. 6 shows that GDF15 stimulation reduces the level of QRFPR in cells and targets QRFPR for proteasomal degradation. A-D) Detection of QRFPR at the cell surface. A) Control cells with no added GDF15. B) Cells after exposure to GDF15 o/n. C) Control cells treated with proteasomal inhibitor MG132 o/n. D) Cells after exposure o/n to GDF15, in the presence of proteasomal inhibitor MG132. Levels of QRFPR are reduced following exposure to GDF15 in the absence of the MG132 (B), however, levels of QRFPR are not affected by GDF15 in the presence of MG132, indicating that the degradation of QRFPR induced by GDF15 is blocked.

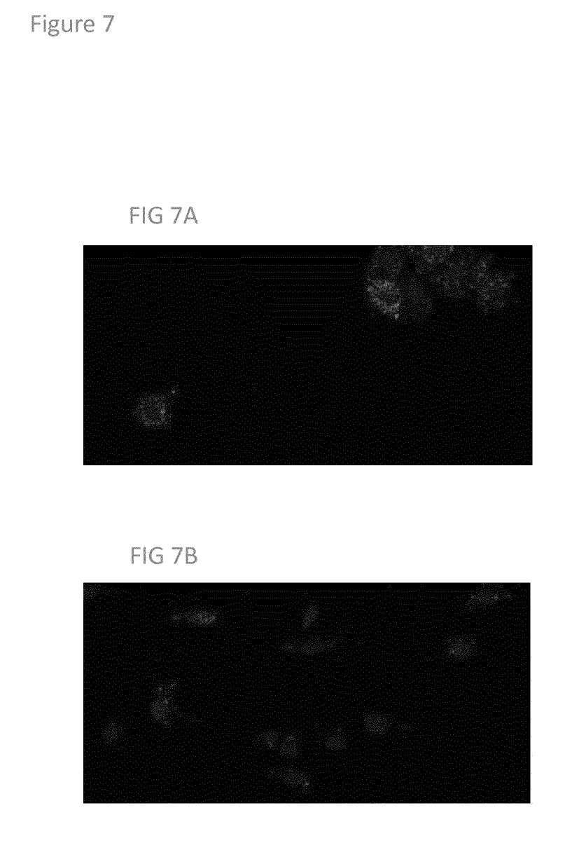

FIG. 7 shows that GDF15 stimulation results in the formation of Rab4-QRFPR double-positive endosomes. A) In situ PLA detection of the Rab4-QRFPR complex following exposure of cells to GDF15. B) In situ PLA detection of the Rab4-QRFPR complex following exposure of cells to GDF15, where cells were pre-incubated with an antibody which binds to an extracellular domain of QRFPR. The level of the Rab4-QRFPR complex detected in cells pre-incubated with the blocking antibody is reduced.

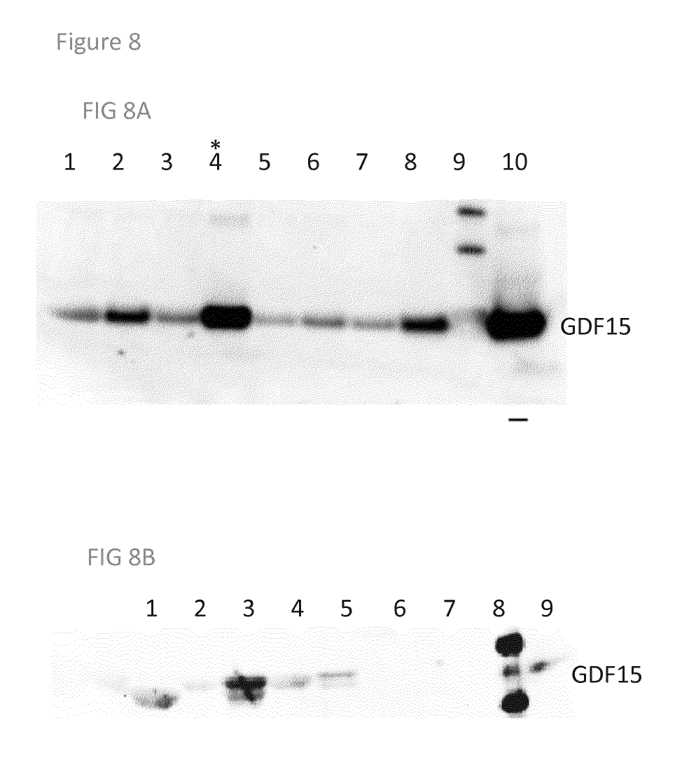

FIG. 8 shows that peptides derived from QRFPR and CLPTM1 are able to interact with GDF15. A) Biotin-labelled peptides from the extracellular domains of QRFPR and CLPTM1 were immobilised on Streptavidin-agarose beads and were found to found to interact with GDF15 after extensive washes. B) GST fusion proteins comprising a peptide derived from the extracellular domains of QRFPR and CLPTM1 were incubated with GDF15. Fusion proteins comprising these peptides were found to interact with GDF15.

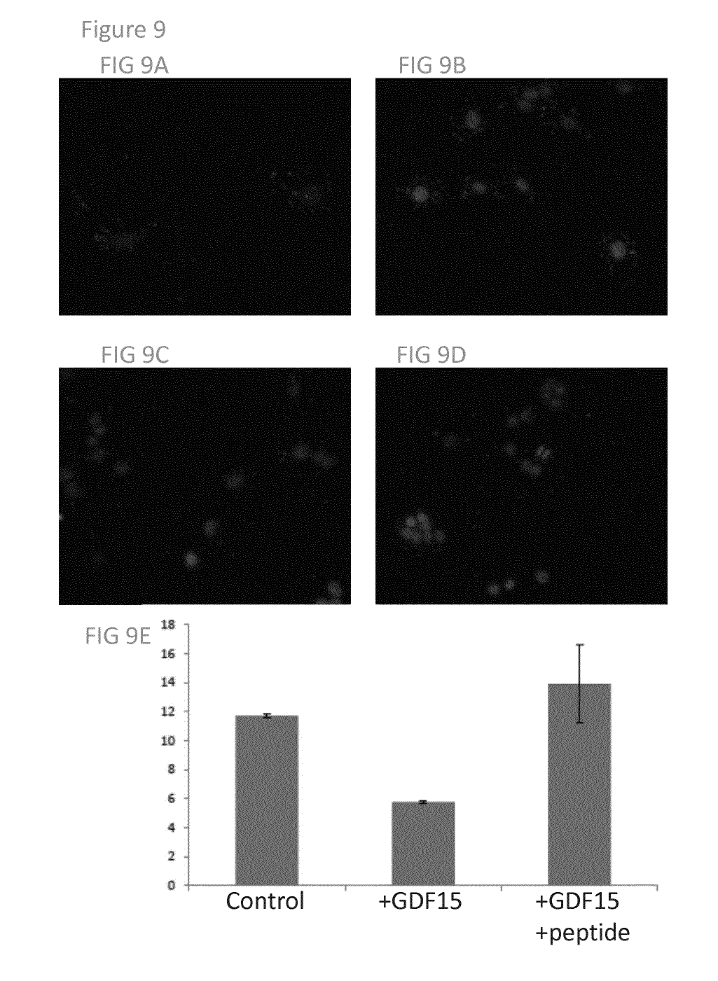

FIG. 9 shows that a peptide from an extracellular loop of QRFPR is sufficient to block GDF15 mediated endosomal accumulation of QRFPR. A-D) In situ PLA detection of the Rab11-QRFPR complex following exposure of MCF7 cells to GDF15. A) +Ctrl peptide. B) +QRFPR extracellular N-terminal peptide. C-D) +QRFPR extracellular loop3 (domain 4) peptide. The level of the Rab11-QRFPR complex is reduced by the peptide from extracellular loop3 of QRFPR. E) in situ PLA detection of QRFPR in U2OS osteosarcoma cells. Bar graph indicating that the peptide from extracellular loop 3 of QRFPR is able to restore the level of QRFPR detected at the cell surface to the same level as control cells that had not been exposed to GDF15.

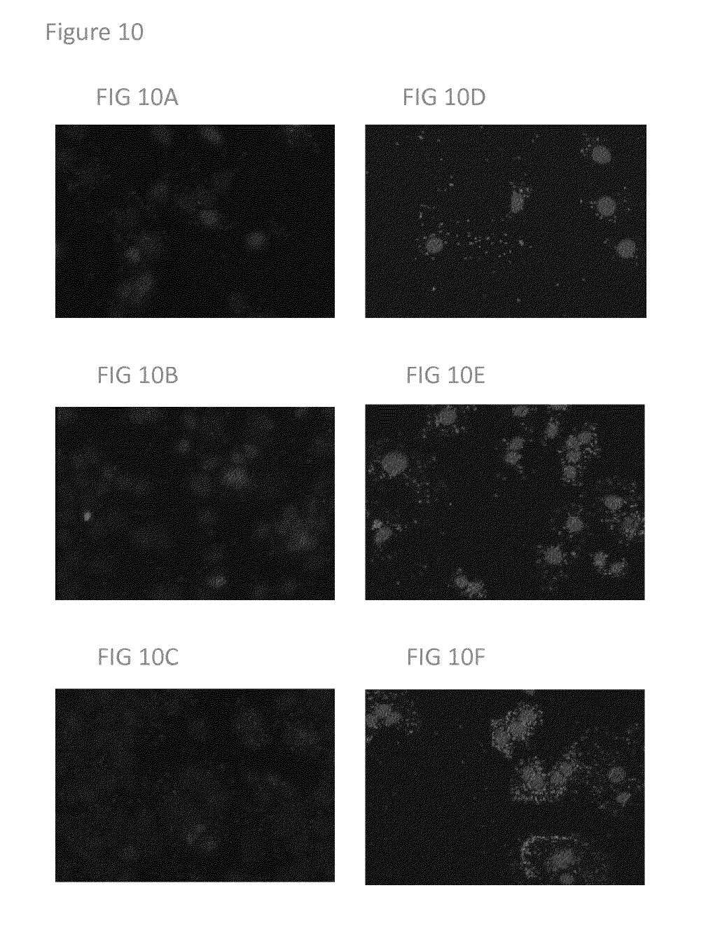

FIG. 10 shows that a peptide from an extracellular loop of QRFPR may reduce the effect of GDF15 on QRFPR levels at the cell surface, whilst not affecting activation of QRFPR by its endogenous agonist P518. A-C) In situ detection of QRFPR at the cell surface. A) Negative control cells with no added GDF15. B) Cells after exposure to GDF15. C) Cells after exposure to GDF15+ synthetic peptide GEKEYDDVTIK derived from extracellular domain 4 of QRFPR. The peptide reversed the removal of QRFPR from the cell surface. D-F) In situ PLA detection of ACTRIIB at the cell surface. D) Control cells with no added P518. E) Cells after exposure to P518. F) Cells after exposure to P518+ synthetic peptide GEKEYDDVTIK derived from extracellular domain 4 of QRFPR in 125 molar excess. The ability of P518 to increase the level of ACTRIIB at the cell surface was not affected by the peptide.

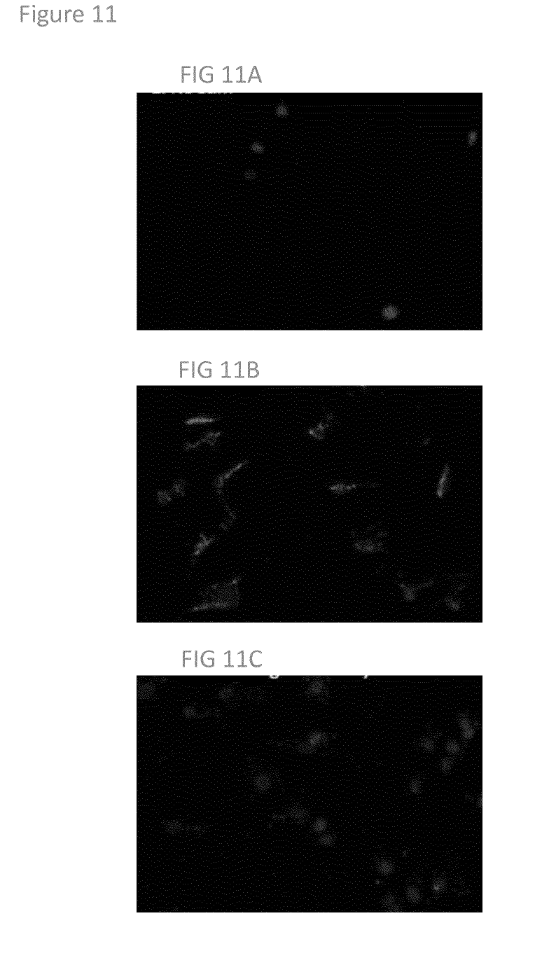

FIG. 11 shows that antibodies targeting the extracellular domain of QRFPR are capable of preventing GDF15-induced endocytosis. A-C) In situ PLA detection of the Rab4-QRFPR complex. A) Negative control cells with no added GDF15. B) Cells after exposure to GDF15. C) Cells after exposure to GDF15 following pre-incubation with an antibody targeting the extracellular domain of QRFPR.

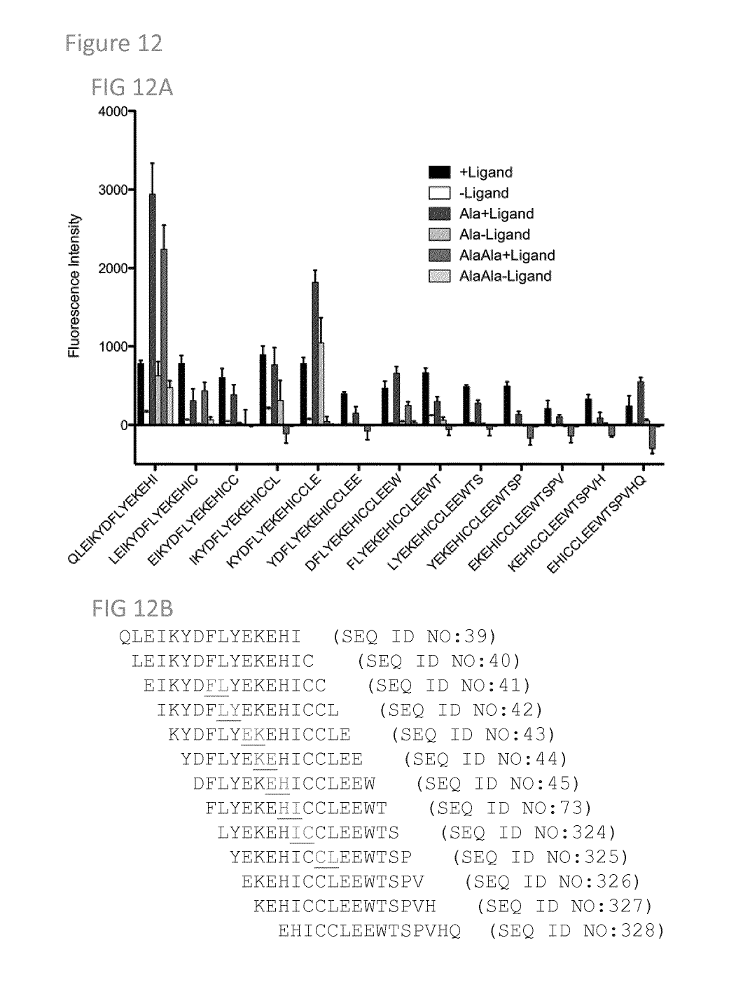

FIG. 12 shows combined results of a peptide screen to identify residues within the third extracellular domain of QRFPR that are required for binding to GDF15 measuring binding on a peptide array. A) Results of binding of GDF15 to immobilised peptides. B) Alignment of peptides tested in peptide array. Underlined residues indicate a reduction in binding when substituted to alanine or double-substitution (AA). SEQ ID numbers for the peptides used in the screen are shown.

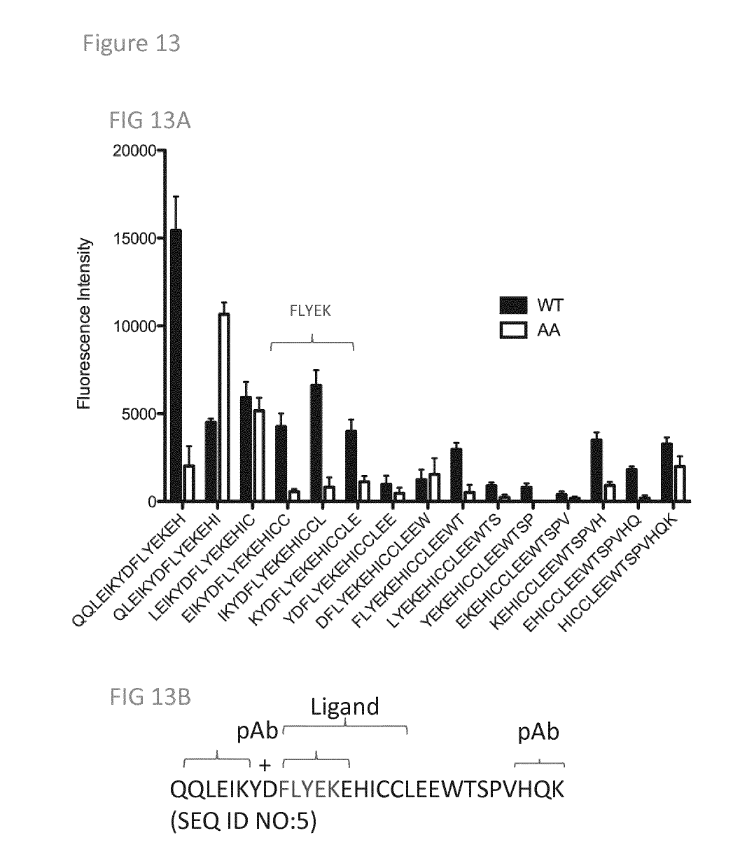

FIG. 13 shows the identification of the epitopes of polyclonal antibodies targeting the extracellular domain of QRFPR by measuring binding of the antibodies to a series of peptides. A) Results of binding pAb to immobilised peptides. B) Summary of binding results indicating epitopes of the pAb relative to the position of the ligand binding site. The epitope of one of the antibodies overlays well with the ligand binding site. Sequences of the amino acids used in the screen are shown in Example 8.

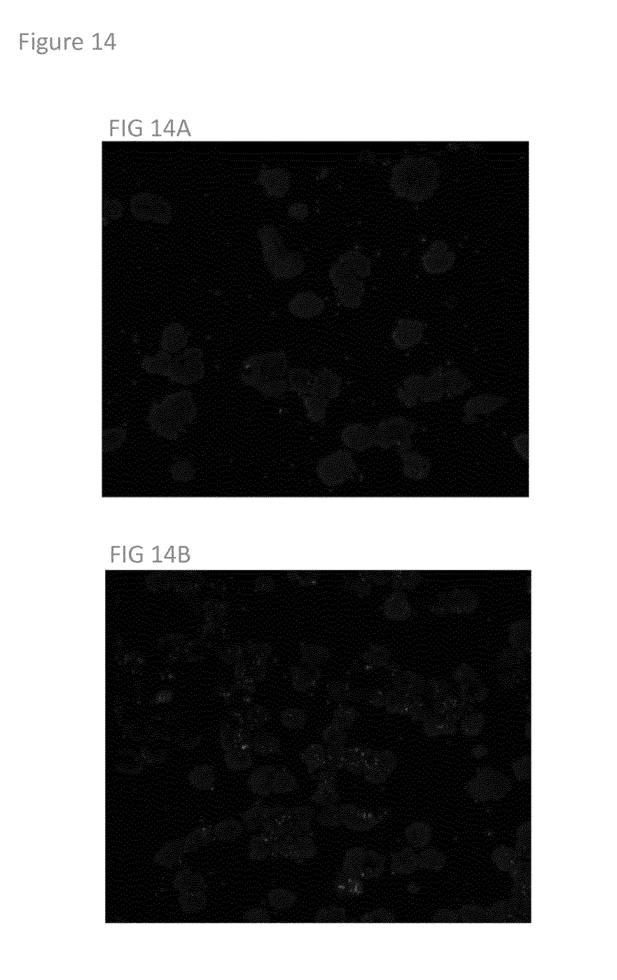

FIG. 14 shows that GDF15 treatment of NK cells expressing CLPTM1 increases the level of the CLPTM1-TGFBRI complex. A-B) In situ PLA detection of the CLPTM1-TGFBRI complex. A) Negative control cells with no added GDF15. B) Cells after exposure to GDF15. The level of the CLPTM1-TGFBR1 complex increases in response to GDF15.

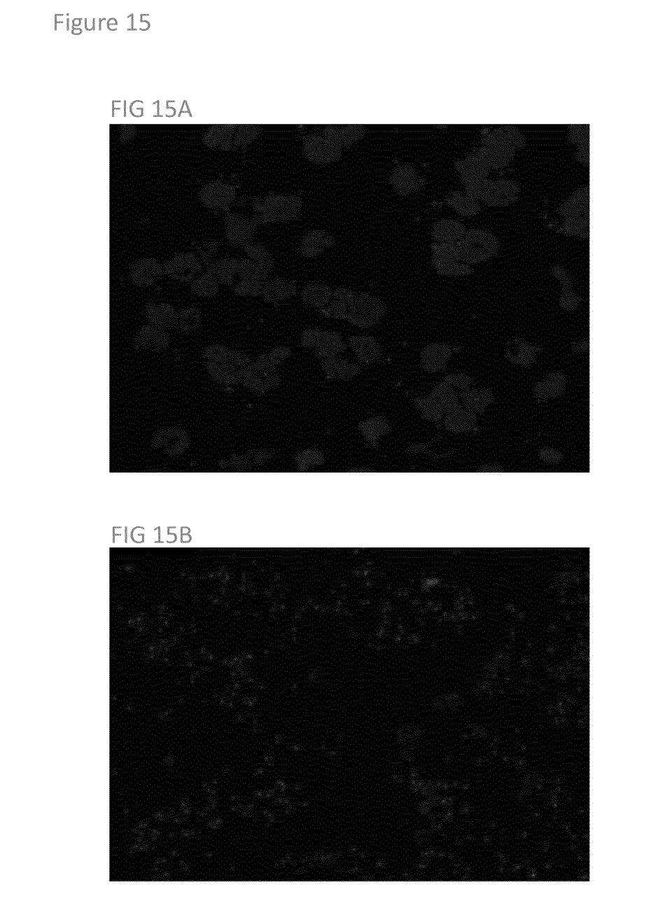

FIG. 15 shows that GDF15 treatment of NK cells expressing CLPTM1 increases the level of the CLPTM1-TGFBRII complex. A-B) In situ PLA detection of the CLPTM1-TGFBRII complex. A) Negative control cells with no added GDF15. B) Cells after exposure to GDF15. The level of the CLPTM1-TGFBRII complex increases in response to GDF15.

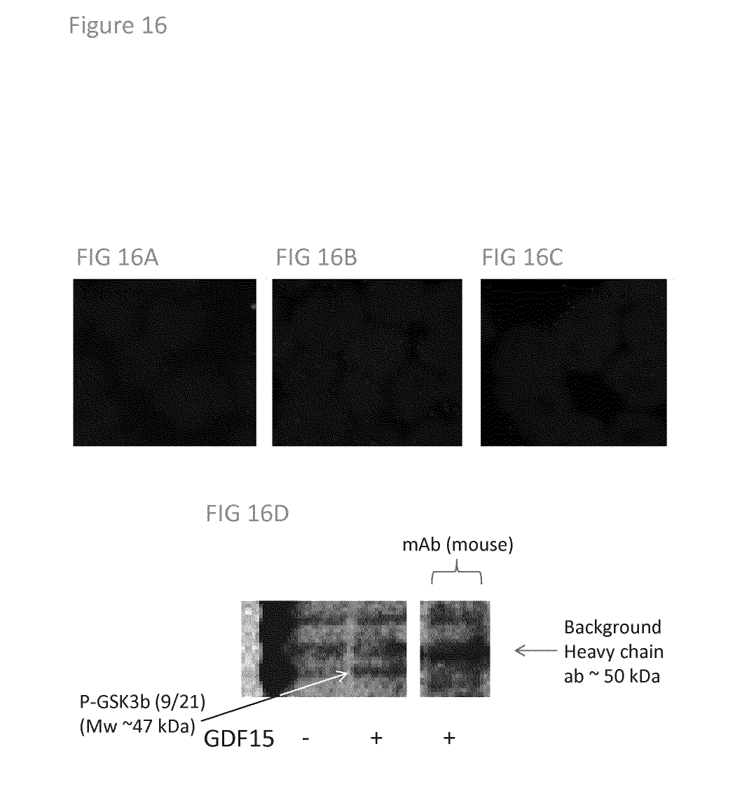

FIG. 16 shows that a monoclonal antibody targeting CLPTM1 is capable of blocking the interaction between GDF15 and CLPTM1 in NK-cells. A-C) In situ PLA detection of phosphorylated GSK3b (GSK3b-p(9/21) using a pair of proximity probes A) Negative control cells with no added GDF15. B) Cells after exposure to GDF15 and control antibody. C) Cells after exposure to GDF15 following pre-incubation with a monoclonal antibody targeting the extracellular domain of CLPTM1 D) Western Blot indicating the levels of phosphorylated GSKb (p-GSKb). Lane 1—No GDF15. Lane 2—GDF15 stim+ ctrl ab. Lane 3 GDF15 stim+ mAb mouse (antibody used in in situ PLA experiment). The mouse mAb antibody is capable of reducing the level of p-GSKb in cells exposed to GDF15.

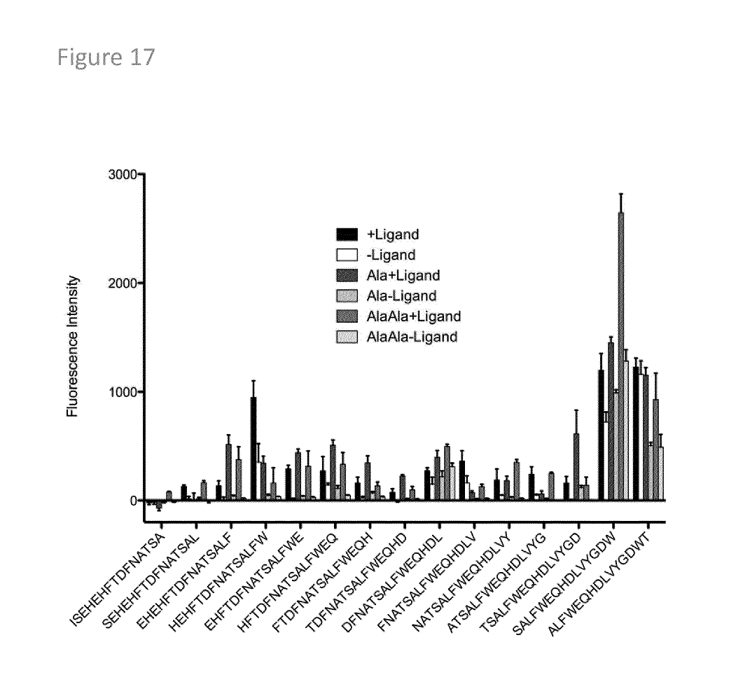

FIG. 17 shows combined results of a peptide screen to identify residues within the extracellular domain of CLPTM1 that are required for binding to GDF15 measuring binding on a peptide array. The results of binding of GDF15 to immobilised peptides are shown.

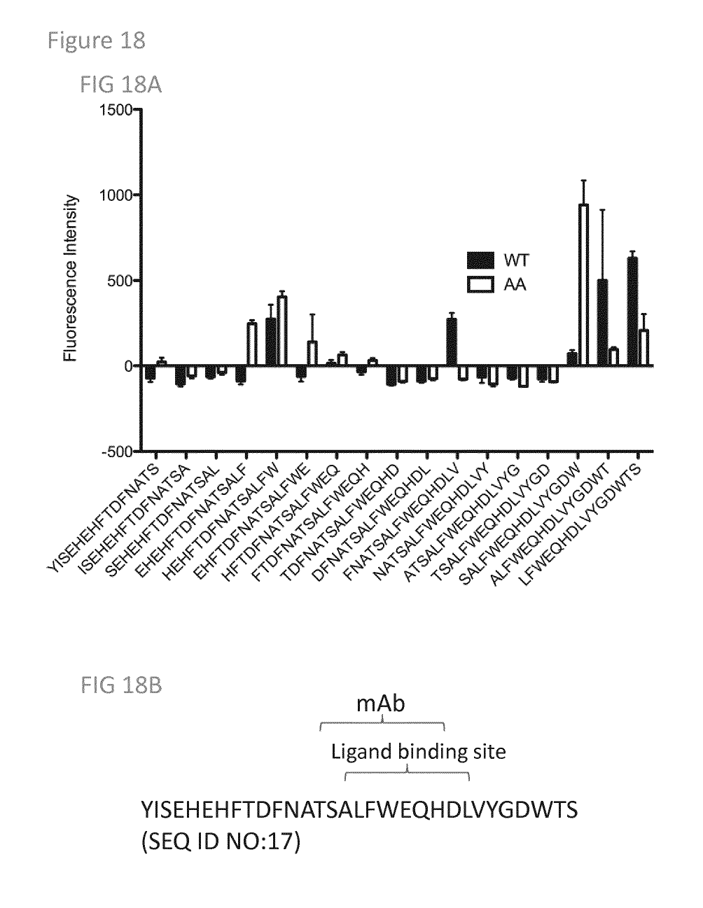

FIG. 18 shows the identification of the epitopes of a monoclonal antibody targeting the extracellular domain of QRFPR by measuring binding of the antibody to a series of peptides. A) Results of binding mAb to immobilised peptides. B) Summary of binding results indicating the epitope of the mAb relative to the position of the ligand binding site. The epitope of one of the antibody overlays well with the ligand binding site.

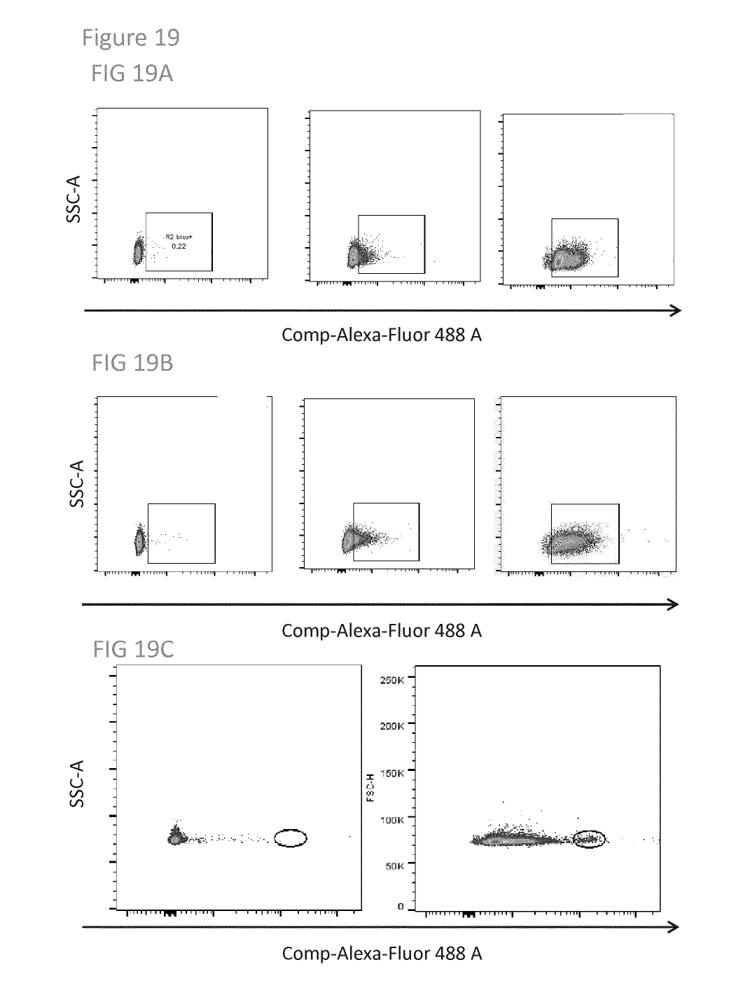

FIG. 19 shows that cells of the immune system express CLPTM1. A) CD4+T-lymphocytes detected by flow cytometry in SSC-A and Alexa-488 channels. Left—isotype control. Middle—0.3 pg anti-CLPTM1-Alexa. Right—3 μg anti-CLPTM1-Alexa. B) CD8+T-lymphocytes detected by flow cytometry in SSC-A and Alexa-488 channels. Left—isotype control. Middle—0.3 μg anti-CLPTM1-Alexa. Right—3 μg anti-CLPTM1-Alexa. C) CD45+/CD3+(Non-T-lymphocytes) detected by flow cytometry in SSC-A and Alexa-488 channels. Left—isotype control. Right—anti-CLPTM1-Alexa. CLPTM1 expression is detected in both CD4+ and CD8+T-lymphocytes and in CD45+/CD3+ non-T-cells.

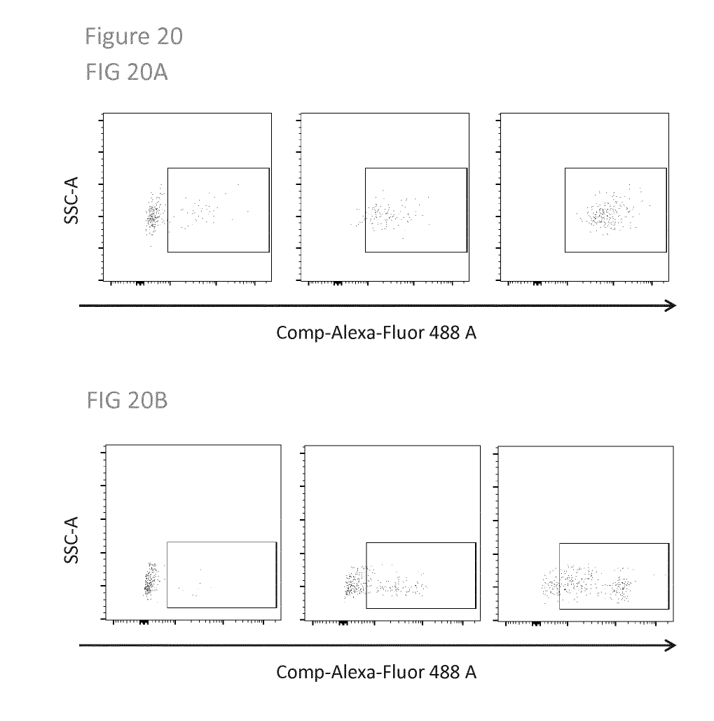

FIG. 20 shows that cells of the immune system express CLPTM1. A) CD14+ monocytes/macrophages detected by flow cytometry in SSC-A and Alexa-488 channels. Left—isotype control. Middle—0.3 μg anti-CLPTM1-Alexa. Right—3 μg anti-CLPTM1-Alexa B) CD11c+ dendritic cells detected by flow cytometry in SSC-A and Alexa-488 channels. Left—isotype control. Middle—0.3 μg anti-CLPTM1-Alexa. Right—3 μg anti-CLPTM1-Alexa.

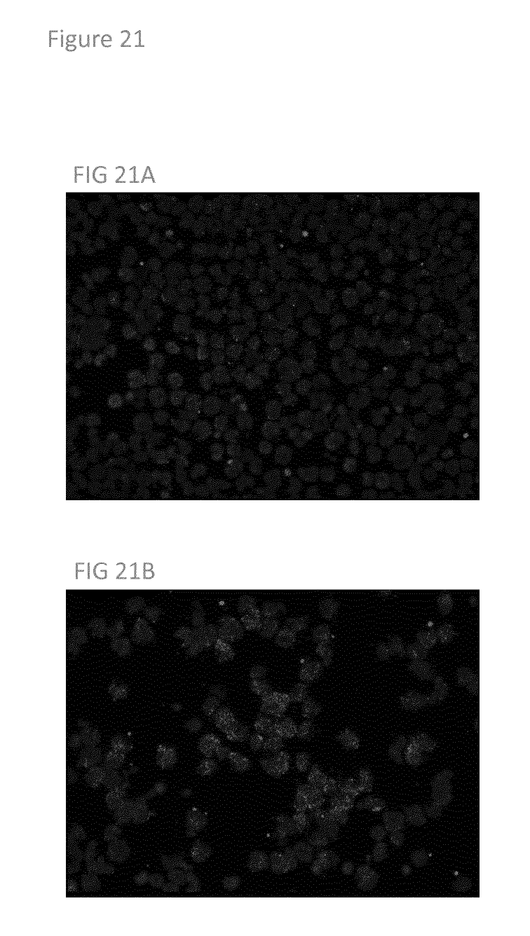

FIG. 21 shows that a monoclonal antibody targeting CLPTM1 is capable of blocking the interaction between GDF15 and CLPTM1 in NK-cells, and inhibiting the down-regulation of NKG2D by GDF15. A-B Detection of NKG2D in NK cells. A) Cells after exposure to GDF15. B) Cells after exposure to GDF15 following pre-incubation with an antibody targeting the extracellular domain of CLPTM1.

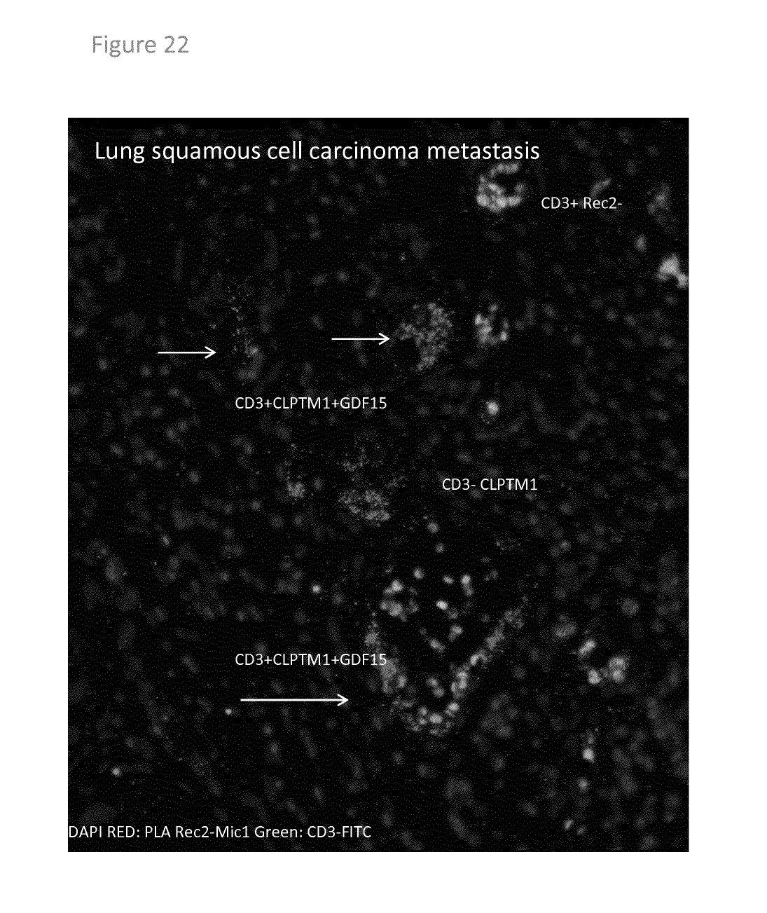

FIG. 22 shows that the CLPTM1-GDF15 complex is found in lung squamous cell carcinoma metastasis. The interaction between CLPTM1 and GDF15 is indicated by arrows. This interaction was found to co-localise with CD3+ cells of the immune system, indicating that CLPTM1 may be important in modulating immune function.

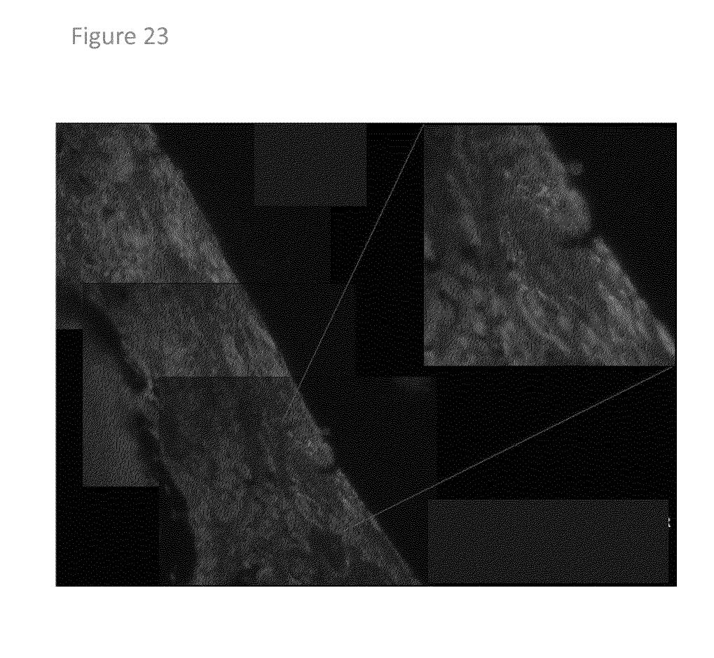

FIG. 23 shows that elevated levels of the GDF15-QRFPR complex are found in prostate cancer micrometastasis to bone. In situ PLA detection of the GDF15-QRFPR complex in prostate micrometastasis to bone indicates that a unique signature is found in the border of prostate cancer micrometastases infiltrating bone.

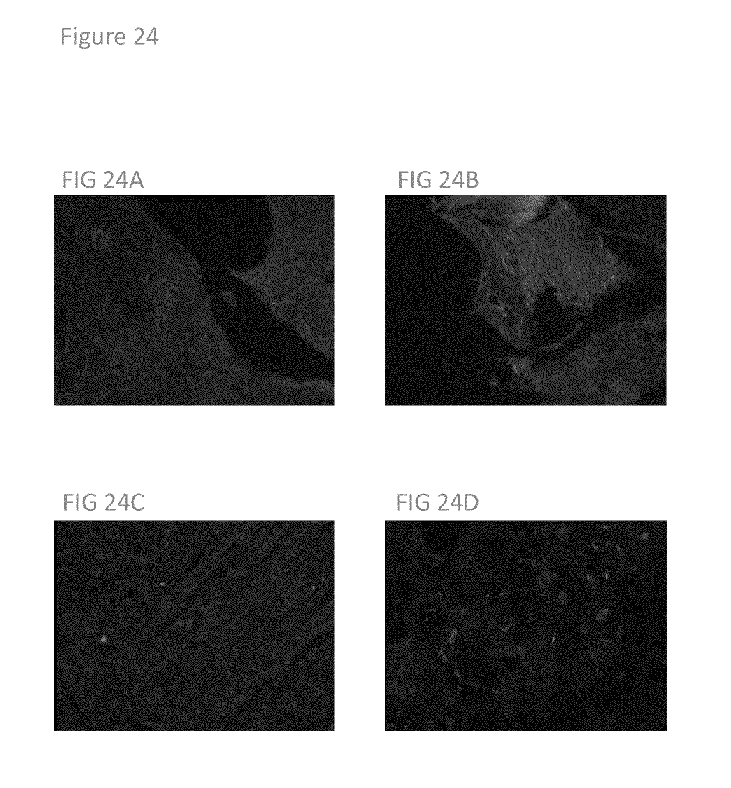

FIG. 24 shows that elevated levels of the GDF15-QRFPR complex are found in osteosarcoma and prostate bone metastases. A-B) In situ PLA detection of the GDF15-QRFPR complex in prostate micrometastasis of bone indicates that an elevated level of the GDF15-QRFPR complex is found in the border of prostate micrometastasis infiltrating bone. C) In situ PLA detection of the GDF15-QRFPR complex in primary prostate cancer. Levels of the GDF15-QRFPR complex in primary prostate cancer were reduced. D) In situ PLA detection of the GDF15-QRFPR complex in primary osteosarcoma indicates that an elevated level of the GDF15-QRFPR complex may be found in primary bone cancer.

FIG. 25 shows that elevated levels of the GDF15-QRFPR complex are found in Ewing sarcoma. A) In situ PLA detection of the GDF15-QRFPR complex in primary bone cancer indicates that an elevated level of the GDF15-QRFPR complex may be found in primary bone cancer. B) In situ detection of the GDF15-CLPTM1 complex in tissue adjacent to Ewing sarcoma (slide of part (A) analysed for QRFPR). No detection of the complex is found, indicating that CLPTM1 is not present.

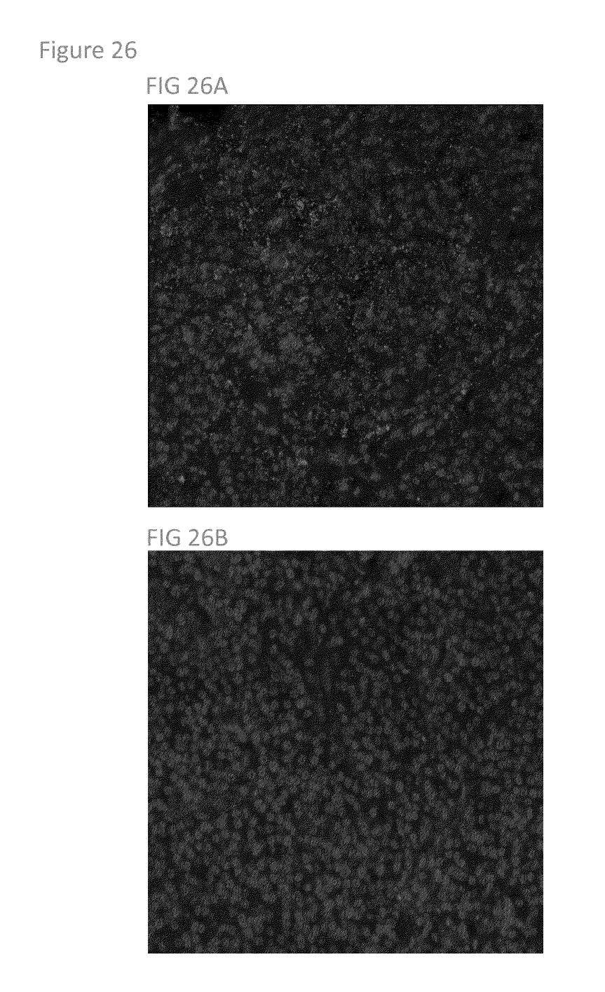

FIG. 26 shows that an elevated level of the GDF15-QRFPR complex is found in malignant myeloma. A) A) In situ PLA detection of the GDF15-QRFPR complex in myeloma indicates that an elevated level of the GDF15-QRFPR complex may be found in myeloma. B) In situ detection of the GDF15-CLPTM1 complex in tissue adjacent to myeloma (slide of part (A) analysed for QRFPR). No detection of the complex is found, indicating that CLPTM1 is not present.

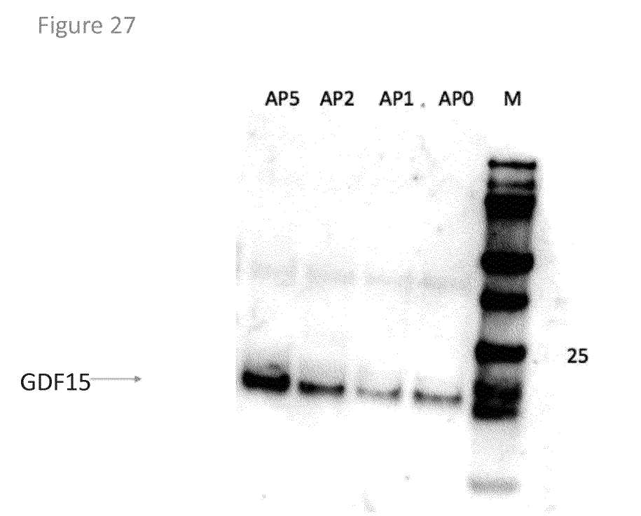

FIG. 27 shows that Fc fusion proteins comprising polypeptides derived from QRFPR and CLPTM1 interact with GDF15 in a pull-down assay with stringent wash conditions. AP5—QRFPR polypeptide, AP2—CLPTM1 polypeptide, AP1—control polypeptide, AP0—negative control.

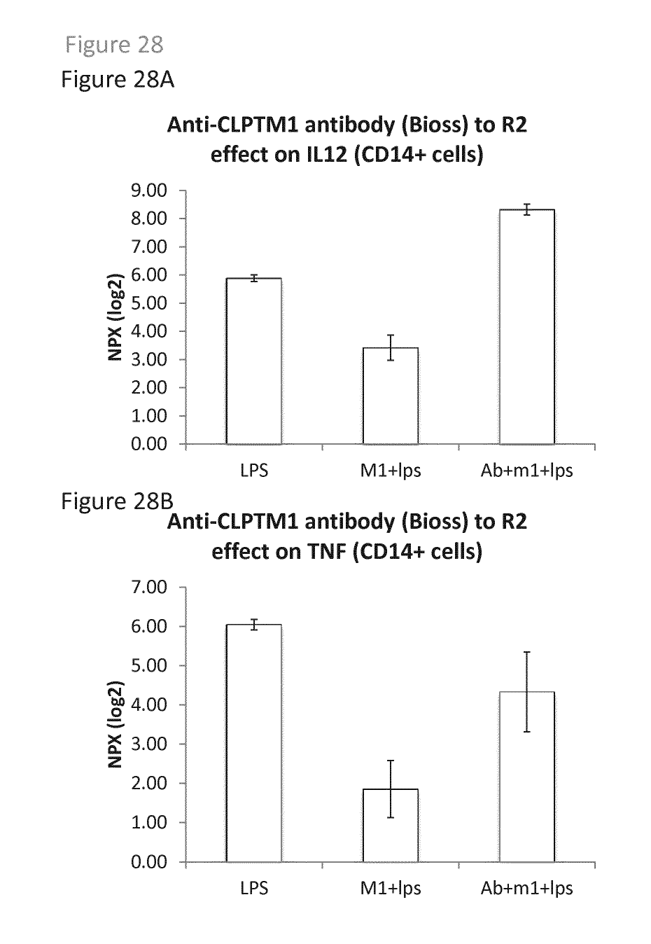

FIG. 28 shows the effect of an anti-CLPTM1 antibody on secretion of cytokines in LPS-stimulated CD14+ immune cells. Cytokines are secreted in the presence of LPS, and secretion is reduced in cells contacted with GDF15. Anti-CLPTM1 antibody restores the ability to secrete IL12 (FIG. 28A) and TNF (FIG. 28B).

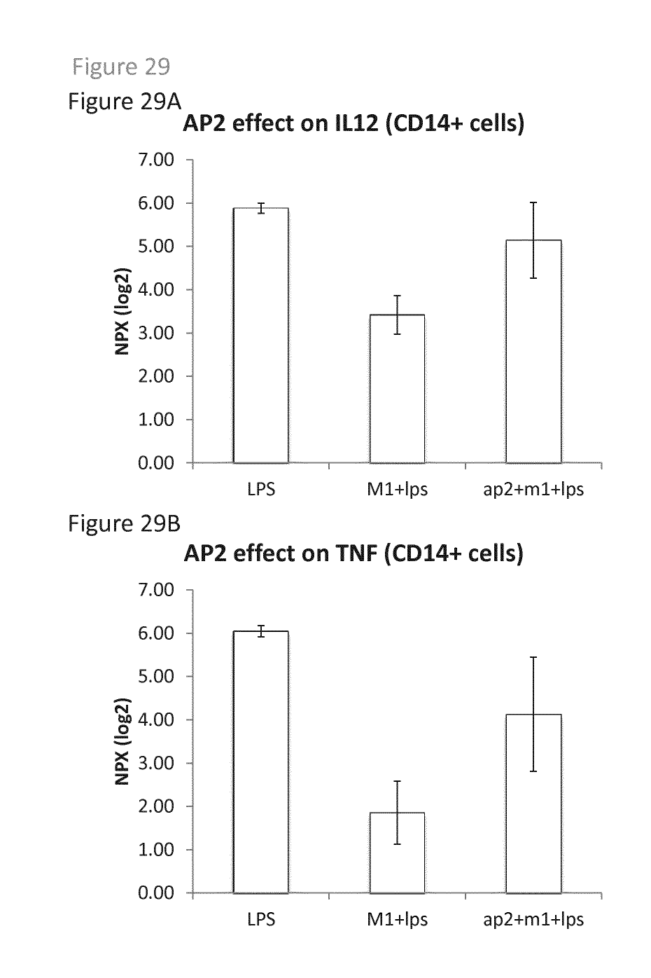

FIG. 29 shows the effect of an Fc fusion protein comprising a CLPTM1 polypeptide on secretion of cytokines in LPS-stimulated CD14+ immune cells. Cytokines are secreted in the presence of LPS, and secretion is reduced in cells contacted with GDF15. The Fc fusion protein comprising a CLPTM1 polypeptide restores the ability to secrete IL12 (FIG. 29A) and TNF (FIG. 29B).

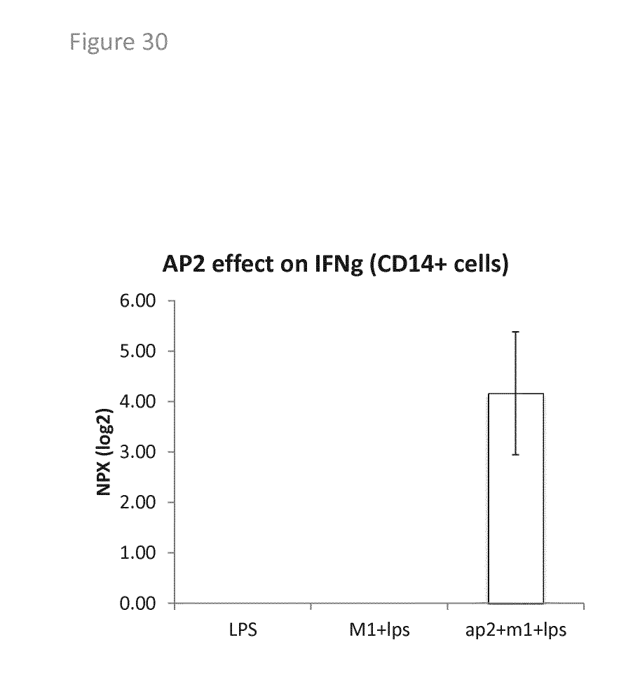

FIG. 30 shows the effect of an Fc fusion protein comprising a CLPTM1 polypeptide on secretion of IFNγ in LPS-stimulated CD14+ immune cells. No secretion is detected in either the presence or absence of LPS, but IFN γ is detected in cells contacted with the Fc fusion protein comprising a CLPTM1 polypeptide restores the ability to secrete IL12.

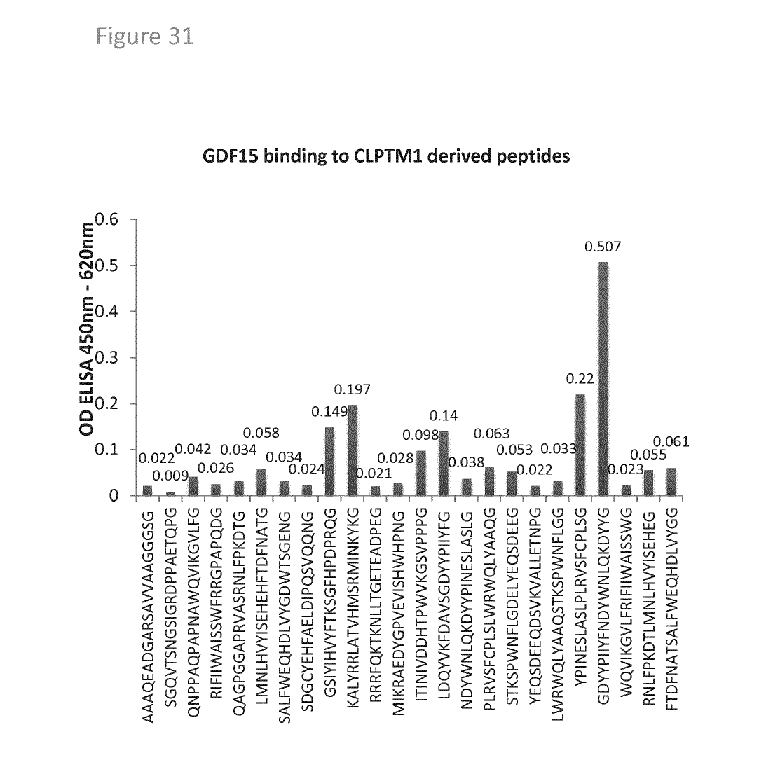

FIG. 31 shows the binding of GDF15 to CLPTM1 derived peptides. Polypeptides having amino acids as set forth in SEQ ID NOs: 250-273 were immobilised in streptavidin-coated well and binding of GDF15 to each peptide was measured.

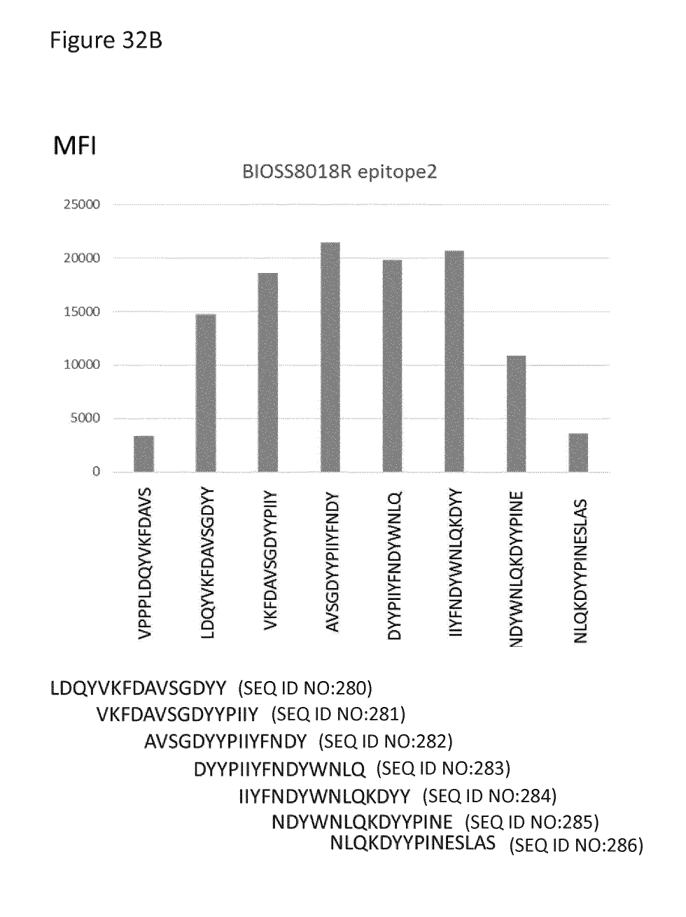

FIG. 32 shows the mapping of the epitopes of the polyclonal rabbit anti-CLPTM1 antibody (Bioss) on CLPTM1. Two separate epitopes were identified. Epitope 1 identification is shown in FIG. 33A, and Epitope 2 identification is shown in FIG. 33B.

### EXAMPLES

### Example 1—Tandem Affinity Purification-Mass Spectroscopy

We designed a protein-protein interaction capture assay using a set of different proteins with identical affinity tags, and included GDF15 in order to screen for interacting proteins. GDF15 was selected to be included in the panel due to important roles in human pathology, including various cancers, and a gap in the scientific literature on interacting proteins and receptor(s)

Design and Construction of Multiple Bait Proteins

Bioinformatic (SSPRO, RASMOL) analyses were done to exclude presence of hydrophobic amino acids in the baits and avoiding disruption of known secondary structure elements. Bait inserts were PCR amplified from plasmid DNA and inserted into a eukaryotic expression vectors that allowed Gentamycin selection.

The expression vectors were used to transfect cells and over-expressing stable cell lines were developed in parallel for a set of bait proteins, including GDF15.

After clonal selection and expansion of cells into range for sufficient amounts (10⁸ cells) to allow enough material for subsequent mass spectrometry analyses, the conditioned media was subjected to affinity purification against tags encoded in frame in the vector (and hence expressed as fusion to the bait proteins, including GDF15. Thus, the bait proteins were expressed in a eukaryotic host, increasing the probability of correct folding. Further, the “artificial” autocrine/paracrine loop constructed by this allowed bait ligand stimulation of the host cell to respond to the bait in various ways, such as release of exosomes containing receptors subsequently captured through co-purifying with the tagged bait.

In short, the tagged proteins such as GDF15 from separate cultures were captured using affinity matrix, washed extensively with buffers containing detergents and increasing salt concentration before elution from affinity matrix and subsequent processing for mass spectrometry analyses. Proteins that bound to GDF15 were digested, and subjected to MS analysis. Proteins were identified on the basis of the MS data.

Multiple runs were done, with various baits. The use of identical tags, only varying the specific bait allows identification of proteins uniquely co-purifying with a specific bait. A software script was written to ease the identification of unique hits for individual baits.

From these multiple runs, we identified QRFPR and CLPTM1 to uniquely co-purify with the GDF15 bait.

Development of Stable Cell Lines

Stable cell lines were established and expanded before harvest. The cells were washed with serum-free media, then allow to grow for 20 hrs in serum-free media.

On-Filter Tryptic Digestion of Proteins

Aliquots of 500 μL sample were digested on-filter essentially according to the protocol of Wisniewski et al (Wisniewski et al., 2009) using the 3 kDa filters (Pall Life Sciences, Ann Arbor, Mich., USA). The tryptic digestion was performed at 37° C. overnight in darkness. The samples were then centrifuged to collect the tryptic peptides in the filtrate while retaining undigested proteins and trypsin in the retentate. An additional volume of 100 μl of 50% ACN, 1% HAc was added and the filters were spun for 10 min and pooled with the first tryptic peptide filtrate. Finally, the collected filtrates were freeze-dried using a Speedvac system ISS110 (Thermo Scientific, Waltham, Mass., USA) and re-dissolved in 10 μl 0.1% TFA prior to nanoLC-MS/MS identification.

Nano LC-MS/MS for Protein Identification

The protein identification experiments were performed using a 7 T hybrid LTQ FT mass spectrometer (ThermoFisher Scientific, Bremen, Germany) fitted with a nano-electrospray ionization (ESI) ion source). On-line nanoLC separations were performed using an Agilent 1100 nanoflow system (Agilent Technologies, Waldbronn, Germany). The peptide separations were performed on in-house packed 15-cm fused silica emitters (75-μm inner diameter, 375-μm outer diameter). The emitters were packed with a methanol slurry of reversed-phase, fully end-capped Reprosil-Pur C₁₈-AQ 3 μm resin (Dr. Maisch GmbH, Ammerbuch-Entringen, Germany) using a pressurized packing device operated at 50-60 bars. The separations were performed at a flow of 200 nL/min with mobile phases A (water with 0.5% acetic acid) and B (89.5% acetonitrile, 10% water, and 0.5% acetic acid). A 100-min gradient from 2% B to 50% B followed by a washing step with 98% B for 5 min was used.

The bait proteins were produced from identical vector constructs, sharing affinity tags and linkers. We used these baits to screen for interacting proteins in conditioned media prepared from stable cells selected by G418, and identified interacting proteins via mass spectrometry (MS). In order to sort out hits that were specific for each bait we developed an in-house software; Data Drudger. From this analysis we identified tryptic peptides from QRFPR only when GDF15 was used as a bait. In our initial TAP-MS screen, a second protein was also found to co-purify with the GDF15 bait. This protein was identified as Cleft Lip and Palate transmembrane protein 1 (CLPTM1).

### Example 2—Purification of QRFPR-Containing Exosomes from Conditioned Medium

Exosome Isolation

Conditioned cell culture media were sequentially centrifuged for 5 min at 3,000 x g to pellet the cells and then at 10,000×g for 10 min to further remove cells and cell debris. The supernatant was then filtered through a 0.45 μm filter and then finally pelleted at 100,000×g for 2 h at 4° c. Pelleted exosomes were resuspended in PBS and protease inhibitor (complete mini, Roche) was added. The samples were then prepared for further analyses as described elsewhere.

Exosome Identification by Electron Microscopy

Pelleted exosomes were deposited on 20 mesh formvar/carbon coated copper grids (polysciences, Inc.) and then analysed by transmission electron microscopy (TEM) at 80 kV in a Technai G2 (FEI) (FIG. 1A).

Western Blot Identification of QRFPR Positive Exosomes

Material from the ultracentrifuged pellet was separated on a 12.5% SDS-PAGE gel (Biorad) at 150 v for 80 min, wet transferred to a Immobilon K membrane, blocked with 5% BSA for 1 h RT, and incubated with the corresponding QRFPR antibodies o/n at +4 with mild agitation. The membranes were washed with TBST, and the corresponding secondary antibodies, conjugated to horse radish peroxidase, were applied for 1 hr RT, the membrane washed with TBST and developed using an ECL detection reagent (FIG. 1B).

### Example 3—Detection of the GDF15-QRFPR at the Cell Surface

To investigate the presence of a complex between GDF15 and QRFPR on the cell surface, we analysed MCF-7 cells treated with Sulindac sulphide (a NSAID in order to increase low amounts of endogenous GDF15) using in situ PLA to detect the interaction between GDF15 and QRFPR. We compared the levels of proximity events between GDF15 and QRFPR in MCF-7 cells that were treated either with siRNA for QRFPR (FIG. 2B) or with a sequence-scrambled control siRNA (FIG. 2A). We detected a reduction of the levels of GDF15-QRFPR in QRFPR siRNA treated cells, thus validating the specificity of the antibody and the presence of the receptor in this cell line. The cells were serum starved prior to GDF15 treatment.

### Example 4—GDF15 Stimulation Reduces the Level of the QRFPR at the Cell Surface

Detection of ACTRIIB-QRFPR Proximity

Members of the TGF-β superfamily (to which GDF15 belongs) signal through a heterotetrameric receptor complex, consisting of two Type I and two Type II receptors. The Type II receptors bind the ligands and present them to the Type I receptors. We sought to identify the corresponding Type II receptor for GDF15.

There are only five known Type II receptors for the TGFb superfamily and it was initially hypothesized that ACTRIIB would fulfil some of the criteria for such a corresponding Type II receptor. The ACTRIIB is a type 2 receptor for GDF8, a member of the TGFb superfamily phylogenetically close to GDF15. Additionally, ACTRIIB has been suggested to be involved in fat metabolism. QRFPR has been described to be expressed in adipocytes.

MCF7 cells were stimulated with GDF15 (GDF15 from isolated mammalian source or recombinant derived from E. coli purchased from Abcam and Biovision). An assay was developed for monitoring the activity of GDF15 and to investigate the dynamics of receptor(s) such as QRFPR modulation by the ligand.

Serum-starved MCF7 cells were treated with GDF15, and an in situ PLA detection assay was developed to detect the interaction between ACTRIIB and QRFPR using antibodies from two different species, thus allowing the use of Duolink secondary detection reagents. The presence of the QRFPR-ACTRIIB complex was detected in the absence of GDF15 (see FIG. 3A, indicated by arrows). However, to our initial surprise, the signal obtained was reduced by GDF15 treatment by a brief stimuli (20 minutes), suggesting a dynamic regulation by GDF15 (see FIG. 3B). Interestingly, in experiments using MCF7 cells where cells were subjected to overnight serum starvation in order to avoid any confounding effects from serum, serum-starved cells were found to have increased QRFPR expression.

It is known from literature that GDF15 and QRFPR have opposite effects on two downstream targets, NPY and POMC. Thus, this suggests that GDF15 might interfere with the cell surface levels of QRFPR. If QRFPR was removed from the cell surface, the effect of its hydrophilic agonistic peptide ligands (26RFamide and QRFP) to regulate NPY and POMC would also be inhibited. The ability of GDF15 to regulate key targets central to mammalian metabolism, such as NPY and POMC, and its reported role in cachexia, might be explained through GDF15 inhibition of QRFPR.

Detection of EEA1-QRFPR Proximity

In order to investigate the role of GDF15 on QRFPR internalisation, a series of in situ PLA assays capable of monitoring endosome formation was developed.

Early endosome marker 1 (EEA1) is a marker for rapid endosome formation, and an assay was developed to detect the proximity of EEA1 and QRFPR. In the absence of GDF15, no signal was obtained (FIG. 3C). However, brief exposure of GDF15 (5 min) was found to be sufficient to increase the number of RCA products generated by proximity between EEA1 and QRFPR, as measured by an in situ PLA assay using antibodies towards EEA1 and QRFPR followed by Duolink secondary probes (see FIG. 3D, indicated by arrows).

We also observed that GDF15 induced the release of QRFPR positive exosomes, thus suggesting a bifurcation of the pathway, two ways by which GDF15 could reduce cell surface levels of QRFPR. We thus sought to develop assays incorporating markers known to have dual roles, both in endosome and exosome trafficking.

Detection of CD9-QRFPR Proximity

The tetraspanin CD9 has dual roles, both in endosome trafficking and in exosome secretion (Mazurov et al.). An in situ PLA assay was developed to detect the proximity of CD9 and QRFPR using antibodies against CD9 and QRFPR. In the absence of GDF15, only a low signal level is observed (FIGS. 4A and 4C). Stimulation with GDF15 for 30 minutes resulted in a higher level of signal for the CD9-QRFPR complex, indicating the formation of endosomes and exosomes (FIGS. 4B and 4D).

Detection of RAB11-QRFPR Proximity

The RAB11 protein is a marker for recycling endosomes back to the cell surface, and the RAB11 pathway is also involved in exosome assembly (Savina et al., 2005; Savina et al., 2002). RAB11 was thus thought to be a likely candidate to be involved in QRFPR reduction on the cell surface induced by GDF15

An in situ PLA assay was developed to detect the proximity of RAB11 and QRFPR. In the absence of GDF15, only a low signal is observed (FIGS. 5A and 5C). Stimulation with GDF15 resulted in the rapid detection of RAB11-QRFPR double positive endosomes in the cytoplasm (FIGS. 5B and 5D).

Inhibition of Proteasomal Degradation

In order to investigate if the endosome trafficking of QRFPR resulted in assembly and subsequent shedding of exosomes, or whether QRFPR was targeted for proteasomal degradation, an assay was developed in which cells were stimulated with GDF15 for a prolonged period of time.

It had been observed in previous experiments that GDF15 stimulation resulted in reduced levels of QRFPR at the cells surface. However, upon inhibition of the proteosome by the proteosome inhibitor MG132 (final concentration 10 μM), levels of QRFPR were increased compared to cells treated with equal amounts of GDF15, in the absence of MG132. FIG. 6A shows cells in the absence of GDF15, whereas FIG. 6B shows cells that have been stimulated with GDF15 overnight, and which have an undetectable level of QRFPR at the cell surface. FIG. 6C shows control cells that have been exposed to the proteasomal inhibitor MG132, and FIG. 6D shows cells that have been exposed to the proteasomal inhibitor MG132, and stimulated with GDF15 overnight.

The experiment demonstrates that GDF15 induces proteosome mediated degradation of QRFPR. In the absence of MG132, GDF15 treatment for 18 hrs dramatically reduced the levels of QRFPR (FIG. 6B). This effect is not seen in cells which are treated with MG132 (FIG. 6D).

Detection of RAB4-QRFPR Proximity

RAB11 positive endosomes have been reported to also incorporate RAB4, hence the existence of RAB4-RAB11 double positive endosomes. An assay was developed to detect the proximity of RAB4-QRFPR using antibodies against RAB4 and QRFPR. In the absence of GDF15, no signal was observed for RAB4-QRFPR (see FIG. 7A). The formation of RAB4-QRFPR double positive endosomes was detected upon GDF15 stimulation (see FIG. 7B).

RAB4-RAB11 double positive endosomes is a known endosome form, and we thus by two independent RAB markers (4 and 11) demonstrate that QRFPR positive endosomes are formed following GDF15 exposure of cells.

The formation of QRFPR positive endosomes may be used in assays to measure the effect of various GDF15 blocking agents, e.g. in Example 6.

### Example 5—In Vitro Interaction Assay

In order to further characterise the peptides which interact with GDF15, a series of GST-fusion proteins and biotinylated peptides was prepared. GDF15 was obtained from Biovision and/or Abcam.

Biotin Pull-Down of GDF15 Using Peptides Derived from QRFPR and CLPTM1

A series of peptides from the QRFPR and the CLPTM1 proteins was designed. In order to use the peptides both for immunoprecipitation experiments and cell culture based assays added an N-terminal biotin was added to each peptide, and the counter-ion changed to chloride.

A series of biotinylated peptides was used (FIG. 8A). Lane 1: Control non-biotinylated; Lane 2: Biotin-GEIKYDFLYEKEHICCLEEWTS (SEQ ID NO:10; Extracellular domain 3 from QRFPR); Lane 3: Biotin—GIEYSNFEKEYDDVTIK (SEQ ID NO:174; Extracellular domain 4 from QRFPR); Lane 4: Biotin-GALFWEQHDLVYGDWTS (SEQ ID NO:19; peptide from extracellular domain of CLPTM1); Lane 5: Biotin-peptide from unrelated protein (Ctrl) Two peptides were more successful than the others in precipitating GDF15 (FIG. 8A lanes 2 and 4). Combinations of the peptides were also used. Peptides were mixed in a 1:1 ratio, with same total amounts as in lanes 1-5, resulting in 50% of each peptide as compared to lanes 1-5. Lane 6: EC domain 3 & EC domain 4 from QRFPR; Lane 7: EC domain 4 from QRFPR & peptide from EC domain of CLPTM1; Lane 8: EC domain 3 from QRFPR & peptide from EC domain of CLPTM1; Lane 9: Mw markers; Lane 10: GDF15 (positive control).

Our previous experiment with RAB11-QRFPR endosomes and pull-downs suggested that the extracellular loop 4 also could participate in the interaction with GDF15. Thus it is possible that both the extracellular loop3 and loop 4 of QRFPR contacts GDF15.

Production of GST-Fusion QRFPR and CLPTM1 Fragments

We had with biotinylated peptides from QRFPR and CLPTM1 previously observed more retention of GDF15 with the motif derived from CLPTM1.

We then assembled the GDF15 binding motifs from QRFPR and CLPTM1 using synthetic hybridized oligonucleotides corresponding to the motifs from either extracellular domain 3, 4 (QRFPR) or CLPTM1. The oligonucleotides were inserted in a pGEX4T1 backbone, expressed in BL21 DE3pLys bacteria and purified. The correct sequences were confirmed by Sanger sequencing.

We then incubated the purified GST-fusion proteins with equal amounts of GDF15 and subjected the analytes to extensive washes including high salt buffers.

In line with the previous results using biotinylated peptides we observed a stronger affinity between GDF15 and the CLPTM1 derived motif.

In short, selected fragments of the receptors were constructed from synthetic oligomers (ultramers, IDT technologies), the flanking ends digested by restriction enzymes, and inserted in a pGEX 4T-1 vector (Pharmacia Biotech). The constructs were cloned in a DH5 a library efficiency bacteria (Invitrogen). The correct clones were identified by analytical restriction digest run on a 2% agarose gel, and validated by Sanger sequencing at the Uppsala sequencing centre.

The validated clones were used to transform BL21pLYs bacteria. Single clones were grown in 20 ml LB media supplemented with Ampicillin at a 100 μg/ml overnight and the following day expanded to 100 ml before induction with 0.1 mM IPTG for 3 hrs. Bacterial cells were pelleted by centrifugation, lysed by sonication and PBS with Triton x-100 (1%). The lysate was cleared by centrifugation, and the supernatant was incubated with glutathione beads (Pharmacia) overnight.

GST-Pulldowns of GDF15

Equal amounts of GST-fusion proteins were incubated with equal amounts of GDF15 in 500 μl of PBST in end-over-end rotation at 4° C. for 4 hrs and captured with Glutathione sepharose 4B beads (GE healthcare), then washed extensively with three different washing solutions (PBST, PBST with added 200 mM NaCl, and cell lysis buffer (0.5% deoxycholate, NP-40). Beads were transferred to a new Eppendorf tube during the washes in order to avoid remains of unspecific binding to tube wall before boiling in SDS.

Final Wash in PBS

The material was denatured by boiling in SDS loading dye supplemented with DDT for 5 min, then run on a 12% acrylamide gel (120 V for 70 min) and analysed by ECL and CCD camera (Biorad).

Lane 3: GST—EIKYDFLYEKEHICCLEEWTS (GST-fusion with SEQ ID NO:7; EC domain 3 of QRFPR); Lane 4: GST—ALFWEQHDLVYGDWTS (SEQ ID NO:18; peptide from EC domain of CLPTM1). Lane 7: GST control (no fusion). Increased binding was observed for peptides corresponding to extracellular domain 3 of QRFPR and a portion of the extracellular domain of CLPTM1 (FIG. 8B, lanes 3 and 4).

### Example 6—Blocking the GDF15-QRFPR Interaction

Blocking Internalisation Using Peptides in MCF7 and U2OS Cells

We choose to use this assay as a way to investigate if the activity of GDF15 could be inhibited by various agents. A receptor fragment from extracellular loop 3 of QRFPR substantially reduced the ability of GDF15 to induce RAB11-QRFPR double positive endosomes (see FIG. 9). By contrast, a control fragment from an unrelated protein does not effect the inhibition of increased RAB11-QRFPR double positive endosomes. GDF15-induced formation of RAB11/QRFPR endosomes is inhibited by QRFPR peptides (FIG. 9A-D). Equal amounts of a mastermix of GDF15 was aliquoted and pre-incubated in PBS with the various receptor fragment before addition to cells. Sequentially, the cells were treated with either PBS, GDF15 or GDF15/QRFPR-derived peptide mixes. The level of QRFPR at the cell surface was reduced by GDF15 in U2OS cells, but restored to normal levels by a QRFPR peptide (FIG. 9E)

Blocking Internalisation Using Peptides Whilst not Affection P518

FIGS. 10A-10C confirm that GDF15 reduces the level of QRFPR at the cell surface, and that this reduction may be reversed by a peptide derived from extracellular loop 3 (extracellular domain 4) of QRFPR.

The effect of the QRFPR agonist p518 was measured. In short, p518/QRFP-26 was purchased from Phoenix Peptides, CA and processed according to the manufacturer's instructions. Cells were serum-starved prior to P518 stimulation overnight.

P518 was mixed with either PBS or QRFPR receptor fragment dissolved in PBS prior to stimulation of cells.

We demonstrated that p518 alone was sufficient to increase levels of ACTRIIB, and interestingly, that the presence of QRFPR fragment did not decrease levels of P518 induced ACTRIIB levels. On the contrary, we noticed a slight increase of ACTRIIB when cells were treated with both p518 and QRFPR receptor fragment, compared to mono-treatment with p518 (see FIG. 10 D-F).

Thus we observe that the QRFPR receptor fragment does not inhibit the agonist of QRFPR, P518, however, it has the capacity to reduce the GDF15 effects on QRFPR levels demonstrated in a separate assay for QRFPR levels (FIG. 10 A-C).

By using these two assays it is therefore possible to detect and develop selective inhibitors (such as the herein used QRFPR fragments or QRFPR antibodies directed at a epitope that does not interfere with p518) that inhibit GDF15, yet spare the agonist (p518)

Antibodies Preventing Rab4 Endosomes

In short, the cells were serum starved overnight then pre-incubated with blocking antibodies towards extracellular parts of QRFPR for 2 hrs prior to GDF15 treatment (1 μg/ml) for 30 minutes. The levels of formed RAB4-QRFPR complexes were measured by visualization of formed RCA products using the Duolink is PLA system (see FIG. 11). Blocking antibodies were found to inhibit the formation of RAB4-QRFPR endosomes. The blocking antibodies used were demonstrated on Pep-star array to overlap with the GDF15 binding site on QRFPR.

This assay was useful in order to prove that antibodies towards QRFPR were able to inhibit GDF15 mediated endosome formation.

The ACTRIIB was initially chosen in order to investigate the possibility of whether it might be a possible type II receptor for GDF15. This was based on the fact that GDF15 is closely related to GDF8, which interacts with ACTRIIB. It was found that GDF15 decreased the extent to which QRFPR and ACTRIIB interacted (as identified by a reduction in the number of proximity events), by at least two mechanisms: endosome mediated internalization (e.g. via RAB4 and 11 endosomes) and by exosome shedding of QRFPR.

Surprisingly, it was found that the two known agonists for QRFPR (p518/26RFa(26-mer) and QRFP(43-mer) act in opposite manner to each other to regulate the levels of ACTRIIB. It was identified that this might provide a novel assay useful to enable the development of selective inhibitors for the GDF15 binding site of QRFPR, whilst sparing the binding of the agonistic ligands.

Preferably, such a binding agent, (e.g. an antibody) may block access of GDF15 to QRFPR by directly binding to an epitope that overlaps the GDF15 binding site, or indirectly by binding to adjacent epitopes and blocking binding by e.g. steric hindrance, thus inhibiting access of GDF15 to QRFPR.

Preferably, such binding agents do not interfere with the agonistic function of the receptor mediated by binding to the endogenous ligands. This ACTRIIB assay provided herein thus allows it to be determined whether such a binder interferes with agonistic binding of ligands.

We were able to use the assay to determine that the peptide used to inhibit GDF15 did not interfere with QRFP. Thus selective tools to modulate the function of QRFPR agonists and antagonists can be detected by using both the ACTRIIB assay and the RAB11-QRFPR endosome assay.

### Example 7—Peptide Screen

In order to determine the key amino acids and a minimal epitope present in the third extracellular domain of QRFPR (extracellular loop 2) we designed a Pepstar peptide array, purchased from JPT peptides. In short the array included complete coverage of the extracellular loop2 of QRFPR, divided into 15-mers. Peptides of this length (15-mers) were selected based on recommended length by the manufacturer, based on factors such as efficiency of synthesis, purity and yield by FMOC chemistry. The 15-mers were either varied according to amino acid walk, single or double alanine scan. Each set was analysed in triplicates. GDF15 was diluted into PBS-T (0.05% Tween-20) and incubated under mild agitation on an orbital shaker overnight at +4 C. Washes was done under agitation on a Sky line orbital shaker DOS-10L, ELMI.

Primary antibodies against GDF15 were added to wells with prior GDF15 incubation and to adjacent control wells where GDF15 had been omitted (only PBS-T) and incubated for 1 hr RT under agitation on orbital shaker. After repeated washes fluorophore-conjugated secondary antibodies were added, and after final washes the array was analysed on a G2502 Microarray scanner, Agilent Technologies. Fluorescence values from technical controls and control wells (no GDF15) were subtracted and resulting values in triplicate was analysed in Excel and Prism.

From the experiment we were able to determine a minimal set of key amino acids important for the interaction between GDF15 and QRFPR. Amino acid walk and alanine scan revealed that the stretch FLYEKEHIC (SEQ ID NO:11) was essential for GDF15 binding. (FIG. 12) Additional stretches were also capable of retention of GDF15 as has been listed in this application.

Interestingly, GPRs such as QRFPR have a conserved cysteine residue in the extracellular loop2(ECL2), that forms a disulphide bridge with cysteines in extracellular loop1.

Binding of agonist and antagonist ligands induce different lid conformations in ECL2 around the conserved disulphide bond These have been suggested to produce different functional states of the receptors. We demonstrated in our experiment that GDF15 has the capacity to interact precisely at this site, forming an elegant regulatory mechanism. By interacting exactly there, GDF15 antagonizes the orixogenic function of QRFPR, thereby regulating the levels of key metabolism regulating hormones such as NPY and POMC. We suggest that this is a key mechanism (and that the FLYEKEHICC (SEQ ID NO:78) is a core binding site), of immense importance to understand cachexia as well as disturbances of bone metabolism induced by elevated levels of GDF15 in numerous cancer forms, as well as other human disorders. We thus suggest that an agent such as an antibody binding to ECL2 (ECD3) of QRFPR, or adjacent to and by steric hindrance blocking the ability of GDF15 to bind QRFPR has the potential to be a therapeutic agent for disorders where GDF15 is elevated.

### Example 8—Epitope Mapping

In order to determine the epitopes of antibodies used in cell culture experiment we treated parallel wells with the various receptor binding antibodies. Experiment and Analyses as for the GDF15 analysis in FIG. 14, except that the antibodies was incubated for 1 hr in RT. From we were able to identify antibodies with overlap to the epitope used by GDF15 (FIG. 13). Amino acid walk demonstrates that the QQLEIK (SEQ ID NO:335), especially K, is necessary for N-15 goat ab to bind. The N-15 ab further needs the FLYEK (SEQ ID NO:210) as revealed by ala scan (marked in red). Thus the ab partly overlaps the site occupied by the ligand.

TABLE 2
array screening peptide sequences

| Polypeptide sequence | SEQ ID NO: |
|---|---|
| QQLEIKYDFLYEKEH | 176 |
| QLEIKYDFLYEKEHI | 39 |
| LEIKYDFLYEKEHIC | 40 |
| EIKYDFLYEKEHICC | 41 |
| IKYDFLYEKEHICCL | 42 |
| KYDFLYEKEHICCLE | 43 |
| YDFLYEKEHICCLEE | 44 |
| DFLYEKEHICCLEEW | 45 |
| FLYEKEHICCLEEWT | 73 |
| LYEKEHICCLEEWTS | 324 |
| YEKEHICCLEEWTSP | 325 |
| EKEHICCLEEWTSPV | 326 |
| KEHICCLEEWTSPVH | 327 |
| EHICCLEEWTSPVHQ | 328 |
| HICCLEEWTSPVHQK | 329 |

### Example 9—GDF15-CLPTM1

GDF15 has been reported in the literature to mediate downstream signalling resembling both the canonical (SMAD) pathway mediated by ALK5(TGFBRI) kinase domain, and non-canonical TGFβ receptor signalling, such as phosphorylation of GSK3b. In order to investigate if CLPTM1 was involved in this we developed a set of in situ PLA assays to determine the dynamics between CLPTM1 and the TGFβ receptors and components downstream of the TGFβ receptors. NK-92 cells were treated with GDF15 for 40 min to detect phosphorylated SMAD proteins such as SMAD2 in the nucleus. We failed to detect c-terminal phosphorylated SMAD 2 upon GDF15 stimulation in NK-92 cells. From previous experiments with TGFB1 stimulation we know that the assay (pSMAD2) was functional.

Surprisingly, we detected an induced proximity between CLPTM1 and TGFBRI(ALK5) upon stimulation with GDF15 as demonstrated in FIG. 14.

It was not known whether TGFBRI localised with CLPTM1 independently from the corresponding Type II receptor TGFBRII, or whether the TGFBRI-TGFBRII heterocomplex associated with CLPTM1 upon GDF15 stimulation. In order to determine this, a second in situ PLA assay was developed, using antibodies towards TGFBRII in concert with CLPTM1 antibodies from another species. We then discovered that GDF15 treatment also induced CLPTM1 proximity to TGFBRII. (FIG. 15)

Current hypotheses in the literature regarding the stoichiometry and dynamics of the heterotetrameric TGFBRI-TGFBRII complex suggests two alternative forms on the cell surface; 1) association of two TGFBRI and two TGFBRII receptors upon ligand stimulation (e.g. (TGFB1)), or 2) pre-existing heterotetrameric complexes (i.e. before ligand binding).

Current hypotheses in the literature suggest different preferred downstream signalling pathways, where the former (ligand induced heterotetramer) more supports the canonical (SMAD) pathway and the latter (where the ligand is presented and binding pre-existing heterotetramers) supports non-canonical such as phosphorylation of non-SMAD substrates.

Based upon the absence of SMAD2 phosphorylation, and reports in the literature on non-canonical downstream signalling initiated by GDF15 and the discovery by us that both the receptors are associated with CLPTM1 upon GDF15 stimulation we hypothesised that CLPTM1 participates in presenting GDF15 to a pre-existing TGFBRI-TGFBRII heterotetrameric complex.

In order to test this theory, an assay to test the effect of GDF 15 binding to CLPTM1 using CLPTM1 binding agents was developed (see Example 10).

### Example 10—Blocking GDF15-CLPTM1

The effect of antibodies binding CLPTM1 on inhibiting GDF15 induced downstream signalling through the TGFB heterotetrameric complex was assessed by detecting phosphorylated GSK3b-pGSK3b(9/21) in an in situ PLA assay. Phosphorylation of GSK3b is known to cause immunosuppressive effects i.e. suppress immune cell function.

In order to validate that CLPTM1 is involved in GDF15 and to demonstrate for the first time that an blocking agent such as an antibody could reduce GDF15 induced downstream signalling we treated NK-92 cells with GDF15 in the presence of control antibody or CLPTM1 antibody (see FIG. 16). We demonstrate that in the presence of control antibody, GDF15 induces 9/21 phosphorylation of GSK3b (in accordance with literature). However, the CLPTM1 binding antibody reduces GDF15 induced phosphorylation of GSK3b. Without wishing to be bound by theory, this would suggest that an antibody binding to CLPTM1 might be able to inhibit the immunosuppressive role of GDF15 through GSK3b phosphorylation.

### Example 11—Peptide Screen

We investigated the binding of GDF15 to CLPTM1 as described in Example 7 for QRFPR, using a peptide derived from the extracellular domain of CLPTM1. From BLAST homology between the two receptors we identify two potential sites in the extracellular domain of CLPTM1 with corresponding amino acid sequence extracellular loop 2 in QRFPR (YISEHEH (SEQ ID NO:15) and LFWEQH (SEQ ID NO:16)).

The epitope of CLPTM1 may potentially comprise a larger stretch then the 15-mer peptides which were used in the array, and both the biotin-peptide and GST-fusion experiments with a longer peptide stretch and increased avidity (tetramer and dimer) (see Example 5) provides better GDF15 binding then a linear 15-mer.

From the array experiment (FIG. 17), several peptides from CLPTM1 bind GDF15 and are listed in this application.

TABLE 3
CLPTM1 screening peptides

| Polypeptide | SEQ ID NO: |
|---|---|
| ISEHEHFTDFNATSA | 330 |
| SEHEHFTDFNATSAL | 320 |
| EHEHFTDFNATSALF | 331 |
| HEHFTDFNATSALFW | 332 |
| EHFTDFNATSALFWE | 333 |
| HFTDFNATSALFWEQ | 334 |
| FTDFNATSALFWEQH | 149 |
| TDFNATSALFWEQHD | 135 |
| DFNATSALFWEQHDL | 171 |
| FNATSALFWEQHDLV | 170 |
| NATSALFWEQHDLVY | 169 |
| ATSALFWEQHDLVYG | 168 |
| TSALFWEQHDLVYGD | 167 |
| SALFWEQHDLVYGDW | 166 |
| ALFWEQHDLVYGDWT | 142 |

The identification of relatively short peptides, and particularly the binding motifs identified herein, provide start points for therapeutic products which may be used to block the interaction between GDF15 and its receptors. Whilst not wishing to be limited to particular embodiments, potential GDF15 decoy peptides might be provided as cyclic peptides or Fc-fusions for therapeutic use.

### Example 12—Epitope Mapping

We determined that the antibodies towards CLPTM1 used in the present studies recognised an epitope that overlaps with the GDF15 binding site, as described in Example 8 (see FIG. 18).

TABLE 4
CLPTM1 antibody epitope mapping

| Polypeptide | SEQ ID NO: |
|---|---|
| YISEHEHFTDFNATS | 102 |
| ISEHEHFTDFNATSA | 330 |
| SEHEHFTDFNATSAL | 320 |
| EHEHFTDFNATSALF | 331 |
| HEHFTDFNATSALFW | 332 |
| EHFTDFNATSALFWE | 333 |
| HFTDFNATSALFWEQ | 334 |
| FTDFNATSALFWEQH | 149 |
| TDFNATSALFWEQHD | 135 |
| DFNATSALFWEQHDL | 171 |
| FNATSALFWEQHDLV | 170 |
| NATSALFWEQHDLVY | 169 |
| ATSALFWEQHDLVYG | 168 |
| TSALFWEQHDLVYGD | 167 |
| SALFWEQHDLVYGDW | 166 |
| ALFWEQHDLVYGDWT | 142 |
| LFWEQHDLVYGDWTS | 125 |

We propose that antibodies binding outside the GDF15 binding site on CLPTM1 may also inhibit GDF15 binding to CLPTM1 by steric hindrance, and thus may also be useful in applications such as immune-oncology in which it is desirable to inhibit GDF15 mediated crosstalk with the TGFb receptor complex associated with CLPTM1 binding. The immunosuppressive effects of GDF15 may thereby be blocked.

### Example 13—Expression of CLPTM1 on Immune Cells

In order to investigate the extent to which CLPTM1 is expressed on human immune cells, we FACS-sorted various immune cell types from healthy donor PBMC, and detected CLPTM1 expression in CD4+ and CD8+T-lymphocytes and CD45+ CD3+ Non-T-lymphocytes (FIG. 19), and in CD11c+ cells (dendritic), and CD14+ cells (monocytes/macrophage) (FIG. 20).

In CD4 and CD8 T-lymphocytes we detected a subset of cells expressing high levels of CLPTM1. In CD14 positive cells we detected an overall high amount of CLPTM1. (FIG. 20).

The access to healthy donor PBMC and gating strategy on FACS used in selection of immune cell types was done through clinical immunology unit, KS, Stockholm and followed established protocols.

The extent to which CLPTM1 was expressed on cells of the immune system suggests that it may have wide-reaching effects in the modulation of the immune system, and may be able to regulate the immune response in a wide range of different immune response pathways.

### Example 14—Inhibition of Down-Regulation of NKG2D by GDF15

Members of the TGFb superfamily, such as TGFb1 and GDF15 has been reported to suppress the cytolytic function of immune cells expressing NKG2D, such as NK and T-cells.

NKG2D is upstream of key effector proteins such as perforin and Granzyme, and TGFb1 and GDF15 have been reported to suppress levels of such cytolytic proteins.

Based upon literature, the expression of CLPTM1 in immune cells such as CD8+T-lymphocytes and NK-92 cells, and the GDF15 induced proximity of CLPTM1 to TGFBRI-TGFBRII (through which TGFB1 signals), we investigated whether blockade of CLPTM1 by antibodies could affect the levels of the immune effector NKG2D in NK-92 cells that had been subjected to extended stimulation with GDF15. FIG. 21A shows that overnight stimulation with GDF15 results in low levels of detectable NKG2D in NK-92 cells. The decrease in NKG2D was reversed in NK-92 cells that had been pre-treated with a binding agent capable of binding to CLPTM1 (an antibody) prior to GDF15 stimulation (FIG. 21B). CLPTM1 antibodies can therefore increase the levels of NKG2D in NK-92 cells. We propose that this may increase their cytotoxicity.

### Example 15—Cancer Indications

Detection of CLPTM1-GDF15 in Immune Cells in a Tumour Microenvironment

We investigated the presence of the two receptors in various human cancers using commercially available tissue micro arrays, purchased from US Biomax.

The presence of infiltrative cells positive for the CLPTM1-GDF15 PLA interaction in a set of human cancers, exemplified here in FIG. 22, which shows metastases from Squamous lung cell carcinoma. By counterstaining with FITC labelled CD3 we were able to detect double positive cells. However, we also detected CD3+ CLPTM1-GDF15 negative cells, possibly representing a population of infiltrative cells that are negative for CLPTM1 as observed in our FACS data.

Additionally, we also detected CD3− cells that showed the CLPTM1-GDF15 complex, which might represent other immune cells such as CD14+ cells (e.g. monocytes). The complexity of a tissue such as a tumour metastasis with different infiltrating immune cell types warrants further studies with other markers, such as CD14.

The presence of tumour infiltrating CD3+ cells which also demonstrate detectable CLPTM1 bound to GDF15 in the GDF15-rich tumour micromilieu suggests a potential for CLPTM1 binding agents (such as antibodies), that inhibit GDF15 access to CLPTM1. Such a product may be useful in reducing GDF15 induced immune-suppression.

Detection of QRFPR-GDF15 in Bone Cancers

For QRFPR, we detected QRFPR-GDF15 in the borders of prostate cancer micrometastasis in bone (see FIGS. 23, 24A and 24B) Low levels of signal was detectable in primary prostate cancer (see FIG. 24C), which compares with the high level of signal detectable in prostate cancer metastases to bone.

This is of particular interest, as GDF15 is used as a biomarker for bone metastases from prostate. Additionally, QRFPR is known to be expressed in osteoblast and is involved in bone metabolism. Thus a QRFPR binding agent (such as an antibody) or a polypeptide that is capable of blocking GDF15 access to QRFPR may be useful in reducing GDF15 induced dysregulation of osteoblast metabolism in prostate cancer.

Such an antibody would be of particular clinical value in such disorders, particularly if used to treat aspects of dysregulation of bone metabolism separately from current treatment regimes, such as bisphosphonates, which act on osteoclasts.

Expression of QRFPR was also found in primary bone cancers: Ewing sarcoma (FIG. 25) and osteosarcoma (FIG. 24D). The latter expression is in line with our observation of QRFPR staining in U2OS osteosarcoma cells, and GDF15 regulation of QRFPR in U2OS cells. Thus, a QRFPR binding agent or a polypeptide which blocks the interaction between GDF15 and QRFPR may be of clinical value in such disorders, particularly if used to treat aspects of dysregulation of bone metabolism separately from current treatment regimes, such as bisphosphonates, which act on osteoclasts.

Finally, the QRFPR-GDF15 complex was detected in Myeloma (FIG. 26), representing a possible further indication for such a therapeutic agent.

### Example 16—Pull Down Interaction of QRFPR and CLPTM1 Peptides with GDF15

20 μg Fc fusion protein constructs were incubated with 0.5 μg GDF15 and protein-G bead with mixing for 6 hrs in +4 C in PBST+1% BSAs. Wash 6 times: 3 times PBST, change tubes, 2 washes PBST+220 mM NaCl. Last wash PBS. The beads were collected using a table-top centrifuge set at 500 g for 5 minutes between washes. Detection The material was loaded on a 4-12% gradient gel, run at 100 volt, transferred to PVDF membrane using a Turbo Tank blot system, blocked by PBS with 3% BSA 1 hr RT, and incubated with RD systems a monoclonal anti-GDF15 antibody overnight. A secondary antibody: mouse HRP 1:2000 was used for detection. The size of band was estimated by comparing with a seeblue2 protein ladder marker run in an adjacent lane.

Fc-fusion proteins incorporating sequences from mouse were designed:

The (GGGGS)×3 was used as a flexible linker (SEQ ID NO:242).

AP5 (QRFPR peptide)
(SEQ ID NO: 240)
Fc mouse IgG1-GGGGSGGGGSGGGGSQRLEIKYDFLYEKEH
AP1 (control protein)
(SEQ ID NO: 323)
AAAQEADGARSAVVAAGGGSSGQVTSNGSIGRDPPAETQPQNPPAQPAP
NAGGGGSGGGGSGGGGS-Fc mouse IgG1
AP2 (CLPTM1 peptide)
(SEQ ID NO: 241
Fc mouse IgG1-
GGGGSGGGGSGGGGSYISEHEHFTDFNATSALFWEQHDLVYGDWTS

AP0

Negative Control

The fragments from the receptors were selected based on previous results including array data indicating direct interaction with GDF15. In the array, the interaction is between a peptide monomer and the ligand. A pull down experiment with stringent washing conditions was performed and GDF15 detected, as shown in FIG. 27.

Discussion: After extensive washes, we noticed that AP5 construct comprising a peptide from QRFPR and AP2 construct comprising a peptide form CLPTM1 were capable of pulling down GDF15

Conclusion: We have shown that GDF15 has an affinity for both receptors, and that both constructs could be envisioned for therapeutic use as a ligand trap in disorders where elevated GDF15 has a pathological role. The data agrees with the array results, where for example the polypeptide in AP1 did not interact with GDF15 but the AP2 sequence did.

Without wishing to be bound by theory, the full length CLPTM1 receptor may have the potential to offer a higher affinity.

### Example 17—Antibodies and Peptides Blocking GDF15

The effect of GDF15 on immune cells was established, and it was found that this effect could be blocked by an antibody targeted to CLPTM1, and by an Fc fusion construct comprising a portion of the extracellular domain of CLPTM1.

CD14+ cells where isolated and cultured in the presence or absence of GDF15 in a LPS stimulated culture. GDF15 demonstrated an immune suppressive effect, as the LPS response secretion of TNF and IL12 was reduced in cells incubated with GDF15. A polyclonal rabbit antibody (bs-8018R supplied by Bioss Antibodies) reduced this immune suppressive effect as shown by the increase in secretion of both cytokines in FIG. 28.

A similar reversal of the immunosuppressive effects of GDF15 was also demonstrated by a polypeptide derived from the extracellular domain of CLPTM1 (SEQ ID NO:17). The polypeptide was provided as an Fc fusion comprising an N-terminal linker sequence, and demonstrated similar effects on TNF and IL12, as shown by the increase in secretion of both cytokines in FIG. 29. The sequence of the peptide including its linker sequence is shown in SEQ ID NO:241.

Incubation of CD14+ cells stimulated with LPS in the presence of the Fc fusion protein comprising the CLPTM1 polypeptide also demonstrated secretion of the pro-inflammatory protein IFNγ, as shown in FIG. 30.

Materials and Methods

Isolation of Peripheral Blood Mononuclear Cells (PBMCs)

Buffy coat cell suspension or heparinized blood samples were diluted with sterile, room temperature PBS. Peripheral blood samples are diluted 1:1, whereas buffy coats are diluted to a final volume of 120 ml (in a standard buffy coat this will result in slightly greater dilution than 1:1). The diluted cell suspension was layered onto Ficoll Paque PLUS. For one buffy 4×15 ml aliquots are used (30 ml of diluted cell suspension/tube). Cells were centrifuged at 400×g for 30 min at room temperature and transferred to a new 50 ml tube. Cells were washed once and resuspended in 20 ml PBS.

Magnetic Sorting of CD14+ Cell Fraction

Mononuclear cell suspension was pelleted by centrifugation. Cells were resuspended in Stemcell buffer at a concentration of 10⁸ cells/ml and transferred to a round bottom 14 ml tube. EasySep Positive Selection cocktail at was added at 100 μl/ml cells, mixed by gentle pipetting, and incubated at room temperature for 15 min. 50 μl EasySep Magnetic Nanoparticles per 1 ml cells were added and mixed, and incubated at room temperature for 10 min. Stemcell recommended medium was added to bring the cell suspension to a total volume of 5 ml and mixed by gently pipetting. The tube (without cap) was placed into the magnet and set aside for 5 min. After 5 minutes the supernatant was discarded. CD14+ labelled cells will remain inside the tube. The tube was removed from the magnet and add 5 ml Stemcell recommended medium was added and mixed by gently pipetting. The tube (without cap) was placed into the magnet and set aside for 5 min. The was step was repeated before the cells were resuspended in 5 ml complete RPMI medium. 50 μl of the CD14+ cell suspension was removed and counted to determine total cell number after dilution in Trypan blue solution and mounting on Fast Read 102 Counting Slides.

Culture Setup and Stimulation

Day 0

Adjust CD14+ cell suspension volume to a concentration of 0.833×106 cells/ml in complete RPMI medium. 300 μl of cell suspension is added to wells in a 48 well plate well (0.25×10⁶ cells per well), and allowed to adhere for 90 min in an incubator at 37° C. Non-adherent cells were removed by removal of the supernatant after gently pipetting the medium. Cells were washed once with 300 μl pre-warmed complete RPMI medium and the medium aspirated off. 300 μl pre-warmed complete RPMI medium was added to each well and the cells were incubate overnight (20-24 h) in a cell incubator at 37° C.

Day 1

A 17× stock solution of the desired GDF15 modulatory substances was diluted in complete RPMI. (20 μl of substance/well assayed). 20 μl of modulatory substance or medium/carrier (negative control) was added to each well and mixed by gentle pipetting. Cells were incubated for 30 min in a cell incubator at 37° C. A 17×GDF15 solution was prepared by diluting a 100 μg/ml stock solution to a concentration of 8.5 μg/ml (final concentration in culture 0.5 μg/ml) in complete RPMI. 20 μl of diluted GDF15 was added per well, and incubated overnight (18-20 h) in a cell incubator at 37° C.

Day 2

A 18×LPS solution was prepared by diluting the LPS stock solution to a concentration of 18 ng/ml (final concentration in culture 1 ng/ml) in complete RPMI. 20 μl of LPS or medium (negative control) was added to each well and mixed by gentle pipetting. Cells were incubated for 4 h in a cell incubator at 37° C. 200 μl of supernatant from each well was transferred to a V-bottom 96-well plate. Samples were centrifuged to remove any cells or debris. Supernatants were transferred to plastic 96-well PCR plate. Proceed to TNF-α analysis immediately or cover wells with adhesive plastic cover and store at −80° C. until further analysis.

Proseek Assay

Secreted proteins from cell cultures where quantified using multiplexed proximity extension assay, Proseek (Assarsson E et al PlosOne 2014) by Olink Proteomics AB (Uppsala Sweden). Data is presented as Normalized Protein Expression (NPX log 2). The Oncology v2 92-plex panel was run. Data from important pro-inflammatory markers IL12, TNF, and IFNg are presented.

### Example 18—GDF15 Binding to CLPTM1 Derived Peptides

Materials and Methods

Biotinylated synthetic peptide fragments from CLPTM1 (JPT peptides, Germany) were immobilized in a streptavidin 96 well plate (#15500 Pierce) at 1 μM in PBS over night at +4° C. Plate was washed 4 times in 300 μl PBS with 0.05% Tween-20 (wash buffer). The plate was blocked with PBS 1% BSA and 0.05% Tween-20 (blocking buffer) for 1.5 hours at room temperature then washed 4 times in wash buffer. GDF15 ligand (Abcam) was added in blocking buffer at 100 ng/ml at incubated at room temperature for 2 hours. The plate was then washed 8 times 300 μl in wash buffer with 2× NaCl. A GDF15 antibody as added at 1 ug/ml (RnD systems goat polyclonal) and incubated in block buffer for 1 hour at room temperature followed by washing with 4×300 μl wash buffer. HAF017 anti-goat-HRP antibody was added and incubated for 1 hour in blocking buffer followed by a wash. TMB substrate was added and stopped after 15 minutes with H₂SO₄. OD 450-620 was measured in an ELISA reader.

Polypeptides having sequences set forth in SEQ ID NOs:250-273 were assessed for binding to GDF15 (corresponding to the polypeptides indicated from left to right in FIG. 31). These proteins were synthesised with a C-terminal glycine residue not present in the corresponding sequences from CLPTM1. The signal measured for each polypeptide is shown in FIG. 31. A cut-off of approximately greater than or equal to 0.1 OD in the ELISA readout was identified as indicating binding of the GDF15 to an immobilised polypeptide (i.e. including the 0.098 OD value for GDF15 binding to the polypeptide having the sequence set forth in SEQ ID NO:258). The following peptides showed binding to GDF15 (Residue numbers relative to SEQ ID NO:2):

(162-181)
(SEQ ID NO: 258)
GSIYIHVYFTKSGFHPDPRQG
(182-201)
(SEQ ID NO: 259)
KALYRRLATVHMSRMINKYKG
(242-260)
(SEQ ID NO: 262)
ITINIVDDHTPWVKGSVPPPG
(262-280)
(SEQ ID NO: 263)
LDQYVKFDAVSGDYYPIIYFG
(292-311)
(SEQ ID NO: 269)
YPINESLASLPLRVSFCPLSG
(273-292)
SEQ ID NO: 270)
GDYYPIIYFNDYWNLQKDYYG

The corresponding polypeptides to SEQ ID NO:258, 259, 262, 263, 269 and 270 lacking the C-terminal glycine residue are 297, 298, 301, 302, 308 and 309, respectively. Such polypeptides or parts thereof, or polypeptides comprising these sequences or parts thereof, or closely related sequences, represent one group of preferred polypeptides according to the present invention

### Example 19—Epitope Mapping of ‘Bioss’ pAb on CLPTM1

Materials and Methods

Peptide Array

The N-terminal aa 1-354 aa of CLPTM1 was divided with complete coverage into 86 unique 15-mers with 11 aa overlap, produced by JPT peptide Technologies GmbH. The Bioss 8018R polyclonal antibody was incubated at 1 μg/ml o/n at +4 C, washed extensively in PBST followed by 1 hr incubation RT with Alexa flour 647 (Thermo scientific) Rabbit IgG (H+L) Polyclonal Secondary Antibody (Catalog#: A-21244) diluted 1:60000 and after repeated PBST wash detected using a G2502 Microarray scanner (Agilent Technologies). The polypeptides used and their SEQ ID reference numbers are shown in FIG. 32.

The epitope 1 for Bioss BS8018R has the highest Median fluorescence intensity (MFI) (signal intensity) on array, demonstrating highest binding affinity for this site. Binding of the antibody to the various peptides in the array (SEQ ID NOs:274-279) is shown in FIG. 32A.

Minimal common denominator: PKD (SEQ ID NO:322)

Most likely flanking amino acids also participate within the region:

(SEQ ID NO: 313)
GGAPRVASRNLFPKDTLMNLHVYISEH.

The epitope 2 displayed a slightly lower MFI, yet suggest a second site with affinity for this antibody. Binding of the antibody to the various peptides in the array (SEQ ID NOs:280-286) is shown in FIG. 32B. We noticed that this site partly overlap with a binding site for MIC1. Thus the Bioss antibody with this binding pattern could be an antagonist for MIC, by direct competition for MIC1 binding at epitope 2 or by steric hindrance through binding to epitope1. The region for epitope 2 is within:

(SEQ ID NO: 314)
LDQYVKFDAVSGDYYPIIYFNDYWNLQKDYYPINE

The polypeptide from CLPTM1 with the highest affinity for Mid from a panel of 24 different CLPTM1 derived peptides (SEQ ID NO:270) thus appears to overlap this epitope for the Bioss antibody.

### Example 20—Putative Conserved Sequence Between CLPTM1 and QRFPR

A sequence derived from the extracellular domain of QRFPR that was found to have affinity for GDF15 in the array assays performed in Example 7 (see FIG. 12) and in the pull-down assays, was found to have a degree of sequence identity with portions of the extracellular domain of CLPTM1 when a BLAST alignment was performed.

| Score | Expect | Method | Identities | Positives | Gaps | Frame |
|---|---|---|---|---|---|---|
| 16.5 | 1.5( ) | Compositional matrix | 5/15(33%) | 9/15(60%) | 0/15(0%) | |
| bits (31) | | adjust. | | | | |

| | | | | |
|---|---|---|---|---|
| Features: | | | | |
| CLPTM1 | 123 | LFWEQHDLVYGDWTS | 137 | (SEQ ID NO: 125) |
| | | L+ ++H +WTS | | |
| QRFPR | 193 | LYEKEHICCLEEWTS | 207 | (SEQ ID NO: 324) |

| Score | Expect | Method | Identities | Positives | Gaps | Frame |
|---|---|---|---|---|---|---|
| 13.1 | 1.6( ) | Composition-based | 4/25(16%) | 12/25(48%) | 0/25(0%) | |
| bits (22) | | stats. | | | | |

| | | | | |
|---|---|---|---|---|
| Features: | | | | |
| CLPTM1 | 107 | YISEHEHFTDFNATSALFWEQ | 127 | (SEQ ID NO: 96) |
| | | ++ E EH ++ ++ | | |
| QRFPR | 192 | FLYEKEHICCLEEWTSPVHQK | 212 | (SEQ ID NO: 60) |

A polypeptide having or comprising a sequence as shown in SEQ ID NO: 324, or a sequence having at least 75% sequence identity thereto, or a sequence which is a part of SEQ ID NO: 324 comprising at least 6 contiguous amino acids represents a further exemplary polypeptide according to the invention.

1.-62. (canceled)

63. A method of treating or preventing a condition associated with elevated or unwanted levels of GDF15, which method comprises administering to a subject in need thereof an effective amount of a polypeptide capable of binding to GDF15 and inhibiting its interaction with the receptors QRFPR and/or CLPTM1, wherein said polypeptide:
(i) has or comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1), or an amino acid sequence having at least 75% sequence identity to SEQ ID NO:5, 12 or 14; or
(ii) is or comprises part of SEQ ID NO:5, 12 or 14, said part comprising at least 6 contiguous amino acids of SEQ ID NO:5, 12 or 14; or
(iii) comprises at least 6 amino acids corresponding to at least 6 contiguous amino acids of SEQ ID NO:5, 12 or 14 and has at least 75% sequence identity to the equivalent amino acid sequence in SEQ ID NO:5, 12 or 14;
and wherein said polypeptide is provided as a fusion protein comprising said polypeptide and a polypeptide sequence from a different source.

64. The method of claim 63, wherein said polypeptide does not consist of the amino acid sequence set forth in any one of SEQ ID NOs:5, 12, 14 or 315.

65. The method of claim 63, wherein said polypeptide:
(i) comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1) or SEQ ID NO:315 (extracellular domain of CLPTM1 with N-terminal methionine), wherein the amino acid sequence is not flanked at either or both ends by an amino acid sequence which flanks the said amino acid sequence in a native QRFPR or CLPTM1 receptor, or comprises an amino acid sequence which is not the amino acid sequence of SEQ ID NOs: 5, 12, 14, or 315 but which has at least 75% sequence identity to SEQ ID NO:5, 12, 14, or 315 wherein optionally the amino acid sequence is not flanked at either or both ends by an amino acid sequence which flanks the said amino acid sequence in a native QRFPR or CLPTM1 receptor; or
(ii) is or comprises a part of SEQ ID NO:5, 12, 14 or 315, said part comprising at least 6 contiguous amino acids of SEQ ID NO:5, 12, 14 or 315 wherein (i) at least 2 contiguous or non-contiguous amino acids of SEQ ID NO:5, 12, 14 or 315 are deleted and/or (ii) said part is not flanked at either or both ends by an amino acid sequence which flanks the said amino acid sequence in a native QRFPR or CLPTM1 receptor; or
(iii) comprises an amino acid sequence having at least 6 amino acids corresponding to at least 6 contiguous amino acids of SEQ ID NO:5, 12, 14 or 315 and has at least 75% sequence identity to the equivalent amino acid sequence in SEQ ID NO:5, 12, 14 or 315, wherein (i) at least 2 contiguous or non-contiguous amino acids of SEQ ID NO:5, 12, 14 or 315 are deleted and/or (ii) the amino acid sequence corresponding to SEQ ID NO:5, 12, 14 or 315 is not flanked at either or both ends by an amino acid sequence which flanks the said amino acid sequence in a native QRFPR or CLPTM1 receptor.

66. The method of claim 63, wherein said polypeptide comprises the sequence FLYEK (SEQ ID NO. 210) corresponding to residues 8 to 13 of SEQ ID NO:5.

67. The method of claim 66, wherein said polypeptide comprises the sequence LEIKYDFLYEKEH (SEQ ID NO:180) corresponding to residues 3 to 15 of SEQ ID NO:5.

68. The method of claim 66, wherein said polypeptide further comprises the sequence WTS (SEQ ID NO. 211) corresponding to residues 22 to 24 of SEQ ID NO. 5.

69. The method of claim 66, wherein said polypeptide has or comprises the sequence as set forth in SEQ ID NO:11 (FLYEKEHIC) or a sequence having at least 75% sequence identity thereto.

70. The method of claim 66, wherein the polypeptide has or comprises a sequence selected from the group consisting of SEQ ID NO: 6, 7, 8 and 9, or a sequence which has at least 75% sequence identity thereto.

71. The method of claim 63, wherein the polypeptide comprises the sequence X₁X₂X₃X₄X₅X₆X₇X₈X₉; wherein:
X₁ is an aromatic amino acid;
X₂ is an aliphatic amino acid;
X₅ is a basic amino acid; and
X₃, X₄, X₆, X₇, X₈ and X₉ may independently be any amino acid (SEQ ID NO:337).

72. The method of claim 71, wherein:
X₁ is F, W or Y;
X₂ is L, V, I, A or M, preferably L, V or I;
X₅ is K, R or H, preferably K or R (SEQ ID NO:338).

73. The method of claim 71, wherein;
X₃ is an aromatic amino acid, preferably F, W or Y; and/or
X₄ is an acidic amino acid, preferably E or D (SEQ ID NOs:339-344).

74. The method of claim 71, wherein:
X₆ is an acidic amino acid, preferably E or D; and/or
X₇ is a basic amino acid, preferably K, R or H, more preferably K or R (SEQ ID NOs:345-369).

75. The method of claim 71, wherein:
X₈ is an aliphatic amino acid, preferably L, V, I, A or M, more preferably L, V or I; and/or
X₉ is any amino acid, preferably C (SEQ ID NOs:370-402).

76. The method of claim 71, wherein the polypeptide comprises the sequence X₁X₂X₃X₄X₅X₆X₇X₈X₉; wherein:
X₁ is F, W or Y;
X₂ is L, V or I;
X₃ is F, W or Y;
X₄ 1 S D or E;
X₅ is K, R or H;
X₆ 1 S D or E;
X₇ is K, R or H;
X₈ is L, V or I; and
X₉ is C (SEQ ID NO:403).

77. The method of claim 71, wherein the sequence X₁X₂X₃X₄X₅X₆X₇X₈X₉ is flanked on one or both sides by 1 to 10 amino acids, preferably wherein said flanking amino acid sequences comprise amino acids capable of forming an α-helix or a β-sheet.

78. The method of claim 77, wherein residue X₁ is flanked by an amino acid sequence comprising amino acids capable of forming an α-helix and/or residue X₉ is flanked by an amino acid sequence comprising amino acids capable of forming a β-sheet, preferably wherein X₉ is flanked by C.

79. The method of claim 63, wherein the polypeptide has or comprises the sequence as set forth in SEQ ID NO:13 (EKEYDDVTIK) or a sequence having at least 75% sequence identity thereto.

80. The method of claim 63, wherein the polypeptide comprises the sequence X₁X₂X₃X₄X₅X₆X₇X₈X₉X₁₀, wherein X₁ is D or E; X₂ is K, R or H; X₃ is D or E; X₄ is F, W or Y; X₅ is D or E; X₆ is D or E; X₇ is L, V or I; X₈ is A, S or T; X₉ is L, V or I; and X₁₀ is R, K or H (SEQ ID NO:405).

81. The method of claim 63, wherein the polypeptide has or comprises the sequence as set forth in SEQ ID NO:15 (YISEHEH) or a sequence having at least 75% sequence identity thereto, and/or wherein the polypeptide has or comprises the sequence as set forth in SEQ ID NO:16 (LFWEQH), or a sequence having at least 75% sequence identity thereto.

82. The method of claim 63, wherein the polypeptide has or comprises a sequence as set forth in SEQ ID NO:17, or sequence having at least 75% sequence identity thereto, or a sequence which is a part of SEQ ID NO:17 comprising at least 6 contiguous amino acids.

83. The method of claim 63, wherein the polypeptide has or comprises the sequence of SEQ ID NO:18 or a sequence having at least 75% sequence identity thereto.

84. The method of claim 63, wherein the polypeptide has or comprises the sequence as set forth in any one of SEQ ID NOs:258, 259, 262, 263, 269, 270, 297, 298, 301, 302, 308 or 309 or a sequence having at least 75% sequence identity thereto or sequence which is a part of any aforesaid sequence comprising at least 6 contiguous amino acids.

85. The method of claim 63, wherein the polypeptide for use or polypeptide has or comprises the sequence as set forth in any one of SEQ ID NOs:30, 31, 32, 39, 46, 47 or 175-189 or a sequence having at least 75% sequence identity thereto.

86. The method of claim 63, wherein the polypeptide is in the form of a dimer or higher multimeric form.

87. The method of claim 63, wherein the fusion partner in said fusion protein is albumin, transferrin, an immunoglobulin or part thereof, fibrinogen, a homo amino acid polymer, a proline-alanine-serine polymer, or an elastin-like peptide, wherein the fusion partner is optionally linked by a linker sequence.

88. The method of claim 63, wherein the condition is selected from one or more of cancer, cachexia, a bone disorder, cardiovascular disease, a chronic pulmonary disorder, pulmonary arterial hypertension, a renal disorder, sickle cell disease, hereditary spherocytosis or an iron overload disorder.

89. The method of claim 63, wherein the polypeptide is for reducing the immunosuppressive effects of GDF15.

90. The method of claim 88, for the treatment or prevention of cancer metastasis, particularly for treating or preventing the engagement of bone in cancer.

91. The method of claim 88, wherein said cachexia is cancer-induced.

92. The method of claim 63, wherein the polypeptide does not consist of the amino acid sequence set forth in any one of SEQ ID NOs: 243-245.

93. The method of claim 63, wherein the fusion partner in said fusion protein is an Fc region of an immunoglobulin.

94. A polypeptide capable of binding to GDF15 and inhibiting its interaction with the receptors QRFPR and/or CLPTM1, wherein said polypeptide:
(i) has or comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1), or an amino acid sequence having at least 75% sequence identity to SEQ ID NO:5, 12 or 14; or
(ii) is or comprises part of SEQ ID NO:5, 12 or 14, said part comprising at least 6 contiguous amino acids of SEQ ID NO:5, 6 or 9; or
(iii) comprises at least 6 amino acids corresponding to at least 6 contiguous amino acids of SEQ ID NO:5, 12 or 14 and has at least 75% sequence identity to the equivalent amino acid sequence in SEQ ID NO:5, 12 or 14;
wherein said polypeptide is provided as a fusion protein comprising said polypeptide fused with the Fc region of an immunoglobulin, optionally linked by a linker sequence.

95. A pharmaceutical composition comprising a polypeptide as defined in claim 94, together with at least one pharmaceutically-acceptable carrier or excipient.

96. A method of treating cancer, said method comprising administering to a subject in need thereof an effective amount of a polypeptide, wherein said polypeptide:
(i) has or comprises an amino acid sequence as set forth in SEQ ID NO:5 (extracellular domain 3 of QRFPR), SEQ ID NO:12 (extracellular domain 4 of QRFPR) or SEQ ID NO:14 (extracellular domain of CLPTM1), or an amino acid sequence having at least 75% sequence identity to SEQ ID NO:5, 12 or 14; or
(ii) is or comprises part of SEQ ID NO:5, 12 or 14, said part comprising at least 6 contiguous amino acids of SEQ ID NO:5, 12 or 14; or
(iii) comprises at least 6 amino acids corresponding to at least 6 contiguous amino acids of SEQ ID NO:5, 12 or 14 and has at least 75% sequence identity to the equivalent amino acid sequence in SEQ ID NO:5, 12 or 14;
and wherein said polypeptide is provided as a fusion protein comprising said polypeptide fused with an immunoglobulin or part thereof, albumin or transferrin, optionally linked by a linker sequence.

97. The method of claim 96, wherein the polypeptide is fused with an Fc region of an immunoglobulin.


SEQUENCE LISTING (405 sequences)

SEQ ID NO: 1 - protein, 431 aa, Homo sapiens
MQALNITPEQFSRLLRDHNLTREQFIALYRLRPLVYTPELPGRAKLALVLTGVLIFALALFGNALVFYVVTRSKAMRTVTNIFICSLALSDLLITFFCIPVTMLQNISDNWLGGAFICKMVPFVQSTAVVTEILTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAVIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 2 - protein, 669 aa, Homo sapiens
MAAAQEADGARSAVVAAGGGSSGQVTSNGSIGRDPPAETQPQNPPAQPAPNAWQVIKGVLFRIFIIWAISSWFRRGPAPQDQAGPGGAPRVASRNLFPKDTLMNLHVYISEHEHFTDFNATSALFWEQHDLVYGDWTSGENSDGCYEHFAELDIPQSVQQNGSIYIHVYFTKSGFHPDPRQKALYRRLATVHMSRMINKYKRRRFQKTKNLLTGETEADPEMIKRAEDYGPVEVISHWHPNITINIVDDHTPWVKGSVPPPLDQYVKFDAVSGDYYPIIYFNDYWNLQKDYYPINESLASLPLRVSFCPLSLWRWQLYAAQSTKSPWNFLGDELYEQSDEEQDSVKVALLETNPYLLALTIIVSIVHSVFEFLAFKNDIQFWNSRQSLEGLSVRSVFFGVFQSFVVLLYILDNETNFVVQVSVFIGVLIDLWKITKVMDVRLDREHRVAGIFPRLSFKDKSTYIESSTKVYDDMAFRYLSWILFPLLGCYAVYSLLYLEHKGWYSWVLSMLYGFLLTFGFITMTPQLFINYKLKSVAHLPWRMLTYKALNTFIDDLFAFVIKMPVMYRIGCLRDDVVFFIYLYQRWIYRVDPTRVNEFGMSGEDPTAAAPVAEVPTAAGALTPTPAPTTTTATREEASTSLPTKPTQGASSASEPQEAPPKPAEDKKKD

SEQ ID NO: 3 - protein, 46 aa, Homo sapiens
MQALNITPEQFSRLLRDHNLTREQFIALYRLRPLVYTPELPGRAKL

SEQ ID NO: 4 - protein, 18 aa, Homo sapiens
MLQNISDNWLGGAFICKM

SEQ ID NO: 5 - protein, 29 aa, Homo sapiens
QQLEIKYDFLYEKEHICCLEEWTSPVHQK

SEQ ID NO: 6 - protein, 26 aa, Homo sapiens
EIKYDFLYEKEHICCLEEWTSPVHQK

SEQ ID NO: 7 - protein, 21 aa, Homo sapiens
EIKYDFLYEKEHICCLEEWTS

SEQ ID NO: 8 - protein, 16 aa, Homo sapiens
FLYEKEHICCLEEWTS

SEQ ID NO: 9 - protein, 11 aa, Homo sapiens
FLYEKEHICCL

SEQ ID NO: 10 - protein, 22 aa, Homo sapiens
GEIKYDFLYEKEHICCLEEWTS

SEQ ID NO: 11 - protein, 9 aa, Homo sapiens
FLYEKEHIC

SEQ ID NO: 12 - protein, 19 aa, Homo sapiens
HMMIEYSNFEKEYDDVTIK

SEQ ID NO: 13 - protein, 10 aa, Homo sapiens
EKEYDDVTIK

SEQ ID NO: 14 - protein, 353 aa, Homo sapiens
AAAQEADGARSAVVAAGGGSSGQVTSNGSIGRDPPAETQPQNPPAQPAPNAWQVIKGVLFRIFIIWAISSWFRRGPAPQDQAGPGGAPRVASRNLFPKDTLMNLHVYISEHEHFTDFNATSALFWEQHDLVYGDWTSGENSDGCYEHFAELDIPQSVQQNGSIYIHVYFTKSGFHPDPRQKALYRRLATVHMSRMINKYKRRRFQKTKNLLTGETEADPEMIKRAEDYGPVEVISHWHPNITINIVDDHTPWVKGSVPPPLDQYVKFDAVSGDYYPIIYFNDYWNLQKDYYPINESLASLPLRVSFCPLSLWRWQLYAAQSTKSPWNFLGDELYEQSDEEQDSVKVALLETNP

SEQ ID NO: 15 - protein, 7 aa, Homo sapiens
YISEHEH

SEQ ID NO: 16 - protein, 6 aa, Homo sapiens
LFWEQH

SEQ ID NO: 17 - protein, 31 aa, Homo sapiens
YISEHEHFTDFNATSALFWEQHDLVYGDWTS

SEQ ID NO: 18 - protein, 16 aa, Homo sapiens
ALFWEQHDLVYGDWTS

SEQ ID NO: 19 - protein, 17 aa, Homo sapiens
GALFWEQHDLVYGDWTS

SEQ ID NO: 20 - protein, 28 aa, Homo sapiens
QQLEIKYDFLYEKEHICCLEEWTSPVHQ

SEQ ID NO: 21 - protein, 27 aa, Homo sapiens
QQLEIKYDFLYEKEHICCLEEWTSPVH

SEQ ID NO: 22 - protein, 26 aa, Homo sapiens
QQLEIKYDFLYEKEHICCLEEWTSPV

SEQ ID NO: 23 - protein, 25 aa, Homo sapiens
QQLEIKYDFLYEKEHICCLEEWTSP

SEQ ID NO: 24 - protein, 24 aa, Homo sapiens
QQLEIKYDFLYEKEHICCLEEWTS

SEQ ID NO: 25 - protein, 23 aa, Homo sapiens
QQLEIKYDFLYEKEHICCLEEWT

SEQ ID NO: 26 - protein, 22 aa, Homo sapiens
QQLEIKYDFLYEKEHICCLEEW

SEQ ID NO: 27 - protein, 21 aa, Homo sapiens
QQLEIKYDFLYEKEHICCLEE

SEQ ID NO: 28 - protein, 20 aa, Homo sapiens
QQLEIKYDFLYEKEHICCLE

SEQ ID NO: 29 - protein, 19 aa, Homo sapiens
QQLEIKYDFLYEKEHICCL

SEQ ID NO: 30 - protein, 18 aa, Homo sapiens
QQLEIKYDFLYEKEHICC

SEQ ID NO: 31 - protein, 17 aa, Homo sapiens
QQLEIKYDFLYEKEHIC

SEQ ID NO: 32 - protein, 17 aa, Homo sapiens
QLEIKYDFLYEKEHICC

SEQ ID NO: 33 - protein, 17 aa, Homo sapiens
LEIKYDFLYEKEHICCL

SEQ ID NO: 34 - protein, 17 aa, Homo sapiens
EIKYDFLYEKEHICCLE

SEQ ID NO: 35 - protein, 17 aa, Homo sapiens
IKYDFLYEKEHICCLEE

SEQ ID NO: 36 - protein, 17 aa, Homo sapiens
KYDFLYEKEHICCLEEW

SEQ ID NO: 37 - protein, 17 aa, Homo sapiens
YDFLYEKEHICCLEEWT

SEQ ID NO: 38 - protein, 17 aa, Homo sapiens
DFLYEKEHICCLEEWTS

SEQ ID NO: 39 - protein, 15 aa, Homo sapiens
QLEIKYDFLYEKEHI

SEQ ID NO: 40 - protein, 15 aa, Homo sapiens
LEIKYDFLYEKEHIC

SEQ ID NO: 41 - protein, 15 aa, Homo sapiens
EIKYDFLYEKEHICC

SEQ ID NO: 42 - protein, 15 aa, Homo sapiens
IKYDFLYEKEHICCL

SEQ ID NO: 43 - protein, 15 aa, Homo sapiens
KYDFLYEKEHICCLE

SEQ ID NO: 44 - protein, 15 aa, Homo sapiens
YDFLYEKEHICCLEE

SEQ ID NO: 45 - protein, 15 aa, Homo sapiens
DFLYEKEHICCLEEW

SEQ ID NO: 46 - protein, 16 aa, Homo sapiens
QLEIKYDFLYEKEHIC

SEQ ID NO: 47 - protein, 15 aa, Homo sapiens
LEIKYDFLYEKEHIC

SEQ ID NO: 48 - protein, 14 aa, Homo sapiens
EIKYDFLYEKEHIC

SEQ ID NO: 49 - protein, 13 aa, Homo sapiens
IKYDFLYEKEHIC

SEQ ID NO: 50 - protein, 12 aa, Homo sapiens
KYDFLYEKEHIC

SEQ ID NO: 51 - protein, 11 aa, Homo sapiens
YDFLYEKEHIC

SEQ ID NO: 52 - protein, 10 aa, Homo sapiens
DFLYEKEHIC

SEQ ID NO: 53 - protein, 28 aa, Homo sapiens
QLEIKYDFLYEKEHICCLEEWTSPVHQK

SEQ ID NO: 54 - protein, 27 aa, Homo sapiens
LEIKYDFLYEKEHICCLEEWTSPVHQK

SEQ ID NO: 55 - protein, 26 aa, Homo sapiens
EIKYDFLYEKEHICCLEEWTSPVHQK

SEQ ID NO: 56 - protein, 25 aa, Homo sapiens
IKYDFLYEKEHICCLEEWTSPVHQK

SEQ ID NO: 57 - protein, 24 aa, Homo sapiens
KYDFLYEKEHICCLEEWTSPVHQK

SEQ ID NO: 58 - protein, 23 aa, Homo sapiens
YDFLYEKEHICCLEEWTSPVHQK

SEQ ID NO: 59 - protein, 22 aa, Homo sapiens
DFLYEKEHICCLEEWTSPVHQK

SEQ ID NO: 60 - protein, 21 aa, Homo sapiens
FLYEKEHICCLEEWTSPVHQK

SEQ ID NO: 61 - protein, 21 aa, Homo sapiens
DFLYEKEHICCLEEWTSPVHQ

SEQ ID NO: 62 - protein, 21 aa, Homo sapiens
YDFLYEKEHICCLEEWTSPVH

SEQ ID NO: 63 - protein, 21 aa, Homo sapiens
KYDFLYEKEHICCLEEWTSPV

SEQ ID NO: 64 - protein, 21 aa, Homo sapiens
IKYDFLYEKEHICCLEEWTSP

SEQ ID NO: 65 - protein, 21 aa, Homo sapiens
EIKYDFLYEKEHICCLEEWTS

SEQ ID NO: 66 - protein, 21 aa, Homo sapiens
LEIKYDFLYEKEHICCLEEWT

SEQ ID NO: 67 - protein, 21 aa, Homo sapiens
QLEIKYDFLYEKEHICCLEEW

SEQ ID NO: 68 - protein, 20 aa, Homo sapiens
FLYEKEHICCLEEWTSPVHQ

SEQ ID NO: 69 - protein, 19 aa, Homo sapiens
FLYEKEHICCLEEWTSPVH

SEQ ID NO: 70 - protein, 18 aa, Homo sapiens
FLYEKEHICCLEEWTSPV

SEQ ID NO: 71 - protein, 17 aa, Homo sapiens
FLYEKEHICCLEEWTSP

SEQ ID NO: 72 - protein, 16 aa, Homo sapiens
FLYEKEHICCLEEWTS

SEQ ID NO: 73 - protein, 15 aa, Homo sapiens
FLYEKEHICCLEEWT

SEQ ID NO: 74 - protein, 14 aa, Homo sapiens
FLYEKEHICCLEEW

SEQ ID NO: 75 - protein, 13 aa, Homo sapiens
FLYEKEHICCLEE

SEQ ID NO: 76 - protein, 12 aa, Homo sapiens
FLYEKEHICCLE

SEQ ID NO: 77 - protein, 11 aa, Homo sapiens
FLYEKEHICCL

SEQ ID NO: 78 - protein, 10 aa, Homo sapiens
FLYEKEHICC

SEQ ID NO: 79 - protein, 18 aa, Homo sapiens
MMIEYSNFEKEYDDVTIK

SEQ ID NO: 80 - protein, 17 aa, Homo sapiens
MIEYSNFEKEYDDVTIK

SEQ ID NO: 81 - protein, 16 aa, Homo sapiens
IEYSNFEKEYDDVTIK

SEQ ID NO: 82 - protein, 15 aa, Homo sapiens
EYSNFEKEYDDVTIK

SEQ ID NO: 83 - protein, 14 aa, Homo sapiens
YSNFEKEYDDVTIK

SEQ ID NO: 84 - protein, 13 aa, Homo sapiens
SNFEKEYDDVTIK

SEQ ID NO: 85 - protein, 12 aa, Homo sapiens
NFEKEYDDVTIK

SEQ ID NO: 86 - protein, 11 aa, Homo sapiens
FEKEYDDVTIK

SEQ ID NO: 87 - protein, 30 aa, Homo sapiens
YISEHEHFTDFNATSALFWEQHDLVYGDWT

SEQ ID NO: 88 - protein, 29 aa, Homo sapiens
YISEHEHFTDFNATSALFWEQHDLVYGDW

SEQ ID NO: 89 - protein, 28 aa, Homo sapiens
YISEHEHFTDFNATSALFWEQHDLVYGD

SEQ ID NO: 90 - protein, 27 aa, Homo sapiens
YISEHEHFTDFNATSALFWEQHDLVYG

SEQ ID NO: 91 - protein, 26 aa, Homo sapiens
YISEHEHFTDFNATSALFWEQHDLVY

SEQ ID NO: 92 - protein, 25 aa, Homo sapiens
YISEHEHFTDFNATSALFWEQHDLV

SEQ ID NO: 93 - protein, 24 aa, Homo sapiens
YISEHEHFTDFNATSALFWEQHDL

SEQ ID NO: 94 - protein, 23 aa, Homo sapiens
YISEHEHFTDFNATSALFWEQHD

SEQ ID NO: 95 - protein, 22 aa, Homo sapiens
YISEHEHFTDFNATSALFWEQH

SEQ ID NO: 96 - protein, 21 aa, Homo sapiens
YISEHEHFTDFNATSALFWEQ

SEQ ID NO: 97 - protein, 20 aa, Homo sapiens
YISEHEHFTDFNATSALFWE

SEQ ID NO: 98 - protein, 19 aa, Homo sapiens
YISEHEHFTDFNATSALFW

SEQ ID NO: 99 - protein, 18 aa, Homo sapiens
YISEHEHFTDFNATSALF

SEQ ID NO: 100 - protein, 17 aa, Homo sapiens
YISEHEHFTDFNATSAL

SEQ ID NO: 101 - protein, 16 aa, Homo sapiens
YISEHEHFTDFNATSA

SEQ ID NO: 102 - protein, 15 aa, Homo sapiens
YISEHEHFTDFNATS

SEQ ID NO: 103 - protein, 14 aa, Homo sapiens
YISEHEHFTDFNAT

SEQ ID NO: 104 - protein, 13 aa, Homo sapiens
YISEHEHFTDFNA

SEQ ID NO: 105 - protein, 12 aa, Homo sapiens
YISEHEHFTDFN

SEQ ID NO: 106 - protein, 11 aa, Homo sapiens
YISEHEHFTDF

SEQ ID NO: 107 - protein, 10 aa, Homo sapiens
YISEHEHFTD

SEQ ID NO: 108 - protein, 9 aa, Homo sapiens
YISEHEHFT

SEQ ID NO: 109 - protein, 8 aa, Homo sapiens
YISEHEHF

SEQ ID NO: 110 - protein, 30 aa, Homo sapiens
ISEHEHFTDFNATSALFWEQHDLVYGDWTS

SEQ ID NO: 111 - protein, 29 aa, Homo sapiens
SEHEHFTDFNATSALFWEQHDLVYGDWTS

SEQ ID NO: 112 - protein, 28 aa, Homo sapiens
EHEHFTDFNATSALFWEQHDLVYGDWTS

SEQ ID NO: 113 - protein, 27 aa, Homo sapiens
HEHFTDFNATSALFWEQHDLVYGDWTS

SEQ ID NO: 114 - protein, 26 aa, Homo sapiens
EHFTDFNATSALFWEQHDLVYGDWTS

SEQ ID NO: 115 - protein, 25 aa, Homo sapiens
HFTDFNATSALFWEQHDLVYGDWTS

SEQ ID NO: 116 - protein, 24 aa, Homo sapiens
FTDFNATSALFWEQHDLVYGDWTS

SEQ ID NO: 117 - protein, 23 aa, Homo sapiens
TDFNATSALFWEQHDLVYGDWTS

SEQ ID NO: 118 - protein, 22 aa, Homo sapiens
DFNATSALFWEQHDLVYGDWTS

SEQ ID NO: 119 - protein, 21 aa, Homo sapiens
FNATSALFWEQHDLVYGDWTS

SEQ ID NO: 120 - protein, 20 aa, Homo sapiens
NATSALFWEQHDLVYGDWTS

SEQ ID NO: 121 - protein, 19 aa, Homo sapiens
ATSALFWEQHDLVYGDWTS

SEQ ID NO: 122 - protein, 18 aa, Homo sapiens
TSALFWEQHDLVYGDWTS

SEQ ID NO: 123 - protein, 17 aa, Homo sapiens
SALFWEQHDLVYGDWTS

SEQ ID NO: 124 - protein, 16 aa, Homo sapiens
ALFWEQHDLVYGDWTS

SEQ ID NO: 125 - protein, 15 aa, Homo sapiens
LFWEQHDLVYGDWTS

SEQ ID NO: 126 - protein, 14 aa, Homo sapiens
LFWEQHDLVYGDWT

SEQ ID NO: 127 - protein, 13 aa, Homo sapiens
LFWEQHDLVYGDW

SEQ ID NO: 128 - protein, 12 aa, Homo sapiens
LFWEQHDLVYGD

SEQ ID NO: 129 - protein, 11 aa, Homo sapiens
LFWEQHDLVYG

SEQ ID NO: 130 - protein, 10 aa, Homo sapiens
LFWEQHDLVY

SEQ ID NO: 131 - protein, 9 aa, Homo sapiens
LFWEQHDLV

SEQ ID NO: 132 - protein, 8 aa, Homo sapiens
LFWEQHDL

SEQ ID NO: 133 - protein, 7 aa, Homo sapiens
LFWEQHD

SEQ ID NO: 134 - protein, 15 aa, Homo sapiens
FTDFNATSALFWEQH

SEQ ID NO: 135 - protein, 15 aa, Homo sapiens
TDFNATSALFWEQHD

SEQ ID NO: 136 - protein, 15 aa, Homo sapiens
DFNATSALFWEQHDL

SEQ ID NO: 137 - protein, 15 aa, Homo sapiens
FNATSALFWEQHDLV

SEQ ID NO: 138 - protein, 15 aa, Homo sapiens
NATSALFWEQHDLVY

SEQ ID NO: 139 - protein, 15 aa, Homo sapiens
ATSALFWEQHDLVYG

SEQ ID NO: 140 - protein, 15 aa, Homo sapiens
TSALFWEQHDLVYGD

SEQ ID NO: 141 - protein, 15 aa, Homo sapiens
SALFWEQHDLVYGDW

SEQ ID NO: 142 - protein, 15 aa, Homo sapiens
ALFWEQHDLVYGDWT

SEQ ID NO: 143 - protein, 21 aa, Homo sapiens
ISEHEHFTDFNATSALFWEQH

SEQ ID NO: 144 - protein, 20 aa, Homo sapiens
SEHEHFTDFNATSALFWEQH

SEQ ID NO: 145 - protein, 19 aa, Homo sapiens
EHEHFTDFNATSALFWEQH

SEQ ID NO: 146 - protein, 18 aa, Homo sapiens
HEHFTDFNATSALFWEQH

SEQ ID NO: 147 - protein, 17 aa, Homo sapiens
EHFTDFNATSALFWEQH

SEQ ID NO: 148 - protein, 16 aa, Homo sapiens
HFTDFNATSALFWEQH

SEQ ID NO: 149 - protein, 15 aa, Homo sapiens
FTDFNATSALFWEQH

SEQ ID NO: 150 - protein, 14 aa, Homo sapiens
TDFNATSALFWEQH

SEQ ID NO: 151 - protein, 13 aa, Homo sapiens
DFNATSALFWEQH

SEQ ID NO: 152 - protein, 12 aa, Homo sapiens
FNATSALFWEQH

SEQ ID NO: 153 - protein, 11 aa, Homo sapiens
NATSALFWEQH

SEQ ID NO: 154 - protein, 10 aa, Homo sapiens
ATSALFWEQH

SEQ ID NO: 155 - protein, 8 aa, Homo sapiens
SALFWEQH

SEQ ID NO: 156 - protein, 7 aa, Homo sapiens
ALFWEQH

SEQ ID NO: 157 - protein, 22 aa, Homo sapiens
ISEHEHFTDFNATSALFWEQHD

SEQ ID NO: 158 - protein, 22 aa, Homo sapiens
SEHEHFTDFNATSALFWEQHDL

SEQ ID NO: 159 - protein, 22 aa, Homo sapiens
EHEHFTDFNATSALFWEQHDLV

SEQ ID NO: 160 - protein, 22 aa, Homo sapiens
HEHFTDFNATSALFWEQHDLVY

SEQ ID NO: 161 - protein, 22 aa, Homo sapiens
EHFTDFNATSALFWEQHDLVYG

SEQ ID NO: 162 - protein, 22 aa, Homo sapiens
HFTDFNATSALFWEQHDLVYGD

SEQ ID NO: 163 - protein, 22 aa, Homo sapiens
FTDFNATSALFWEQHDLVYGDW

SEQ ID NO: 164 - protein, 22 aa, Homo sapiens
TDFNATSALFWEQHDLVYGDWT

SEQ ID NO: 165 - protein, 22 aa, Homo sapiens
DFNATSALFWEQHDLVYGDWTS

SEQ ID NO: 166 - protein, 15 aa, Homo sapiens
SALFWEQHDLVYGDW

SEQ ID NO: 167 - protein, 15 aa, Homo sapiens
TSALFWEQHDLVYGD

SEQ ID NO: 168 - protein, 15 aa, Homo sapiens
ATSALFWEQHDLVYG

SEQ ID NO: 169 - protein, 15 aa, Homo sapiens
NATSALFWEQHDLVY

SEQ ID NO: 170 - protein, 15 aa, Homo sapiens
FNATSALFWEQHDLV

SEQ ID NO: 171 - protein, 15 aa, Homo sapiens
DFNATSALFWEQHDL

SEQ ID NO: 172 - protein, 15 aa, Homo sapiens
TDFNATSALFWEQHD

SEQ ID NO: 173 - protein, 15 aa, Homo sapiens
FTDFNATSALFWEQH

SEQ ID NO: 174 - protein, 17 aa, Homo sapiens
GIEYSNFEKEYDDVTIK

SEQ ID NO: 175 - protein, 16 aa, Homo sapiens
QQLEIKYDFLYEKEHI

SEQ ID NO: 176 - protein, 15 aa, Homo sapiens
QQLEIKYDFLYEKEH

SEQ ID NO: 177 - protein, 14 aa, Homo sapiens
QLEIKYDFLYEKEH

SEQ ID NO: 178 - protein, 16 aa, Homo sapiens
LEIKYDFLYEKEHICC

SEQ ID NO: 179 - protein, 14 aa, Homo sapiens
LEIKYDFLYEKEHI

SEQ ID NO: 180 - protein, 13 aa, Homo sapiens
LEIKYDFLYEKEH

SEQ ID NO: 181 - protein, 18 aa, Mus musculus
QRLEIKYDFLYEKEHICC

SEQ ID NO: 182 - protein, 17 aa, Mus musculus
QRLEIKYDFLYEKEHIC

SEQ ID NO: 183 - protein, 16 aa, Mus musculus
QRLEIKYDFLYEKEHI

SEQ ID NO: 184 - protein, 15 aa, Mus musculus
QRLEIKYDFLYEKEH

SEQ ID NO: 185 - protein, 17 aa, Mus musculus
RLEIKYDFLYEKEHICC

SEQ ID NO: 186 - protein, 16 aa, Mus musculus
LEIKYDFLYEKEHICC

SEQ ID NO: 187 - protein, 16 aa, Mus musculus
RLEIKYDFLYEKEHIC

SEQ ID NO: 188 - protein, 15 aa, Mus musculus
RLEIKYDFLYEKEHI

SEQ ID NO: 189 - protein, 14 aa, Mus musculus
RLEIKYDFLYEKEH

SEQ ID NO: 190 - protein, 16 aa, Homo sapiens
SALFWEQHDLVYGDWT

SEQ ID NO: 191 - protein, 24 aa, Homo sapiens
FNATSALFWEQHDLVYGDWTSGEN

SEQ ID NO: 192 - protein, 23 aa, Homo sapiens
FNATSALFWEQHDLVYGDWTSGE

SEQ ID NO: 193 - protein, 22 aa, Homo sapiens
FNATSALFWEQHDLVYGDWTSG

SEQ ID NO: 194 - protein, 21 aa, Homo sapiens
FNATSALFWEQHDLVYGDWTS

SEQ ID NO: 195 - protein, 20 aa, Homo sapiens
FNATSALFWEQHDLVYGDWT

SEQ ID NO: 196 - protein, 23 aa, Homo sapiens
NATSALFWEQHDLVYGDWTSGEN

SEQ ID NO: 197 - protein, 22 aa, Homo sapiens
ATSALFWEQHDLVYGDWTSGEN

SEQ ID NO: 198 - protein, 21 aa, Homo sapiens
TSALFWEQHDLVYGDWTSGEN

SEQ ID NO: 199 - protein, 20 aa, Homo sapiens
SALFWEQHDLVYGDWTSGEN

SEQ ID NO: 200 - protein, 9 aa, Homo sapiens
YFNDYWNLQ

SEQ ID NO: 201 - protein, 17 aa, Homo sapiens
YPIIYFNDYWNLQKDYY

SEQ ID NO: 202 - protein, 16 aa, Homo sapiens
YPIIYFNDYWNLQKDY

SEQ ID NO: 203 - protein, 15 aa, Homo sapiens
YPIIYFNDYWNLQKD

SEQ ID NO: 204 - protein, 14 aa, Homo sapiens
YPIIYFNDYWNLQK

SEQ ID NO: 205 - protein, 13 aa, Homo sapiens
YPIIYFNDYWNLQ

SEQ ID NO: 206 - protein, 16 aa, Homo sapiens
PIIYFNDYWNLQKDYY

SEQ ID NO: 207 - protein, 15 aa, Homo sapiens
IIYFNDYWNLQKDYY

SEQ ID NO: 208 - protein, 14 aa, Homo sapiens
IYFNDYWNLQKDYY

SEQ ID NO: 209 - protein, 13 aa, Homo sapiens
YFNDYWNLQKDYY

SEQ ID NO: 210 - protein, 5 aa, Homo sapiens
FLYEK

SEQ ID NO: 211 - protein, 3 aa, Homo sapiens
WTS

SEQ ID NO: 212 - protein, 4 aa, Homo sapiens
PKDT

SEQ ID NO: 213 - protein, 14 aa, Homo sapiens
APRVASRNLFPKDT

SEQ ID NO: 214 - protein, 13 aa, Homo sapiens
PRVASRNLFPKDT

SEQ ID NO: 215 - protein, 12 aa, Homo sapiens
RVASRNLFPKDT

SEQ ID NO: 216 - protein, 11 aa, Homo sapiens
VASRNLFPKDT

SEQ ID NO: 217 - protein, 10 aa, Homo sapiens
ASRNLFPKDT

SEQ ID NO: 218 - protein, 9 aa, Homo sapiens
SRNLFPKDT

SEQ ID NO: 219 - protein, 8 aa, Homo sapiens
RNLFPKDT

SEQ ID NO: 220 - protein, 7 aa, Homo sapiens
NLFPKDT

SEQ ID NO: 221 - protein, 6 aa, Homo sapiens
LFPKDT

SEQ ID NO: 222 - protein, 5 aa, Homo sapiens
FPKDT

SEQ ID NO: 223 - protein, 14 aa, Homo sapiens
PRVASRNLFPKDTL

SEQ ID NO: 224 - protein, 13 aa, Homo sapiens
VASRNLFPKDTLM

SEQ ID NO: 225 - protein, 13 aa, Homo sapiens
ASRNLFPKDTLMN

SEQ ID NO: 226 - protein, 13 aa, Homo sapiens
SRNLFPKDTLMNL

SEQ ID NO: 227 - protein, 13 aa, Homo sapiens
RNLFPKDTLMNLH

SEQ ID NO: 228 - protein, 13 aa, Homo sapiens
NLFPKDTLMNLHV

SEQ ID NO: 229 - protein, 13 aa, Homo sapiens
LFPKDTLMNLHVY

SEQ ID NO: 230 - protein, 13 aa, Homo sapiens
FPKDTLMNLHVYI

SEQ ID NO: 231 - protein, 13 aa, Homo sapiens
PKDTLMNLHVYIS

SEQ ID NO: 232 - protein, 12 aa, Homo sapiens
PKDTLMNLHVYI

SEQ ID NO: 233 - protein, 11 aa, Homo sapiens
PKDTLMNLHVY

SEQ ID NO: 234 - protein, 10 aa, Homo sapiens
PKDTLMNLHV

SEQ ID NO: 235 - protein, 9 aa, Homo sapiens
PKDTLMNLH

SEQ ID NO: 236 - protein, 8 aa, Homo sapiens
PKDTLMNL

SEQ ID NO: 237 - protein, 7 aa, Homo sapiens
PKDTLMN

SEQ ID NO: 238 - protein, 6 aa, Homo sapiens
PKDTLM

SEQ ID NO: 239 - protein, 5 aa, Homo sapiens
PKDTL

SEQ ID NO: 240 - protein, 30 aa, Artificial Sequence
  AP5
GGGGSGGGGSGGGGSQRLEIKYDFLYEKEH

SEQ ID NO: 241 - protein, 46 aa, Artificial Sequence
  AP2
GGGGSGGGGSGGGGSYISEHEHFTDFNATSALFWEQHDLVYGDWTS

SEQ ID NO: 242 - protein, 5 aa, Artificial Sequence
  GS linker
GGGGS

SEQ ID NO: 243 - protein, 69 aa, Homo sapiens
RRAFTMLGVVWLVAVIVGSPMWHVQQLEIKYDFLYEKEHICCLEEWTSPVHQKIYTTFILVILFLLPLM

SEQ ID NO: 244 - protein, 134 aa, Homo sapiens
  UNSURE (3)..(3)
  UNSURE (53)..(53)
  UNSURE (68)..(68)
  UNSURE (82)..(82)
FTXSLELSSCNAISPLLYAGHQATHVNLDSLSEDSSQRLFLFRYQMPLPKRSXELFTPSDTCTSLSLXQVTNRSFILYSLKXTYILKYYFISGNFEKEYDDVTIKMIFAIVQIIGFSNSICNPIVYAFMNENFK

SEQ ID NO: 245 - protein, 74 aa, Homo sapiens
KKKRAVIMMVTVVALFAVCWAPFHVVHMMIEYSNFEKEYDDVTIKMIFAIVQIIGFSNSICNPIVYAFMNENFK

SEQ ID NO: 246 - protein, 48 aa, Homo sapiens
QLEIKYDFLYEKEHICCLEEWTSPVHQKIYTTFILSSSSSCLLWKKKR

SEQ ID NO: 247 - protein, 19 aa, Homo sapiens
MMIEYSNFEKEYDDVTIKM

SEQ ID NO: 248 - protein, 28 aa, Homo sapiens
QLEIKYDFLYEKEHICCLEEWTSPVHQK

SEQ ID NO: 249 - protein, 19 aa, Homo sapiens
HMMIEYSNFEKEYDDVTIK

SEQ ID NO: 250 - protein, 21 aa, Homo sapiens
AAAQEADGARSAVVAAGGGSG

SEQ ID NO: 251 - protein, 21 aa, Homo sapiens
SGQVTSNGSIGRDPPAETQPG

SEQ ID NO: 252 - protein, 21 aa, Homo sapiens
QNPPAQPAPNAWQVIKGVLFG

SEQ ID NO: 253 - protein, 21 aa, Homo sapiens
RIFIIWAISSWFRRGPAPQDG

SEQ ID NO: 254 - protein, 21 aa, Homo sapiens
QAGPGGAPRVASRNLFPKDTG

SEQ ID NO: 255 - protein, 21 aa, Homo sapiens
LMNLHVYISEHEHFTDFNATG

SEQ ID NO: 256 - protein, 21 aa, Homo sapiens
SALFWEQHDLVYGDWTSGENG

SEQ ID NO: 257 - protein, 21 aa, Homo sapiens
SDGCYEHFAELDIPQSVQQNG

SEQ ID NO: 258 - protein, 21 aa, Homo sapiens
GSIYIHVYFTKSGFHPDPRQG

SEQ ID NO: 259 - protein, 21 aa, Homo sapiens
KALYRRLATVHMSRMINKYKG

SEQ ID NO: 260 - protein, 21 aa, Homo sapiens
RRRFQKTKNLLTGETEADPEG

SEQ ID NO: 261 - protein, 21 aa, Homo sapiens
MIKRAEDYGPVEVISHWHPNG

SEQ ID NO: 262 - protein, 21 aa, Homo sapiens
ITINIVDDHTPWVKGSVPPPG

SEQ ID NO: 263 - protein, 21 aa, Homo sapiens
LDQYVKFDAVSGDYYPIIYFG

SEQ ID NO: 264 - protein, 21 aa, Homo sapiens
NDYWNLQKDYYPINESLASLG

SEQ ID NO: 265 - protein, 21 aa, Homo sapiens
PLRVSFCPLSLWRWQLYAAQG

SEQ ID NO: 266 - protein, 21 aa, Homo sapiens
STKSPWNFLGDELYEQSDEEG

SEQ ID NO: 267 - protein, 21 aa, Homo sapiens
YEQSDEEQDSVKVALLETNPG

SEQ ID NO: 268 - protein, 21 aa, Homo sapiens
LWRWQLYAAQSTKSPWNFLGG

SEQ ID NO: 269 - protein, 21 aa, Homo sapiens
YPINESLASLPLRVSFCPLSG

SEQ ID NO: 270 - protein, 21 aa, Homo sapiens
GDYYPIIYFNDYWNLQKDYYG

SEQ ID NO: 271 - protein, 21 aa, Homo sapiens
WQVIKGVLFRIFIIWAISSWG

SEQ ID NO: 272 - protein, 21 aa, Homo sapiens
RNLFPKDTLMNLHVYISEHEG

SEQ ID NO: 273 - protein, 21 aa, Homo sapiens
FTDFNATSALFWEQHDLVYGG

SEQ ID NO: 274 - protein, 15 aa, Homo sapiens
QAGPGGAPRVASRNL

SEQ ID NO: 275 - protein, 15 aa, Homo sapiens
GGAPRVASRNLFPKD

SEQ ID NO: 276 - protein, 15 aa, Homo sapiens
RVASRNLFPKDTLMN

SEQ ID NO: 277 - protein, 15 aa, Homo sapiens
RNLFPKDTLMNLHVY

SEQ ID NO: 278 - protein, 15 aa, Homo sapiens
PKDTLMNLHVYISEH

SEQ ID NO: 279 - protein, 15 aa, Homo sapiens
LMNLHVYISEHEHFT

SEQ ID NO: 280 - protein, 15 aa, Homo sapiens
LDQYVKFDAVSGDYY

SEQ ID NO: 281 - protein, 15 aa, Homo sapiens
VKFDAVSGDYYPIIY

SEQ ID NO: 282 - protein, 15 aa, Homo sapiens
AVSGDYYPIIYFNDY

SEQ ID NO: 283 - protein, 15 aa, Homo sapiens
DYYPIIYFNDYWNLQ

SEQ ID NO: 284 - protein, 15 aa, Homo sapiens
IIYFNDYWNLQKDYY

SEQ ID NO: 285 - protein, 15 aa, Homo sapiens
NDYWNLQKDYYPINE

SEQ ID NO: 286 - protein, 15 aa, Homo sapiens
NLQKDYYPINESLAS

SEQ ID NO: 287 - protein, 438 aa, Homo sapiens
MICCSALSPRIHLSFHRSLTGIVLANSSLDIVLHDTYYWAHCGGNVRRHCGGPASRERTAMQALNITPEQFSRLLRDHNLTREQFIALYRLRPLVYTPELPGRAKLALVLTGVLIFALALFGNALVFYWTRSKAMRTVTNIFICSLASDLLITFFCIPVTMLQNISDNLGGAFICEMVPFVQSTAVVTEILTMTCIAVERHQGLVHPFKKWQYTNRRAFTMLGVVLVAVIVGSPMHVQQEIKYDFLYEKEHICCEETSPVHQKIYTTFILSSSSSCLLWKKKRAVIMMVTWALFAVCWAPFHVVHMMIEYSNFEKEYDDVTIKIFAIVQIIGFSNSICNPIVYAFMNENFKKNVLSAVCYCIVNKTFSPAQRHGNSGITMRKKAKFSLRENPVEETKGEAFSDGNIEVKCEQTEEKKKKRHLALFRSELAENSPDSGH

SEQ ID NO: 288 - protein, 419 aa, Homo sapiens
MQALNITPEQFSRLLRDHNLTREQFIAVHRLRPLVYTPELPGRAKLALVLTGVLIFALALFGNALVFYWTRSKAMRTVTNIFICSLASDLLITFFCIPVTMQNISDNLGGAFICKMVPFVQSTAVVTEILTMTCIAVERHQGVHPFKMKQYTNRRAFTLGVVLVAVIVGSPMWHVQQLEIKYDFLYEKEHICCLEETSPVHQKIYTTFILVILFLPLMVMLILYSKIGYELWIKKRVGDGSVLRTIHGKEMSKIARKKIRAVIMMVTVVALFAVCAPFHVVHMMIEYSNFEKEYDDVTIKMIFAIVQIIGFSNSICNPIVYAFNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 289 - protein, 20 aa, Homo sapiens
AAAQEADGARSAVVAAGGGS

SEQ ID NO: 290 - protein, 20 aa, Homo sapiens
SGQVTSNGSIGRDPPAETQP

SEQ ID NO: 291 - protein, 20 aa, Homo sapiens
QNPPAQPAPNAWQVIKGVLF

SEQ ID NO: 292 - protein, 20 aa, Homo sapiens
RIFIIWAISSWFRRGPAPQD

SEQ ID NO: 293 - protein, 20 aa, Homo sapiens
QAGPGGAPRVASRNLFPKDT

SEQ ID NO: 294 - protein, 20 aa, Homo sapiens
LMNLHVYISEHEHFTDFNAT

SEQ ID NO: 295 - protein, 20 aa, Homo sapiens
SALFWEQHDLVYGDWTSGEN

SEQ ID NO: 296 - protein, 20 aa, Homo sapiens
SDGCYEHFAELDIPQSVQQN

SEQ ID NO: 297 - protein, 20 aa, Homo sapiens
GSIYIHVYFTKSGFHPDPRQ

SEQ ID NO: 298 - protein, 20 aa, Homo sapiens
KALYRRLATVHMSRMINKYK

SEQ ID NO: 299 - protein, 20 aa, Homo sapiens
RRRFQKTKNLLTGETEADPE

SEQ ID NO: 300 - protein, 20 aa, Homo sapiens
MIKRAEDYGPVEVISHWHPN

SEQ ID NO: 301 - protein, 20 aa, Homo sapiens
ITINIVDDHTPWVKGSVPPP

SEQ ID NO: 302 - protein, 20 aa, Homo sapiens
LDQYVKFDAVSGDYYPIIYF

SEQ ID NO: 303 - protein, 20 aa, Homo sapiens
NDYWNLQKDYYPINESLASL

SEQ ID NO: 304 - protein, 20 aa, Homo sapiens
PLRVSFCPLSLWRWQLYAAQ

SEQ ID NO: 305 - protein, 20 aa, Homo sapiens
STKSPWNFLGDELYEQSDEE

SEQ ID NO: 306 - protein, 20 aa, Homo sapiens
YEQSDEEQDSVKVALLETNP

SEQ ID NO: 307 - protein, 20 aa, Homo sapiens
LWRWQLYAAQSTKSPWNFLG

SEQ ID NO: 308 - protein, 20 aa, Homo sapiens
YPINESLASLPLRVSFCPLS

SEQ ID NO: 309 - protein, 20 aa, Homo sapiens
GDYYPIIYFNDYWNLQKDYY

SEQ ID NO: 310 - protein, 20 aa, Homo sapiens
WQVIKGVLFRIFIIWAISSW

SEQ ID NO: 311 - protein, 20 aa, Homo sapiens
RNLFPKDTLMNLHVYISEHE

SEQ ID NO: 312 - protein, 20 aa, Homo sapiens
FTDFNATSALFWEQHDLVYG

SEQ ID NO: 313 - protein, 27 aa, Homo sapiens
GGAPRVASRNLFPKDTLMNLHVYISEH

SEQ ID NO: 314 - protein, 35 aa, Homo sapiens
LDQYVKFDAVSGDYYPIIYFNDYWNLQKDYYPINE

SEQ ID NO: 315 - protein, 354 aa, Homo sapiens
MAAAQEADGARSAVVAAGGGSSGQVTSNGSIGRDPPAETQPQNPPAQPAPNAWQVIKGVLFRIFIIWAISSWFRRGPAPQDQAGPGGAPRVASRNLFPKDTLMNLHVYISEHEHFTDFNATSALFWEQHDLVYGDWTSGENSDGCYEHFAELDIPQSVQQNGSIYIHVYFTKSGFHPDPRQKALYRRLATVHMSRMINKYKRRRFQKTKNLLTGETEADPEMIKRAEDYGPVEVISHWHPNITINIVDDHTPWVKGSVPPPLDQYVKFDAVSGDYYPIIYFNDYWNLQKDYYPINESLASLPLRVSFCPLSLWRWQLYAAQSTKSPWNFLGDELYEQSDEEQDSVKVALLETNP

SEQ ID NO: 316 - protein, 20 aa, Homo sapiens
QLEIKYDFLYEKEHICCLEE

SEQ ID NO: 317 - protein, 19 aa, Homo sapiens
QLEIKYDFLYEKEHICCLE

SEQ ID NO: 318 - protein, 18 aa, Homo sapiens
QLEIKYDFLYEKEHICCL

SEQ ID NO: 319 - protein, 15 aa, Homo sapiens
HVYISEHEHFTDFNA

SEQ ID NO: 320 - protein, 15 aa, Homo sapiens
SEHEHFTDFNATSAL

SEQ ID NO: 321 - protein, 15 aa, Homo sapiens
VPPPLDQYVKFDAVS

SEQ ID NO: 322 - protein, 3 aa, Homo sapiens
PKD

SEQ ID NO: 323 - protein, 66 aa, Artificial Sequence
  AP1
AAAQEADGARSAVVAAGGGSSGQVTSNGSIGRDPPAETQPQNPPAQPAPNAGGGGSGGGGSGGGGS

SEQ ID NO: 324 - protein, 15 aa, Homo sapiens
LYEKEHICCLEEWTS

SEQ ID NO: 325 - protein, 15 aa, Homo sapiens
YEKEHICCLEEWTSP

SEQ ID NO: 326 - protein, 15 aa, Homo sapiens
EKEHICCLEEWTSPV

SEQ ID NO: 327 - protein, 15 aa, Homo sapiens
KEHICCLEEWTSPVH

SEQ ID NO: 328 - protein, 15 aa, Homo sapiens
EHICCLEEWTSPVHQ

SEQ ID NO: 329 - protein, 15 aa, Homo sapiens
HICCLEEWTSPVHQK

SEQ ID NO: 330 - protein, 15 aa, Homo sapiens
ISEHEHFTDFNATSA

SEQ ID NO: 331 - protein, 14 aa, Homo sapiens
EHEHFTDFNATSAL

SEQ ID NO: 332 - protein, 15 aa, Homo sapiens
HEHFTDFNATSALFW

SEQ ID NO: 333 - protein, 15 aa, Homo sapiens
EHFTDFNATSALFWE

SEQ ID NO: 334 - protein, 15 aa, Homo sapiens
HFTDFNATSALFWEQ

SEQ ID NO: 335 - protein, 6 aa, Homo sapiens
QQLEIK

SEQ ID NO: 336 - protein, 6 aa, Homo sapiens
DYYPII

SEQ ID NO: 337 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(9): X can be any naturally occuring amino acid
XXXXXXXXX

SEQ ID NO: 338 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(9): X can be any naturally occuring amino acid
XXXXXXXXX

SEQ ID NO: 339 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(9): X can be any naturally occuring amino acid
XXXXXXXXX

SEQ ID NO: 340 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(9): X can be any naturally occuring amino acid
XXXXXXXXX

SEQ ID NO: 341 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(9): X can be any basic amino acid
XXXXXXXXX

SEQ ID NO: 342 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  misc_feature (5)..(5): Xaa can be any naturally occurring amino acid
  MISC_FEATURE (6)..(9): X can be any naturally occuring amino acid
XXXXXXXXX

SEQ ID NO: 343 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be any K, R or H
  MISC_FEATURE (6)..(9): X can be any any naturally occuring amino acid
XXXXXXXXX

SEQ ID NO: 344 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(9): X can be any naturally occuring amino acid
XXXXXXXXX

SEQ ID NO: 345 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(9): X can be any naturally occuring amino acid
XXXXXXXXX

SEQ ID NO: 346 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 347 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 348 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(9): X can be any naturally occuring amino acid
XXXXXXXXX

SEQ ID NO: 349 - protein, 9 aa, Homo sapiens
  MISC_FEATURE (1)..(1): X can be F, W or Y
  MISC_FEATURE (2)..(2): X can be L, V, I, A or M
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 350 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 351 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 352 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 353 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 354 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 355 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 356 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(9): X can be any naturally occuring amino acid
XXXXXXXXX

SEQ ID NO: 357 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 358 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 359 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 360 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 361 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 362 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(9): X can be any naturally occuring amino acid
XXXXXXXXX

SEQ ID NO: 363 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(9): X can be any naturally occuring amino acid
XXXXXXXXX

SEQ ID NO: 364 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(9): X can be any naturally occuring amino acid
XXXXXXXXX

SEQ ID NO: 365 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(9): X can be any naturally occuring amino acid
XXXXXXXXX

SEQ ID NO: 366 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(9): X can be any naturally occuring amino acid
XXXXXXXXX

SEQ ID NO: 367 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(9): X can be any naturally occuring amino acid
XXXXXXXXX

SEQ ID NO: 368 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(9): X can be any naturally occuring amino acid
XXXXXXXXX

SEQ ID NO: 369 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 370 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 371 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 372 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 373 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 374 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 375 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  misc_feature (5)..(5): Xaa can be any naturally occurring amino acid
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 376 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be any K, R or H
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 377 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 378 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 379 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 380 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 381 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 382 - protein, 9 aa, Homo sapiens
  MISC_FEATURE (1)..(1): X can be F, W or Y
  MISC_FEATURE (2)..(2): X can be L, V, I, A or M
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 383 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(4): X can be any naturally occuring amino acid
  MISC_FEATURE (5)..(5): X can be K, R or H
  MISC_FEATURE (6)..(6): X can be any acidic amino acid
  MISC_FEATURE (7..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 384 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 385 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 386 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 387 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 388 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 389 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 390 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 391 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 392 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 393 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 394 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 395 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any naturally occuring amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 396 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any naturally occuring amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 397 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any naturally occuring amino acid
  MISC_FEATURE (5)..(5): X can be any basic amino acid
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 398 - protein, 9 aa, Homo sapiens
  MISC_FEATURE (1)..(1): X can be any aromatic amino acid
  MISC_FEATURE (2)..(2): X can be any aliphatic amino acid
  MISC_FEATURE (3)..(3): X can be any aromatic amino acid
  MISC_FEATURE (4)..(4): X can be any acidic amino acid
  MISC_FEATURE (5)..(5): X can be any basic amino acid
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 399 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any naturally occuring amino acid
  MISC_FEATURE (5)..(5): X can be K, R or H
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 400 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any naturally occuring amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 401 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any naturally occuring amino acid
  MISC_FEATURE (8)..(8): X can be any aliphatic amino acid
  MISC_FEATURE (9)..(9): X can be any naturally occuring amino acid
XXXXXXXXX

SEQ ID NO: 402 - protein, 9 aa, Homo sapiens
  MISC_FEATURE (1)..(1): X can be F, W or Y
  MISC_FEATURE (2)..(2): X can be L, V, I, A or M
  MISC_FEATURE (3)..(3): X can be any aromatic amino acid
  MISC_FEATURE (4)..(4): X can be any acidic amino acid
  MISC_FEATURE (5)..(5): X can be K, R or H
  MISC_FEATURE (6)..(6): X can be any acidic amino acid
  MISC_FEATURE (7)..(7): X can be any basic amino acid
  MISC_FEATURE (8)..(8): X can be any aliphatic amino acid
  MISC_FEATURE (9)..(9): X can be any naturally occuring amino acid
XXXXXXXXX

SEQ ID NO: 403 - protein, 9 aa, Homo sapiens
  MISC_FEATURE (1)..(1): X can be F, W or Y
  MISC_FEATURE (2)..(2): X can be L, V or I
  MISC_FEATURE (3)..(3): X can be F, W or Y
  MISC_FEATURE (4)..(4): X can be D or E
  MISC_FEATURE (5)..(5): X can be K, R or H
  MISC_FEATURE (6)..(6): X can be D or E
  MISC_FEATURE (7)..(7): X can be K, R or H
  MISC_FEATURE (8)..(8): X can be L, V or I
XXXXXXXXC

SEQ ID NO: 404 - protein, 10 aa, Homo sapiens
  MISC_FEATURE (1)..(1): X can be D or E
  MISC_FEATURE (2)..(2): X can be K, R or H
  MISC_FEATURE (3)..(3): X can be D or E
  MISC_FEATURE (4)..(4): X can be F, W or Y
  MISC_FEATURE (5)..(5): X can be D or E
  MISC_FEATURE (6)..(6): X can be D or E
  MISC_FEATURE (7)..(7): X can be L, V, I, A or M
  MISC_FEATURE (8)..(8): X can be A, S or T
  MISC_FEATURE (9)..(9): X can be L, V, I, A or M
  MISC_FEATURE (10)..(10): X can be R, K or H
XXXXXXXXXX

SEQ ID NO: 405 - protein, 10 aa, Homo sapiens
  MISC_FEATURE (1)..(1): X can be D or E
  MISC_FEATURE (2)..(2): X can be K, R or H
  MISC_FEATURE (3)..(3): X can be D or E
  MISC_FEATURE (4)..(4): X can be F, W or Y
  MISC_FEATURE (5)..(5): X can be D or E
  MISC_FEATURE (6)..(6): X can be D or E
  MISC_FEATURE (7)..(7): X can be L, V or I
  MISC_FEATURE (8)..(8): X can be A, S or T
  MISC_FEATURE (9)..(9): X can be L, V or I
  MISC_FEATURE (10)..(10): X can be R, K or H
XXXXXXXXXX